(19) United States
(12) Patent Application Publication
(10) Pub. No.: US 2024/0075182 A1
(43) Pub. Date: Mar. 7, 2024

(54) ENHANCED DERMAL PATCH FOR TREATING MENSTRUAL PAIN

(71) Applicants: Brent E. Cahill, Laguna Beach, CA (US); Mark Burleson, Irvine, CA (US); Ludwig Weiman, Vista, CA (US)

(72) Inventors: Brent E. Cahill, Laguna Beach, CA (US); Mark Burleson, Irvine, CA (US); Ludwig Weiman, Vista, CA (US)

(73) Assignee: REMY BIOSCIENCES, Santa Ana, CA (US)

(21) Appl. No.: 17/962,543

(22) Filed: Oct. 9, 2022

Related U.S. Application Data
(63) Continuation of application No. 17/706,869, filed on Mar. 29, 2022.
(60) Provisional application No. 63/167,498, filed on Mar. 29, 2021.

(51) Int. Cl. A61L 15/44; A61F 13/02

(52) U.S. Cl. CPC A61L 15/44; A61F 13/0206; A61F 13/0253

(57) ABSTRACT

The disclosure provides monolithic-style dermal patches and reservoir-style dermal patches, where the dermal patch is capable of delivering a mixture of anti-pain drugs to the skin. Provided also are methods for manufacturing said dermal patches.

### FIELD OF THE DISCLOSURE

The disclosure relates to dermal patches that comprise one or more drugs that can relieve menstrual pain, and that are capable of delivering to the skin said one or more drugs. Dysmenorrhea occurs as primary dysmenorrhea (PD), which appears not to be caused by any pathological pelvic diseases. Dysmenorrhea also occurs as secondary dysmenorrhea (SD), which is caused by a pathological pelvic disease.

### BACKGROUND OF THE DISCLOSURE

Dermal patches can take the form of a monolithic-style patch, reservoir-style patch, or hydrogel patch (see, US2017/0071870 of Weimann, which is incorporated herein in its entirety). Monolithic-style patches can take the form of a sandwich, where the face that is exposed to the atmosphere is a backing, where the opposite face is a release liner, and where the filling of the sandwich is a matrix that includes an adhesive and one or more pharmaceutical agents such as an anti-pain drug. Prior to applying the patch to the skin, the release liner is removed and discarded.

Regarding reservoir-style patches, the reservoir can contain one or more pharmaceutical agents such as an anti-pain drug. The reservoir can be defined by a backing and by a permeable membrane, which together assume a “ravioli” conformation. The permeable membrane is optionally coated with an adhesive that mediates binding of the adhesive to the skin. On one side of the adhesive is the permeable membrane, and on the other side is a release liner. Prior to applying the patch to the skin, the release liner is removed and discarded.

Hydrogel patches, like monolithic-style patches, can take the form of a sandwich, where the face that is exposed to the atmosphere is a backing, where the opposite face is a release liner, and where the filling of the sandwich is a hydrogel, a semisolid three-dimensional, crosslinked networks of synthetic and natural water-soluble polymers. This hydrogel can contain one or more water-soluble pharmaceutical agents such as an anti-pain drug. Prior to applying the patch to the skin, the release liner is removed and discarded.

Regarding the adhesive, in dermal patches it is common for this to be a pressure sensitive adhesive, which can flow and wet the skin surface in order to form a bond to the skin, and to be used as a carrier for the drug. Common adhesives in dermal patches include polyisobutylene (PIB), polyacrylate, and polydimethylsiloxane.

### SUMMARY OF THE DISCLOSURE

Regarding the following dermal patch embodiments, each of these embodiments may correspond to a monolithic-style dermal patch, to a reservoir-style dermal patch, to a hydrogel-style dermal patch, or to any combination of monolithic-style, reservoir-style and hydrogel-style dermal patches. This correspondence can be determined where there is an explicit use, in the claim, of the word, “monolithic,” “reservoir,” or “hydrogel.” Also, this correspondence can be determined by the recitation in the base claim (in the claim set), of the word “monolithic,” “reservoir,” or “hydrogel.”

The present disclosure provides a pharmaceutical formulation that, when applied to the skin, is capable of reducing menstrual pain, wherein the composition comprises at least one of capsaicin, eugenol, diclofenac, a cannabinoid, or a terpene or terpenoid present in Cannabis sativa wherein said pharmaceutical formulation further comprises a penetration enhancer.

Also provided is the above formulation wherein said penetration enhancer is one or more of either azone, oleic acid, dihydromyricetin, limonene, dimethylsulfoxide (DMSO), 1,2-propylene glycol, 2-(2-Ethoxyethoxy)ethanol (Transcutol®), isopentenyl pyrophosphate (IPP), isopropyl myristate, dimethyl isosorbide (DMI), d-Limonene, oleic acid, benzyl benzoate, caprylic monoglyceride, ethanol, 1-menthol, and Labrasol®. This list is not all-inclusive and those skilled in the art can recognize that other solvents, or co-solvents, can be combined to both dissolve active ingredients and enhance skin penetration. One such example is Medium Chain Triglycerides (MCT) oil.

Also provided is the above promulgation, wherein the cannabinoid comprises cannabidiol (CBD). Also provided is the above formulation, wherein the cannabinoid comprises cannabidiolic acid (CBDa). Moreover, the present disclosure embraces the above pharmaceutical formulation, wherein the cannabinoid consists of only one kind of cannabinoid. In another aspect, what is embraced is the above formulation, wherein the at cannabinoid comprises one or more of cannabidiol (CBD), cannabichromene (CBC), cannabigerol (CBG), delta-9-tetrahydrocannabinol (delta-9-THC), cannabidiolic acid (CBDa), and cannabinol (CBN). In exclusionary embodiments, what is provided is the above formulation that excludes one or more of opioids, muscle relaxants, corticosteroids, antianxiety drugs, antidepressants, and anticonvulsant drugs.

Also provided is the above promulgation, wherein the terpene of Cannabis sativa comprises β-caryophyllene (BCAP). Also provided is the above formulation, wherein the terpene comprises limonene. Also provided is the above formulation, wherein the terpene comprises linalool. Also provided is the above formulation, wherein the terpene comprises α-pinene. Also provided is the above formulation, wherein the terpene comprises β-pinene. Also provided is the above formulation, wherein the terpene comprises β-myrcene. Moreover, the present disclosure embraces the above pharmaceutical formulation, wherein the terpene consists of only one kind of terpene. In another aspect, what is embraced is the above formulation, wherein the terpene comprises one or more of β-caryophyllene (BCAP), limonene, linalool, α-pinene, β-myrcene, α-bisabolol, eucalyptol, trans-nerolidol, humulene, delta-β-carene, camphene, borneol, terpineol, valencene, and geraniol. In exclusionary embodiments, what is provided is the above formulation that excludes one or more of opioids, muscle relaxants, corticosteroids, antianxiety drugs, antidepressants, and anticonvulsant drugs.

Additionally, what is provided is the above pharmaceutical formulation, that comprises one of the following lists of chemical ingredients: (i) eugenol and capsaicin, (ii) eugenol, capsaicin, and cannabidiol (CBD), eugenol, capsaicin, and β-caryophyllene (BCAP), (iii) eugenol, capsaicin, and diclofenac, (iv) eugenol, capsaicin, and cannabidiolic acid (CBDA), (v) eugenol, capsaicin, cannabidiol (CBD), and cannabidiolic acid (CBDA) (vi) eugenol, capsaicin, diclofenac, and β-caryophyllene (BCAP), (vii) eugenol, capsaicin, cannabidiol (CBD), cannabidiolic acid (CBDA), and diclofenac, (viii) eugenol, capsaicin, cannabidiol (CBD), cannabidiolic acid (CBDA), diclofenac, and β-caryophyllene (BCAP).

In yet another aspect, what is provided is the above pharmaceutical formulation that includes one or more of a polyisobutylene (PIB) adhesive, ascorbyl palmitate, a tackifier in the form of a resin, and one or more permeation enhancers.

In yet another aspect, what is provided is the above pharmaceutical formulation that includes one or more of a polydimethylsiloxane adhesive, ascorbyl palmitate, a tackifier in the form of a resin, and one or more permeation enhancers.

In yet another aspect, what is provided is the above pharmaceutical formulation that includes one or more of a polyacrylate adhesive, ascorbyl palmitate, a tackifier in the form of a resin, and one or more permeation enhancers.

In dermal patch embodiments, what is provided is a dermal patch comprising a pharmaceutical formulation capable of reducing menstrual pain when applied to the skin, wherein the composition comprises diclofenac, capsaicin, and cannabidiol (CBD).

In dermal patch embodiments, what is provided is a dermal patch comprising a pharmaceutical formulation capable of reducing menstrual pain when applied to the skin, wherein the composition comprises diclofenac, capsaicin, and β-caryophyllene (BCAP).

Additional dermal patch embodiments include the above dermal patch, wherein the menstrual pain results from primary dysmenorrhea or from secondary dysmenorrhea. Provided is the above dermal patch that is a reservoir-style dermal patch, as well as the above dermal patch that is a monolithic-style dermal patch, as well as the above dermal patch that is a hydrogel-style patch.

Also encompassed, is the above dermal patch that comprises a flexible tan polyethylene, polyolefin, or polyurethane foam backing layer on a clear fluorosilicone or fluoropolymer-coated polyester release liner, wherein the foam backing is the part of the patch that occludes the active ingredients from the external environment, wherein the foam backing is the part of the patch that is placed on the skin, and wherein the release liner is the clear plastic sheet surrounding the patch that covers said foam backing, and wherein said release liner is removable by peeling it from the patch.

In yet another aspect, what is provided is the above dermal patch, wherein the diclofenac is either diclofenac sodium or diclofenac epolamine, and the above dermal patch, wherein the diclofenac is either diclofenac sodium or diclofenac epolamine, and wherein the concentration of diclofenac sodium in the formulation is about 1.0 percent and wherein the concentration of diclofenac epolamine is about 1.3 percent, and the above dermal patch, wherein the concentration of capsaicin in the formulation is about 0.025%, about 0.05%, about 0.1%, or about 0.15%, as well as the above dermal patch, wherein the total amount of eugenol is about 10 milligrams, about 15 mg, or about 20 mg.

In other embodiments, what is provided is the above dermal patch, wherein the concentration of capsaicin in the formulation is about 0.025%, about 0.05%, about 0.1%, or about 0.15%, as well as the above dermal patch, wherein the total amount of eugenol is about 10 milligrams, about 15 mg, or about 20 mg, as well as the above dermal patch wherein the total amount of Cannabis sativa terpenes are about 20 mg, about 25 mg, about 30 mg, or about 35 mg.

What is further provided in other embodiments is the above dermal patch wherein the terpene is comprised of one or more of the following: β-caryophyllene (BCAP), limonene, linalool, α-pinene, β-myrcene, or geraniol.

Furthermore, in other embodiments, what is provided the above dermal patch, wherein the dermal patch is capable of reducing anxiety or depression that results from menstrual pain, as well as the above dermal patch, wherein the pharmaceutical formulation contains a total of about 35 milligrams of one or more cannabinoids, and the above dermal patch, wherein the pharmaceutical formulation contains a total amount of about 10 milligrams of eugenol, and the above dermal patch, wherein the pharmaceutical formulation contains a total amount of about 35 milligrams of cannabinoids.

In exclusionary embodiments, what is provided is the above dermal patch, wherein said pharmaceutical formulation excludes one or more or all of opioids, muscle relaxants, corticosteroids, antianxiety drugs, antidepressants, and anticonvulsant drugs.

In embodiments that include adhesives, antioxidants, tackifiers, and enhancers, what is provided is the above dermal patch, that includes one or more of a polyisobutylene (PIB) adhesive, ascorbyl palmitate, a tackifier in the form of a resin, and one or more permeation enhancers.

In embodiments that include adhesives, antioxidants, tackifiers, and enhancers, what is provided is the above dermal patch, that includes one or more of a polydimethylsiloxane adhesive, ascorbyl palmitate, a tackifier in the form of a resin, and one or more permeation enhancers.

In embodiments that include adhesives, antioxidants, tackifiers, and enhancers, what is provided is the above dermal patch, that includes one or more of a polyacrylate adhesive, ascorbyl palmitate, a tackifier in the form of a resin, and one or more permeation enhancers.

In all embodiments, what is provided is the above dermal patch, that includes a blend of one or more of polyisobutylene (PIB), polydimethylsiloxane, or polyacrylate adhesives.

In pharmaceutical embodiments, what is provided is a pharmaceutical formulation that, when applied to the skin, is capable of reducing pain associated with primary dysmenorrhea, wherein the composition comprises one of the following lists of chemical ingredients: (i) eugenol and capsaicin, (ii) eugenol, capsaicin, and cannabidiol (CBD), eugenol, capsaicin, and β-caryophyllene (BCAP), (iii) eugenol, capsaicin, and diclofenac, (iv) eugenol, capsaicin, and cannabidiolic acid (CBDA), (v) eugenol, capsaicin, cannabidiol (CBD), and cannabidiolic acid (CBDA) (vi) eugenol, capsaicin, diclofenac, and β-caryophyllene (BCAP), (vii) eugenol, capsaicin, cannabidiol (CBD), cannabidiolic acid (CBDA), and diclofenac, (viii) eugenol, capsaicin, cannabidiol (CBD), cannabidiolic acid (CBDA), diclofenac, and β-caryophyllene (BCAP).

Also embraced is the above pharmaceutical formulation, wherein said “capable of reducing pain associated with primary dysmenorrhea” is experienced by a patient who is treated with a dermal patch that delivers said pharmaceutical formulation, and wherein said “capable of reducing pain associated with primary dysmenorrhea” is as compared with said patient prior to attaching said dermal patch to said patient, and wherein the degree of menstrual pain prior to and after attaching said dermal patch to the skin of said patient is measured by a scoring tool. Further embraced is the above pharmaceutical formulation, that excludes one or more or all of opioids, muscle relaxants, corticosteroids, antianxiety drugs, antidepressants, and anticonvulsant drugs.

Several additional dermal patch embodiments are provided, as disclosed below, and these encompass monolithic-style dermal patches and reservoir-style dermal patches. What is provided is a dermal patch comprising one of the pharmaceutical formulations, as set forth in the following lists of chemical ingredients: (i) eugenol and capsaicin, (ii) eugenol, capsaicin, and cannabidiol (CBD), eugenol, capsaicin, and β-caryophyllene (BCAP), (iii) eugenol, capsaicin, and diclofenac, (iv) eugenol, capsaicin, and cannabidiolic acid (CBDA), (v) eugenol, capsaicin, cannabidiol (CBD), and cannabidiolic acid (CBDA) (vi) eugenol, capsaicin, diclofenac, and β-caryophyllene (BCAP), (vii) eugenol, capsaicin, cannabidiol (CBD), cannabidiolic acid (CBDA), and diclofenac, (viii) eugenol, capsaicin, cannabidiol (CBD), cannabidiolic acid (CBDA), diclofenac, and β-caryophyllene (BCAP), wherein said “capable of reducing pain associated with primary dysmenorrhea” is with regard to the pain associated with primary dysmenorrhea experienced by the same patient but prior to attaching said dermal patch to said patient, and wherein the degree of menstrual pain prior to and after attaching said dermal patch to the skin of said patient are both measured using the same scoring tool.

In pain scale embodiments, what is provided is the above dermal patch, wherein said menstrual pain before and after treatment with said dermal patch is measured using the Visual Analogue Scale (VAS), Cox Menstrual Symptom Scale (CMSS)-interpreted menstruation symptoms score, Quality of Life Scale (QOLS)-interpreted quality of life score, the Flanagan Quality of Life (QOL) score, Verbal Multidimensional Scoring system (VMS) score, McGill Pain Questionnaire (MPQ) score, Profile of Mood States (POMS) Questionnaire, the Short Form-36 (SF-36) score, Profile of Mood States (POMS) Questionnaire score, or the Numeric Rating Scale (NRS) score.

In flexible tan foam backing embodiments, what is provided is the above dermal patch, that comprises a flexible tan polyethylene, polyolefin, or polyurethane foam backing layer on a clear polyester release liner, wherein the foam backing is the part of the patch that occludes the active ingredients from the external environment, wherein the foam backing is the part of the patch that is placed on the skin, and wherein the release liner is the clear plastic sheet surrounding the patch that covers said foam backing, and wherein said release liner is removable by peeling it from the patch.

In exclusionary embodiments of dermal patch embodiments, what is provided is the above dermal patch, wherein one or more or all of opioids, muscle relaxants, corticosteroids, antianxiety drugs, antidepressants, and anticonvulsant drugs, are excluded from said pharmaceutical formulation. Also provided is the above dermal patch, that includes one or more of a polyisobutylene (PIB) adhesive, a polydimethylsiloxane adhesive, a polyacrylate adhesive, ascorbyl palmitate, a tackifier in the form of a resin, and one or more permeation enhancers.

In permeation enhancer embodiments, what is provided is the above dermal patch of, wherein said one or more permeation enhancers comprises azone, oleic acid, isopentenyl pyrophosphate (IPP), dimethylsulfoxide (DMSO), propylene glycol (1,2 PG), or isopropyl myristate (IPM), dihydromyricetin, limonene, or 2-(2-Ethoxyethoxy)ethanol (Transcutol®).

Method for Manufacturing Monolithic-Style Patch, with Reference to a Dermal Patch that can Comprise Only One List of Drugs (Eugenol, Capsaicin, β-Caryophyllene (BCAP)), or a Dermal Patch Comprised of Only One List of Drugs that is Selected from a Group of Many Different Lists of Drugs

The first list, in all its entirety, takes the following form: A dermal patch comprising a pharmaceutical formulation capable of reducing menstrual pain when applied to the skin, wherein the composition comprises eugenol, capsaicin, and β-caryophyllene (BCAP).

The present disclosure encompasses methods for manufacturing one of the above disclosed dermal patches (dermal patch with first collection of drug lists, and dermal patch with second collection of drug lists), where the patch is a monolithic patch.

What is provided is a method for manufacturing a monolithic-style dermal patch, the method comprising:
Step (i). Dissolving an antioxidant in the form of ascorbyl palmitate in a solvent or solvent system, which may or may not include a penetration enhancer,
Step (ii). Mixing active pharmaceutical ingredients, wherein one or more of the active pharmaceutical ingredients are capable of reducing menstrual pain after topical and/or transdermal administration, wherein said active pharmaceutical ingredients are dissolved in said solvent or solvent system,
Step (iii). Filtering said mixture, then mixing with solvent for the adhesive and a cosolvent, if necessary,
Step (iv). Mixing the active pharmaceutical ingredient-solvent mixture with a pressure sensitive adhesive provided in solvent,
Step (v). Degassing said mixture in a vacuum in order to remove residual air bubbles,
Step (vi). Using the solvent casting method, cast the mixture via knife-over-roll onto a polyester release liner,
Step (vii). Evaporating the solvents from the mixture in an oven, wherein said oven is kept at about 85-95 degrees centigrade for at least one minute, resulting in a dried coat, wherein said dried coat has a weight that is about 50 grams per square meter (gsm),
Step (viii). Laminating the release liner to an occlusive backing, wherein the occlusive backing is made of polyethylene, polyolefin, or polyurethane foam,
Step (ix). Cutting the assembled release liner and occlusive backing into rectangles that have rounded corners, wherein the occlusive backing is cut into smaller rectangles than the release liner, resulting in a “kiss-cut” and wherein the “kiss-cut” occlusive backing rectangles have an area of about 30, about 40, or about 50 square centimeters.

Also included is the above method, wherein said active pharmaceutical ingredients comprises one or more of eugenol, capsaicin, cannabidiol (CBD), cannabidiolic acid (CBDA), diclofenac, and β-caryophyllene (BCAP). In another aspect, what is included is the above method, wherein the mixing step includes adding one or more permeation enhancers. Also embraced is the above method, wherein said mixing involves using an ultra-high shear dispersion blade.

Furthermore, what is provided is the above method, wherein the solvent for dissolving said active pharmaceutical ingredients comprises ethanol or methanol. Provided is the above method, wherein said adhesive includes a tackifier. In yet another aspect, provided is the above method, wherein the mixing step includes adding one or more preservatives, wherein said one or more preservatives are optionally dissolved in ethanol or methanol. Also provided is the above method, wherein the mixing step includes adding a tackifier adhesive dissolved in heptane or ethyl acetate. Also provided is the above method wherein ethyl acetate is used as a cosolvent.

Method for Manufacturing Reservoir-Style Patch, with Reference to a Dermal Patch that can Comprises Only One List of Drugs (Eugenol, Capsaicin, and β-Caryophyllene (BCAP), or a Dermal Patch Comprising Only One List of Drugs that is Selected from a Group of Many Different Lists of Drugs.

The first list, in all its entirety, takes the following form: A dermal patch comprising a pharmaceutical formulation capable of reducing menstrual pain when applied to the skin, wherein the composition comprises eugenol, capsaicin, and β-caryophyllene (BCAP). A reservoir-style transdermal delivery device comprises a backing and a hydrophilic, porous membrane. The backing and hydrophilic, porous membrane are attached to one another so as to define a closed volume which acts as a reservoir. A preparation comprising one or more of eugenol, capsaicin, cannabidiol (CBD), cannabidiolic acid (CBDA), diclofenac, and β-caryophyllene (BCAP), as well as a liquid carrier, and potentially a gelling agent in the reservoir. The first side of the hydrophilic, porous membrane is in contact with the preparation. A second side of the hydrophilic, porous membrane faces away from backing and is coated with a skin adhesive. The skin adhesive is preferably formulated to adhere the device to the user's skin for a period of 4 or 6 or 8 or 12 or 24 hours while avoiding appreciable skin irritation to the user's skin. Preferred skin adhesives include amine-compatible silicone pressure sensitive adhesives, polyacrylate pressure sensitive adhesives, and polyisobutylene adhesives. In certain embodiments, an amine-compatible silicone skin adhesive is provided which comprises a trimethylsiloxy end-capped reaction product of a silanol end-blocked polydimethylsiloxane and a silicate resin. The skin adhesive is preferably provided in an organic solvent solution comprising from about 50 percent to about 70 percent by weight of solid adhesive in an organic solvent like heptane or ethyl acetate and having a viscosity at 20° C. of from about 400 mPa-s to about 1300 mPa-s, preferably from about 450 mPa-s to about 1250 mPa-s, and more preferably from about 500 mPa-s to about 1200 mPa-s.

A first surface of a release liner is releasably adhered to skin adhesive, and a second surface of release liner faces away from skin adhesive. Suitable release liners include occlusive polymeric films, such as polyester or polypropylene, coated with a release coating that is releasably adherable to silicone, polyisobutylene, and silicone adhesives. Suitable release coatings on the first surface of the release liner include fluoropolymers and fluorosilicone polymers. Commercially-available coated release liners that are suitable for use as release liner include Scotchpak 1022, 9741, 9744, 9748, and 9755 supplied by 3M of Minneapolis, MN., and FRA 310, 314, 315, and 316 supplied by Fox River Co. To use the reservoir transdermal device, the release liner is peeled away from the skin adhesive, thereby exposing skin adhesive, and the device is applied so that the skin adhesive contacts the user's skin.

Suitable examples of adhesives include BIO-PSA 7-4301 and 7-4302 skin adhesives supplied by Dow Corning. BIO-PSA 7-4301 is a high tack, amine-compatible silicone adhesive in heptane available with a solids content of 60 percent to 70 percent and corresponding viscosities at 20 degrees C. of 450 mPa-s and 1600 mPa-s. BIO-PSA 7-4302 is a high tack, amine-compatible silicone adhesive in ethyl acetate with a solids content of 60 percent by weight and a viscosity of 1200 mPa-s at 20 degrees C. Also included are the polyacrylate adhesives DURO-TAK 87-2516, 87-2074, and 87-2194, among others provided by Henkel. Also included are polyisobutylene adhesives of various molecular weights provided by BASF. Also included are combinations of any grades and molecular weights of BASF polyisobutylene. Also included are commercially available polyisobutylene adhesives provided in heptane, such as 87-6908 from Henkel. The skin adhesive is coated to a thickness per unit area on the membrane that, when dry, is preferably from about 10 to about 80 g/square meters, more preferably from about 40-70 g/square meter, and still more preferably from about 45-60 g/square meter.

The hydrophilic, porous membrane preferably has a mean flow pore size of no more than about 1 micron, preferably not more than about 0.8 microns, still more preferably no more than about 0.4 microns, and even more preferably no more than about 0.2 microns. At the same time, the porous membrane preferably has a mean flow pore size of no less than about 0.02 microns, more preferably no less than about 0.04 microns, still more preferably no less than about 0.06 microns, and even more preferably no less than about 0.08 microns. The mean flow pore size may be determined in accordance with the method set forth at page 17, line 22 to page 18, line 4 of published PCT Application WO2010072233, the entirety of which is hereby incorporated by reference.

In the same or other examples, the hydrophilic porous membrane preferably has a porosity of at least about 60 percent, more preferably at least about 65 percent, and still more preferably at least about 70 percent. At the same time, hydrophilic porous membrane preferably has a porosity of no more than about 90 percent, more preferably no more than about 85 percent, and still more preferably no more than about 80 percent. Porosity values may be calculated as described at page 7, lines 24 to 27 of WO2010072233.

In the same or other examples, hydrophilic porous membrane preferably has a thickness of no more than about 50 microns, preferably no more than about 40 microns, and even more preferably no more than about 35 microns. At the same time, the hydrophilic porous membrane preferably has a thickness of no less than about 10 microns, more preferably no less than about 20 microns, and still more preferably no less than about 25 microns. Membrane thicknesses may be determined as described at page 18, lines 19-21 of WO2010072233.

In the same or other examples, the hydrophilic porous membrane preferably has an air permeability as determined by the Gurley Test Method (according to ISO 5636-5) that is preferably at least about 10 sec/50 ml, more preferably at least about 20 sec/50 ml, and still more preferably at least about 25 sec/50 ml. At the same time, the hydrophilic porous membrane preferably has an air permeability of no more than about 50 sec/50 ml, more preferably no more than about 40 sec/50 ml, and still more preferably no more than about 35 sec/50 ml.

In the same or other examples, the hydrophilic porous membrane preferably has a tensile strength in the machine direction as determined by ASTM D882-12 that is preferably at least about 10 MPa, more preferably at least about 15 MPa, and still more preferably at least about 20 MPa. In the same or other examples, the hydrophilic porous membrane preferably has a percent elongation in the machine direction as determined by ASTM D882-12 that is preferably at least about 10 percent, more preferably at least about 15 percent, and still more preferably at least about 20 percent.

The hydrophilic porous membrane preferably comprises at least one polymeric material. In one example, hydrophilic porous membrane comprises a polyolefin polymer and a hydrophilic component that comprises a hydrophilic polymer and optionally, a surfactant. As used herein, the term “hydrophilic” when used to describe a porous membrane refers to a membrane that at 20° C. provides a water flux for demineralized water through the membrane of at least 0.5 liters/(m² bar).

The content of the polyolefin polymer is preferably less than or equal to 98 percent by weight based on the total dry weight of the membrane, and the content of the hydrophilic component(s) is preferably at least 2 weight percent based on the total dry weight of the membrane. In certain preferred examples, the membrane is formed by combining the polyolefin polymer with the hydrophilic components(s) and optional additives with a solvent to form a blend in the form of a gel, a solution, or a homogeneous mixture, followed by extruding the blend. Suitable polyolefins (such as polyethylene), hydrophilic components, and additives are described in WO2010072233.

One example of a commercially available hydrophilic, porous membrane that is suitable for use as hydrophilic, porous membrane is supplied by Lydall Performance Materials B.V. under the name Evopor™ 5E02A. Evopor™ 5E02A is a porous hydrophilic membrane comprising a polyethylene support and a poly (ethyl vinyl) alcohol hydrophilic component. Hydrophobic Lydall® Solupor™ 7P03A porous membrane, or other similar types, can be employed.

As mentioned previously, the preparation comprises one or more of eugenol, capsaicin, cannabidiol (CBD), cannabidiolic acid (CBDA), diclofenac, and β-caryophyllene (BCAP) and a liquid carrier. In certain examples, the polar organic liquid comprises a molecule having one or more carboxylic acid groups. In the same or other examples, the polar organic liquid comprises a molecule having one or more hydroxyl groups. Suitable polar organic liquids comprising one or more hydroxyl groups include those comprising between 2 and 30 carbon atoms per molecule, including without limitation, ethanol. Suitable polar organic liquids comprising one or more carboxylic acid groups include fatty acids, including without limitation oleic acid. Liquid carriers comprising ethanol and/or oleic acid are preferred, and liquid carriers comprising oleic acid are especially preferred. Suitable liquid carriers also include mixtures of polar organic liquids and water. Examples of such mixtures include mixtures of ethanol and water. In ethanol/water mixtures, the maximum concentration of water is preferably about ten (10) percent by weight of the total amount of ethanol and water.

The preparation also may comprise a gelling agent which makes the preparation thixotropic. Suitable gelling agents include: sodium carboxymethyl cellulose, hydroxypropyl cellulose, hydroxyethyl cellulose, polyacrylic acid, methyl cellulose, xanthan gum, etc. In certain examples, cellulose gelling agents such as hydroxyethyl cellulose are preferred. The gelling agent increases the viscosity of and provides structural integrity to preparation, which improves the ease of placing and retaining preparation in the reservoir before the reservoir is closed by heat sealing the hydrophilic, porous membrane to the occlusive backing. The gelling agent is preferably pharmacologically inactive.

The gelling agents can also be classified as hydrogels, which are composed of either natural or synthetic gels that have at least one water-soluble polymer that can be compatible with ethanol or other solvents, penetration enhancers, surfactants, or gelling agents. These hydrogels can also contain a penetration enhancer such as, but not limited to, azone, oleic acid, dihydromyricetin, limonene, dimethylsulfoxide (DMSO), 1,2-propylene glycol, 2-(2-Ethoxyethoxy)ethanol (Transcutol®), isopentenyl pyrophosphate (IPP), isopropyl myristate, benzyl alcohol, isopropyl palmitate, polypropylene glycol, diethyl sebacate, etc.

Synthetic hydrogels include, but are not limited to, poly(acrylic acids), poly(2-hydroxyethyl methacrylate), poly(vinyl alcohol), poly(ethylene glycol), poly(ethylene oxide), poly(n-isopropylacrylamide), silicone, poly(vinyl pyrrolidone), and polyurethanes. The inventor considers polyurethane and similar polymers as a class that can undergo reversible thermal gelation at or near physiological temperatures, as well as being block co- or tri-polymers, where the chemical nature of the blocks can assist the structural rigidity and the solubility of the active agents. Other polymers in this class include poly(propylene oxide), poly(lactide-co-glycolic acid), poly(n-isopropylacrylamide), poly(propylene fumarate, poly(caprolactone), and poly(organophosphazene).

Natural hydrogels include, but are not limited to, alginates, hyaluronic acids, collagens, fibrins, and agarose.

This invention can utilize blends of synthetic and natural hydrogels.

The active ingredients which are comprised of one or more of eugenol, capsaicin, cannabidiol (CBD), cannabidiolic acid (CBDA), diclofenac, and β-caryophyllene (BCAP) are present in a therapeutically effective amount in preparation. A “therapeutically effective amount” is an amount of eugenol, capsaicin, and β-caryophyllene (BCAP) sufficient to achieve a desired therapeutic effect over a desired time period.

In certain examples, the preparation preferably comprises from about one (1) percent to about fifty (50) percent by weight of combined eugenol, capsaicin, and β-caryophyllene (BCAP), more preferably from about five (5) to about 30 percent by weight of combined eugenol, capsaicin, and β-caryophyllene (BCAP), and more preferably from about 10 to about 20 percent by weight of combined eugenol, capsaicin, and β-caryophyllene (BCAP).

In the same or other examples, the preparation comprises a liquid carrier in amounts ranging from about 50 percent to about 99 percent by weight of the preparation. The preparation also comprises from about one (1) percent to about ten (10) percent, and preferably about three (3) percent of a gelling agent (preferably hydroxypropyl cellulose) by weight of the preparation. The preparation also includes from zero to about ten (10) percent by weight of at least one penetration enhancer. In certain examples, the liquid carrier comprises at least one polar liquid of the type described previously. Suitable penetration enhancers include, but are not limited to azone, oleic acid, dihydromyricetin, limonene, dimethylsulfoxide (DMSO), 1,2-propylene glycol, 2-(2-Ethoxyethoxy)ethanol (Transcutol®), isopentenyl pyrophosphate (IPP), isopropyl myristate, benzyl alcohol, isopropyl palmitate, polypropylene glycol, diethyl sebacate, etc.

Suitable backing materials for backing include occlusive polymeric films such as polyethylene, polypropylene, polyolefin, polyethylene terephthalate (PET) and combinations thereof. Although the device may include an overlay patch, in preferred examples, one is not provided. In general, an overlay patch is not necessary if the hydrophilic, porous membrane is already coated with skin adhesive. If the membrane is not coated with an adhesive (e.g., in order to maximize the flux of eugenol, capsaicin, and β-caryophyllene (BCAP) into the skin), an overlay patch could be placed over the reservoir in order to ensure intimate contact of the hydrophilic, porous membrane with skin. In certain examples, the skin contact area (“active transdermal flux area”) of the membrane of a device is preferably at least about 10 cm², more preferably at least about 20 cm², and still more preferably at least about 30 cm². At the same time, the skin contact area of the device is preferably no more than about 80 cm², preferably no more than about 70 cm², and still more preferably no more than about 60 cm². At a given flux rate, the skin contact area may be selected to achieve the desired daily dose of desired active (or the dose over whatever time period is of therapeutic interest).

In embodiments, patch measures about 20 cm², about 22 cm², about 24, about 25, about 30, about 40, about 50, about 60 cm², and the like. These measurements refer to the area of the patch, and do not imply the shape of the patch. In embodiments, patch is square shaped, square shaped with rounded corners, oval shaped, rectangle shaped, rectangle shaped with rounded corners, circular, and so on.

What is provided is a method for manufacturing the above disclosed dermal patch (dermal patch comprising a pharmaceutical formulation capable of reducing menstrual pain when applied to the skin, wherein the composition comprises eugenol, capsaicin, and β-caryophyllene (BCAP)), wherein the dermal patch is a reservoir-style patch, wherein said reservoir contains a formulation, wherein said formulation includes one or more anti-pain drugs, the method comprising the steps of:
Step (i). Mixing by high-dispersion blade the drug matrix by combining active pharmaceutical ingredients, wherein one or more of the active pharmaceutical ingredients are capable of reducing menstrual pain after topical and/or transdermal administration, wherein said active pharmaceutical ingredients are dissolved in a solution that may or may not contain permeation enhancers, viscosity modifiers, antioxidants, and skin softening agents,
Step (ii). Making the reservoir by feeding two strips or webs (first strip and second strip) into a machine with rollers, wherein the rollers move the strips at the same speed, wherein a first face of the first strip is caused to contact a first face of the second strip, in preparation for heating the edges of the two strips thus sealing them together,
Step (iii). The step of using heaters resembling wheels or rollers to clamp down on the edges of the two strips, thereby creating a sandwich taking the form of a long closed tube,
Step (iv). The step of using transverse clamps to create separate pouches in the long closed tube, wherein the transverse clamps are heated and clamp down, thereby creating an unfilled pouch,
Step (v). The step of filling the unfilled pouch using a long tube that reaches down into the long closed tube, wherein said long tube fills each pouch as it is created, wherein said machine has a deposit station, and wherein said step of filling is performed at the deposit station,
Step (vi). The step of using said heated transverse clamps to clamp down on a previously-filled pouch, thereby creating a top seal on said previously-filled pouch, thereby creating a filled reservoir,
Step (vii). The step of providing a backing, a permeable layer, and a filled reservoir, wherein the backing has edges and the permeable layer has edges,
Step (viii). The step of contacting the filled reservoir to the backing and to the permeable layer, wherein said contacting further comprises attaching by way of an adhesive seal or by way of a heat seal, wherein permeable layer resides on the proximal side (side closer to the skin) of the reservoir, and wherein the backing resides on the distal side (side farther from the skin) of the reservoir,
Step (ix). The step of attaching the edges of the backing to the edges of the permeable layer to each other to prevent leaking of said formulation out of the dermal patch,
Step (x). The step of cutting around the created reservoir to create a single “ravioli” style reservoir dermal patch.

In another aspect, what is provided is the above method, wherein said one more anti-pain drugs include eugenol, capsaicin, and cannabidiol (CBD). What is also embraced is the above method, wherein said formulation comprises a penetration enhancer, and the above method, wherein said formulation comprises a preservative, and the above method, wherein said formulation comprises an antioxidant such as ascorbyl palmitate, butylated hydroxytoluene (BHT), butylated hydroxyanisole (BHA), tocopherol, ascorbyl palmitate, propyl gallate, ascorbic acid, or citric acid, and the above method wherein said formulation comprises a gelling agent, emulsifier, surfactant, or viscosity modifier. In yet another embodiment, what is provided is the above method, wherein the permeable layer comprises permeable polypropylene film, permeable polyethylene film, or permeable polyurethane film.

Method for Manufacturing Reservoir-Style Patch, with Reference to a Dermal Patch that Comprises a Second List of Drug Combinations.

What is provided is a method for manufacturing the above-disclosed dermal patch (dermal patch comprising one of the pharmaceutical formulations, as set forth in the following lists of chemical ingredients:

One or more from the list of eugenol, capsaicin, cannabidiol (CBD), cannabidiolic acid (CBDA), diclofenac, and β-caryophyllene (BCAP).
wherein the dermal patch is a reservoir-style patch, wherein said reservoir contains a formulation, wherein said formulation includes one or more anti-pain drugs, the method comprising the steps of:
Step (i). Mixing by high-dispersion blade the drug matrix by combining active pharmaceutical ingredients, wherein one or more of the active pharmaceutical ingredients are capable of reducing menstrual pain after topical and/or transdermal administration, wherein said active pharmaceutical ingredients are dissolved in a solution that may or may not contain permeation enhancers, viscosity modifiers, antioxidants, and skin softening agents,
Step (ii). Making the reservoir by feeding two strips (first strip and second strip) into a machine with rollers, wherein the rollers move the strips at the same speed, wherein a first face of the first strip is caused to contact a first face of the second strip, in preparation for heating the edges of the two strips thus sealing them together,
Step (iii). The step of using heaters resembling wheels or rollers to clamp down on the edges of the two strips, thereby creating a sandwich taking the form of a long closed tube,
Step (iv). The step of using transverse clamps to create separate pouches in the long closed tube, wherein the transverse clamps are heated and clamp down, thereby creating an unfilled pouch,
Step (v). The step of filling the unfilled pouch using a long tube that reaches down into the long closed tube, wherein said long tube fills each pouch as it is created, wherein said machine has a deposit station, and wherein said step of filling is performed at the deposit station,
Step (vi). The step of using said heated transverse clamps to clamp down on a previously-filled pouch, thereby creating a top seal on said previously-filled pouch, thereby creating a filled reservoir,
Step (vii). The step of providing a backing, a permeable layer, and a filled reservoir, wherein the backing has edges and the permeable layer has edges,
Step (viii). The step of contacting the filled reservoir to the backing and to the permeable layer, wherein said contacting further comprises attaching by way of an adhesive seal or by way of a heat seal, wherein permeable layer resides on the proximal side (side closer to the skin) of the reservoir, and wherein the backing resides on the distal side (side farther from the skin) of the reservoir,
Step (ix). The step of attaching the edges of the backing to the edges of the permeable layer to each other to prevent leaking of said formulation out of the dermal patch,
Step (x). The step of cutting around the created reservoir to create a single “ravioli” style reservoir dermal patch.

In another aspect, what is provided is the above method, wherein said one more anti-pain drugs include eugenol, capsaicin, and cannabidiol (CBD). What is also embraced is the above method, wherein said formulation comprises a penetration enhancer, and the above method, wherein said formulation comprises a preservative, and the above method, wherein said formulation comprises an antioxidant such as ascorbyl palmitate, and the above method wherein said formulation comprises a viscosity modifier. In yet another embodiment, what is provided is the above method, wherein the permeable layer comprises permeable polypropylene film, permeable polyethylene film, or permeable polyurethane film.

### DRAWINGS

FIG. 1 is a side cross-sectional view of a reservoir-style transdermal device for delivering CBD to a wearer's skin. Reservoir-style dermal patch 20, backing 22, hydrophilic, porous membrane 24. The backing 22 and hydrophilic, porous membrane 24 are attached to one another so as to define a closed volume which acts as a reservoir 26. Drug formulation (or composition) 27. First side 34 of the hydrophilic, porous membrane 24 is in contact with the preparation 27. A second side 36 of the hydrophilic, porous membrane 24 faces away from backing 22 and is coated with a skin adhesive 30. Skin adhesive 30. A first surface 29 of a release liner 28 is releasably adhered to skin adhesive 30, and a second surface 31 of release liner 28 faces away from skin adhesive 30.

FIG. 2 is a side cross-sectional view of a monolithic-style dermal patch (40). Monolithic dermal patch 40 includes a backing 42. A matrix 44 of skin adhesive mixed with a drug formulation (or composition) is coated on one side of backing 42. A release liner 48 is releasable adhered to matrix 44 on a surface of matrix 44 opposite the surface adhered to backing 42. First side 49 of release liner 48 faces away from matrix 44 and a portion of second side 51 of release liner 48 is adhered to matrix 44. To use the monolithic transdermal device 40, the release liner 48 is peeled away and the exposed surface of adhesive matrix 44 is applied to the skin.

FIG. 3 shows a flexible tan polyethylene, polyolefin, or polyurethane foam backing layer on a clear polyester release layer. The foam backing is the part of the patch that occludes the actives (including anti-pain drugs) from the external environment. The foam backing is the part of the patch that is placed on the skin. The tan foam backing is the actual “patch” that will be placed on the skin, and the release liner is the clear plastic sheet surrounding it that is discarded from which the patch is peeled off just before usage.

### DETAILED DESCRIPTION

As used herein, including the appended claims, the singular forms of words such as “a,” “an,” and “the” include their corresponding plural references unless the context clearly dictates otherwise. All references cited herein are incorporated by reference to the same extent as if each individual patent, and published patent application, as well as figures, drawings, sequence listings, compact discs, and the like, was specifically and individually indicated to be incorporated by reference. The present invention is not to be limited by compositions, reagents, methods, diagnostics, laboratory data, and the like, of the present disclosure. Also, the present invention is not limited by any preferred embodiments that are disclosed herein.

Menstruation (Physiology and Medical Conditions)

Conditions associated with dysmenorrhea. Dysmenorrhea occurs as primary dysmenorrhea (PD), which appears not to be caused by any pathological pelvic diseases, and as secondary dysmenorrhea (SD), which is caused by a pathological pelvic disease. Secondary dysmenorrhea can be caused by, for example, endometriosis, polycystic ovarian syndrome (PCOS), pelvic inflammatory disease, ovarian cysts, adenomyosis, uterine myomas, uterine polyps, intra-uterine adhesions, and cervical stenosis. Secondary dysmenorrhea can also be caused by intra-uterine devices. See, e.g., Rosenwaks and Seegar-Jones (1980) J. Reprod. Med. 25:207-212; Coco (1999) Am. Fam. Physican. 60:489-496; Iglesias and Coupey (1999)10:255-273; Schroeder and Sanfilippo (1999) Pediatr. Clin. North Am. 46:555-571; and Duran (2004) J. Midwifery Womens Health. 49:520-528. Dysmenorrhea can also impair the quality of life, where this impairment takes the form of mood disorders, sleep disturbances, impaired social life, or limitations in the ability to perform school work or to perform employment-related work.

Endometriosis and polycystic ovarian syndrome. Systems, formulations, dermal patches comprising a formulation, and related methods, of the present disclosure are intended for treating the symptoms of dysmenorrhea, including dysmenorrhea associated with endometriosis, with polycystic ovarian syndrome, or with other conditions. Endometriosis and polycystic ovarian syndrome are described (see, Vercellini and Fedele (2014) Nat. Rev. Endocrinol. 10:261-275; Denny (2004) Journal of Advanced Nursing. 46:641-648; Wood (2001) New Engl. J. Med. 345:266-275; Giudice (2010) New Engl. J. Med. 362:2389-2398; Sheehan (2004) Clinical Medicine and Research. 2:13-27; Homburg (2002) Human Reproduction. 17:2495-2499).

Ovarian cycle and uterine (menstrual) cycle. In the female reproductive cycle, there are two concurrent cycles, the ovarian cycle, and the uterine (menstrual) cycle. The ovarian cycle consists of a series of events that occur during and following oocyte maturation. The uterine cycle consists of a series of changes within the endometrium in preparation for the arrival of a fertilized ovum that will develop within the endometrium until birth. The reproductive cycle can subdivide into the menstrual phase, preovulatory phase, ovulation, and postovulatory phase. The function of the uterus in each of these phases follows. In the menstrual phase, a decline in estrogen and progesterone levels stimulates the release of prostaglandins, which results in vasoconstriction of arterioles within the uterus. The vasoconstriction eventually leads to hypoperfusion of these cells, which results in cell death. This process initiates the sloughing off of blood, fluid, and epithelial cells from the endometrial walls into the cervix and out through the vagina. In the preovulatory phase, estrogen is released into the blood, which repairs the endometrium. The endometrium undergoes other changes and doubles in thickness. During ovulation, the follicle ruptures and releases an oocyte which enters the uterine tube. In the postovulatory phase, progesterone and estrogen stimulate further growth of endometrial glands and thickening of the endometrium in preparation for the implantation of a fertilized ovum. If fertilization does not occur, progesterone and estrogen levels decline, and the menstruation stage occurs (see, Gasner and Aatsha (2021) Physiology, Uterus. StatPearls Publishing, Florida).

Follicular phase and luteal phase of the menstrual cycle. The follicular and luteal phases can be defined by day of the cycle after standardizing to a 28-day cycle using the last menstrual period and average duration of cycle information. The menstrual cycle can be divided into four intervals based on the cyclical fluctuations of the estrogen, progesterone, luteal hormone, and follicle stimulating hormone. Early follicular phase can range from Day 0 to Day 5. Late follicular phase can range from Day 6 to Day 15. Early luteal phase can span Day 16 to Day 24, whereas late luteal can span from between Day 25 to Day 28 (see, Khan and Badve (2002) Modern Pathology. 15:1348-1356).

Methods for Manufacturing Monolithic Cramp Patch of the Present Disclosure

Solvent casting method. Reagents and methods for solvent casting are described (see, Pagilla and Bakshi (2020) Int. J. Applied Pharmaceutical Sciences and Research. 5:45-53; Verma and Parvez (2014) Adv. Biological Research. 8:131-138; Kong, Cui, Wang (2019) Acta Biochimica Polonica. 66:464-467).

Manufacturing process for Remy Biosciences' Polyisobutylene (PIB)-based Monolithic Cramp Patch. The Remy Biosciences cramp patch is a drug-in-adhesive transdermal patch. The adhesive is a Polyisobutylene adhesive in heptane with or without tackifier and, when dried, comprises about 75 to about 85% of the dry patch adhesive matrix. B-caryophyllene, cannabidiol (CBD) or cannabidiolic acid (CBDA) in a crystalline isolate form, eugenol derived from cloves or other natural sources, capsaicin, and diclofenac in any combination comprises 20% of the dry patch matrix, although any penetration enhancers or tackifiers may be added and will reduce the amount of active pharmaceutical ingredients (APIs) listed above. Ascorbyl palmitate comprises about 0.05% to about 0.15% of the total matrix. Heptane is used as a solvent for the adhesive, ethanol or methanol are used as a solvent for the active ingredients and enhancers, and ethyl acetate was used as a cosolvent.

FIRST STEP. To mix, the APIs, permeation enhancers (if used), excipients, and preservatives were dissolved in ethanol or methanol then filtered through quantitative filter paper, any tackifiers (if used) were dissolved in heptane.

SECOND STEP. All the compounds, including solvents and adhesives, were mixed via ultra-high shear dispersion blade mixing.

THIRD STEP. The mixture was then degassed in a vacuum mixer to remove any residual air bubbles.

FOURTH STEP. To manufacture the patches, the solvent casting method was used, and the adhesive mixture described above was cast via knife-over-roll onto a silicone-treated polyester release liner (Fox River FRA 316) at a thickness of 0.007 inches. The solvents in the wet mixture were evaporated in an 85 degrees centigrade oven for no less than one minute. Residual solvents were well below acceptable maximums after this point and the total coat weight of the mixture was measured at approximately 50 grams per square meter (gsm). At this point, the release liner was laminated to an occlusive backing of polyolefin foam (UFP Technologies).

In thickness embodiments, the adhesive mixture described above can be cast at a thickness of, for example, 0.002 to 0.003 inches, 0.002 to 0.004 inches, 0.002 to 0.005 inches, 0.002 to 0.006 inches, 0.002 to 0.007 inches, 0.003 to 0.004 inches, 0.003 to 0.005 inches, 0.003 to 0.006 inches, 0.003 to 0.007 inches, 0.003 to 0.008 inches, 0.004 to 0.005 inches, 0.004 to 0.006 inches, 0.004 to 0.007 inches, 0.004 to 0.008 inches, 0.004 to 0.009 inches, 0.005 to 0.006 inches, 0.005 to 0.007 inches, 0.005 to 0.008 inches, 0.005 to 0.009 inches, 0.005 to 0.010 inches, 0.006 to 0.007 inches, 0.006 to 0.008 inches, 0.006 to 0.009 inches, 0.006 to 0.010 inches, 0.006 to 0.011 inches, 0.007 to 0.008 inches, 0.007 to 0.009 inches, 0.007 to 0.010 inches, 0.007 to 0.011 inches, 0.007 to 0.012 inches, 0.008 to 0.009 inches, 0.008 to 0.0010 inches, 0.008 to 0.0011 inches, 0.008 to 0.0012 inches, or 0.008 to 0.0013 inches, and so on. These are all range embodiments.

FIFTH STEP. After 24 hours, the laminated roll was kiss-cut to either 30 or 50 centimeter squared, rounded-edge rectangles, quality checked, packaged, and shipped for final distribution.

The patch includes a flexible tan polyethylene, polyolefin, or polyurethane foam backing layer on a clear polyester release liner. The present disclosure provides a method for 35 mg β-caryophyllene, 10 mg eugenol (as clove oil extract), and 0.05% capsaicin patches, wherein a human subject places one patch on the abdomen per day, removing the patch after 24 hours, wherein the patch is placed on the abdomen starting three days prior to the usual onset of menstruation, and wherein patch dosing comprises the step of cleaning the area of abdomen where pain is most intense during menstruation, removing the release liner from one patch, the step of placing the tan foam on the cleaned and dried area, adhesive side down and applying constant pressure to entire patch with palm of hand for 60 seconds, and finally the step of removing the patch after 24 hours. (The description of a dermal patch having comprised “a flexible tan polyethylene, polyolefin, or polyurethane foam backing layer on a clear polyester release liner” refers only to only a monolithic-style patch).

Manufacturing process for Remy Biosciences' Polyacrylate-based Monolithic Cramp Patch. The Remy Biosciences cramp patch is a drug-in-adhesive transdermal patch. The adhesive is a polyacrylate adhesive in heptane or ethyl acetate and, when dried, comprises between 55-85% of the dry patch adhesive matrix. B-caryophyllene, cannabidiol (CBD) or cannabidiolic acid (CBDA) in a crystalline isolate form, eugenol derived from cloves or other natural sources, capsaicin, and diclofenac in any combination comprises between 20-40% of the dry patch matrix, although any penetration enhancers or tackifiers may be added and will reduce the amount of active pharmaceutical ingredients (APIs) listed above. Ascorbyl palmitate comprises about 0.05% to about 0.15%% of the adhesive matrix. Heptane or ethyl acetate is used as a solvent for the adhesive, ethanol or methanol are used as a solvent for the active ingredients and enhancers, and ethyl acetate was used as a cosolvent if necessary.

FIRST STEP. To mix, the APIs, permeation enhancers (if used), excipients, and preservatives were dissolved in ethanol or methanol then filtered through quantitative filter paper, any tackifiers (if used) were dissolved in heptane.

SECOND STEP. All the compounds, including solvents and adhesives, were mixed via ultra-high shear dispersion blade mixing.

THIRD STEP. The mixture was then degassed in a vacuum mixer to remove any residual air bubbles.

FOURTH STEP. To manufacture the patches, the solvent casting method was used, and the adhesive mixture described above was cast via knife-over-roll onto a silicone-treated polyester release liner (Fox River FRA 316) at a thickness of 0.007 inches. The solvents in the wet mixture were evaporated in an 85 degrees centigrade oven for no less than one minute. Residual solvents were well below acceptable maximums after this point and the total coat weight of the mixture was measured at approximately 50 grams per square meter (gsm). At this point, the release liner was laminated to an occlusive backing of polyolefin foam (UFP Technologies).

In thickness embodiments, the adhesive mixture described above can be cast at a thickness of, for example, 0.002 to 0.003 inches, 0.002 to 0.004 inches, 0.002 to 0.005 inches, 0.002 to 0.006 inches, 0.002 to 0.007 inches, 0.003 to 0.004 inches, 0.003 to 0.005 inches, 0.003 to 0.006 inches, 0.003 to 0.007 inches, 0.003 to 0.008 inches, 0.004 to 0.005 inches, 0.004 to 0.006 inches, 0.004 to 0.007 inches, 0.004 to 0.008 inches, 0.004 to 0.009 inches, 0.005 to 0.006 inches, 0.005 to 0.007 inches, 0.005 to 0.008 inches, 0.005 to 0.009 inches, 0.005 to 0.010 inches, 0.006 to 0.007 inches, 0.006 to 0.008 inches, 0.006 to 0.009 inches, 0.006 to 0.010 inches, 0.006 to 0.011 inches, 0.007 to 0.008 inches, 0.007 to 0.009 inches, 0.007 to 0.010 inches, 0.007 to 0.011 inches, 0.007 to 0.012 inches, 0.008 to 0.009 inches, 0.008 to 0.0010 inches, 0.008 to 0.0011 inches, 0.008 to 0.0012 inches, or 0.008 to 0.0013 inches, and so on. These are all range embodiments.

FIFTH STEP. After 24 hours, the laminated roll was kiss-cut to either 30 or 50 centimeter squared, rounded-edge rectangles, quality checked via light table analysis, packaged, and shipped for final distribution.

The patch includes a flexible polyethylene, polyolefin, or polyurethane foam backing layer on a clear polyester release liner. The present disclosure provides a method for 35 mg β-caryophyllene, 10 mg eugenol (as clove oil extract), and 0.05% capsaicin patches, wherein a human subject places one patch on the abdomen per day, removing the patch after 24 hours, wherein the patch is placed on the abdomen starting three days prior to the usual onset of menstruation, and wherein patch dosing comprises the step of cleaning the area of abdomen where pain is most intense during menstruation, removing the release liner from one patch, the step of placing the tan foam on the cleaned and dried area, adhesive side down and applying constant pressure to entire patch with palm of hand for 60 seconds, and finally the step of removing the patch after 24 hours. (The description of a dermal patch having comprised “a flexible tan polyethylene, polyolefin, or polyurethane foam backing layer on a clear polyester release liner” refers only to only a monolithic-style patch).

Manufacturing process for Remy Biosciences' Polydimethylsiloxane-based Monolithic Cramp Patch. The Remy Biosciences cramp patch is a drug-in-adhesive transdermal patch. The adhesive is a polydimethylsiloxane adhesive in heptane or ethyl acetate and, when dried, comprises about 75% to about 85% of the dry patch adhesive matrix. B-caryophyllene, cannabidiol (CBD) or cannabidiolic acid (CBDA) in a crystalline isolate form, eugenol derived from cloves or other natural sources, capsaicin, and diclofenac in any combination comprises 20% of the dry patch matrix, although any penetration enhancers or tackifiers may be added and will reduce the amount of active pharmaceutical ingredients (APIs) listed above. Ascorbyl palmitate comprises about 0.05% to about 0.15% of the adhesive matrix. Heptane or ethyl acetate is used as a solvent for the adhesive, ethanol or methanol are used as a solvent for the active ingredients and enhancers, and ethyl acetate was used as a cosolvent if necessary.

FIRST STEP. To mix, the APIs, permeation enhancers (if used), excipients, and preservatives were dissolved in ethanol or methanol then filtered through quantitative filter paper, any tackifiers (if used) were dissolved in heptane.

SECOND STEP. All the compounds, including solvents and adhesives, were mixed via ultra-high shear dispersion blade mixing.

THIRD STEP. The mixture was then degassed in a vacuum mixer to remove any residual air bubbles.

FOURTH STEP. To manufacture the patches, the solvent casting method was used, and the adhesive mixture described above was cast via knife-over-roll onto a fluoropolymer-treated polyester release liner (3M) at a thickness of 0.010 inches. The solvents in the wet mixture were evaporated in an 85 degrees centigrade oven for no less than one minute. Residual solvents were well below acceptable maximums after this point and the total coat weight of the mixture was measured at approximately 50 grams per square meter (gsm). At this point, the release liner was laminated to an occlusive backing of polyolefin foam (UFP Technologies).

In thickness embodiments, the adhesive mixture described above can be cast at a thickness of, for example, 0.002 to 0.003 inches, 0.002 to 0.004 inches, 0.002 to 0.005 inches, 0.002 to 0.006 inches, 0.002 to 0.007 inches, 0.003 to 0.004 inches, 0.003 to 0.005 inches, 0.003 to 0.006 inches, 0.003 to 0.007 inches, 0.003 to 0.008 inches, 0.004 to 0.005 inches, 0.004 to 0.006 inches, 0.004 to 0.007 inches, 0.004 to 0.008 inches, 0.004 to 0.009 inches, 0.005 to 0.006 inches, 0.005 to 0.007 inches, 0.005 to 0.008 inches, 0.005 to 0.009 inches, 0.005 to 0.010 inches, 0.006 to 0.007 inches, 0.006 to 0.008 inches, 0.006 to 0.009 inches, 0.006 to 0.010 inches, 0.006 to 0.011 inches, 0.007 to 0.008 inches, 0.007 to 0.009 inches, 0.007 to 0.010 inches, 0.007 to 0.011 inches, 0.007 to 0.012 inches, 0.008 to 0.009 inches, 0.008 to 0.0010 inches, 0.008 to 0.0011 inches, 0.008 to 0.0012 inches, or 0.008 to 0.0013 inches, 0.009 to 0.010 inches, 0.009 to 0.0011 inches, 0.009 to 0.0012 inches, 0.009 to 0.0013 inches, or 0.009 to 0.0014 inches, 0.010 to 0.011 inches, 0.010 to 0.0012 inches, 0.010 to 0.0013 inches, 0.010 to 0.0014 inches, or 0.010 to 0.0015 inches, 0.011 to 0.012 inches, 0.011 to 0.0013 inches, 0.011 to 0.0014 inches, 0.011 to 0.0015 inches, or 0.011 to 0.0016 inches, and so on. These are all range embodiments.

FIFTH STEP. After 24 hours, the laminated roll was kiss-cut to either 30 or 50 centimeter squared, rounded-edge rectangles, QC'd via light table analysis, packaged, and shipped for final distribution.

The patch includes a flexible tan polyethylene, polyolefin, or polyurethane foam backing layer on a clear polyester release liner. The present disclosure provides a method for 35 mg β-caryophyllene, 10 mg eugenol (as clove oil extract), and 0.05% capsaicin patches, wherein a human subject places one patch on the abdomen per day, removing the patch after 24 hours, wherein the patch is placed on the abdomen starting three days prior to the usual onset of menstruation, and wherein patch dosing comprises the step of cleaning the area of abdomen where pain is most intense during menstruation, removing the release liner from one patch, the step of placing the tan foam on the cleaned and dried area, adhesive side down and applying constant pressure to entire patch with palm of hand for 60 seconds, and finally the step of removing the patch after 24 hours. (The description of a dermal patch having comprised “a flexible tan polyethylene, polyolefin, or polyurethane foam backing layer on a clear polyester release liner” refers only to only a monolithic-style patch).

Capsaicin (trans-8-methyl-N-vanillyl-6-nonenamide) is an alkaloid derived from plants of the genus Capsicum. Because capsaicin is not water-soluble, alcohols and other organic solvents can be used to solubilize capsaicin in topical preparations. Capsaicin and other anti-pain drugs can be administered orally, topically, by subcutaneous injection, or by any combination of these routes. The present disclosure provides monolithic patches and reservoir-style patches, that include a formulation, and where the formulation comprises one, two, three, four, or more drugs. In embodiments, one of these drugs is an anti-pain drug, or two of these drugs is an anti-pain drug, or three of these drugs is an anti-pain drug, or four of these drugs is an anti-pain drug.

FORMULATIONS THAT INCLUDE TWO DRUGS. In “consisting” embodiments, formulations of the present disclosure (and the only drugs in a dermal patch that includes the formulation) comprise only the following two drugs, for example, a first formulation with eugenol and capsaicin, a second formulation with capsaicin and β-caryophyllene (BCAP), a third formulation with eugenol and β-caryophyllene (BCAP), a fourth formulation with capsaicin and cannabidiol (CBD), a fifth formulation with eugenol and cannabidiol (CBD), a sixth formulation with capsaicin and cannabidiolic acid (CBDA), a seventh formulation with eugenol and cannabidiolic acid (CBDA), an eighth formulation with capsaicin and diclofenac, a seventh formulation with eugenol and diclofenac, and so on. The formulation is optionally supplemented with one or more of: penetration enhancers, a tackifier, and one or more excipients.

FORMULATIONS THAT INCLUDE THREE DRUGS. In “consisting” embodiments, formulation of the present disclosure consists of any combination of three of these drugs: (1) Eugenol, (2) Capsaicin, (3) β-caryophyllene (BCAP), (4) Cannabidiol (CBD), (5) Cannabidiolic acid (CBDA), (6) Diclofenac. In this “consisting” embodiment, the only drugs in the formulation (and the only drugs in a dermal patch that includes the formulation), are three of eugenol, capsaicin, β-caryophyllene (BCAP), cannabidiol (CBD), cannabidiolic acid (CBDA), or diclofenac. The formulation is optionally supplemented with one or more of: penetration enhancers, a tackifier, and one or more excipients.

RELATIVE WEIGHTS OF THREE DRUGS. For a formulation, composition, solution, slurry, or powder, that contains three different drugs (such as, eugenol, capsaicin, and β-caryophyllene (BCAP)), the respective relative weights can be, for example, 1.0, 1.0, 1.0, or 1.0, 1.0, 1.5, or 1.0, 1.0, 2.0, or 1.0, 1.0, 2.5, or 1.0, 1.0, 3.0, or 1.0, 1.0, 3.5, or 1.0, 1.0, 4.0, or 1.0, 1.0, 4.5, or 1.0, 1.0, 5.0, or 1.0, 1.0, 6.0, or 1.0, 1.0, 7.0, or 1.0, 1.0, 8.0, or 1.0, 1.0, 10.0. Also, the relative weights can be, 1.0, 2.0, 1.0, or 1.0, 2.0, 1.5, or 1.0, 2.0, 2.0, or 1.0, 2.0, 2.5, or 1.0, 2.0, 3.0, or 1.0, 2.0, 3.5, or 1.0, 2.0, 4.0, or 1.0, 2.0, 4.5, or 1.0, 2.0, 5.0, or 1.0, 2.0, 6.0, or 1.0, 2.0, 7.0, or 1.0, 2.0, 8.0, or 1.0, 2.0, 10.0. Alternatively, the relative weights can be, 1.0, 3.0, 1.0, or 1.0, 3.0, 1.5, or 1.0, 3.0, 2.0, or 1.0, 3.0, 2.5, or 1.0, 3.0, 3.0, or 1.0, 3.0, 3.5, or 1.0, 3.0, 4.0, or 1.0, 3.0, 4.5, or 1.0, 3.0, 5.0, or 1.0, 3.0, 6.0, or 1.0, 3.0, 7.0, or 1.0, 3.0, 8.0, or 1.0, 3.0, 10.0. In another aspect, the relative weights can be, 1.0, 4.0, 1.0, or 1.0, 4.0, 1.5, or 1.0, 4.0, 2.0, or 1.0, 4.0, 2.5, or 1.0, 4.0, 3.0, or 1.0, 4.0, 3.5, or 1.0, 4.0, 4.0, or 1.0, 4.0, 4.5, or 1.0, 4.0, 5.0, or 1.0, 4.0, 6.0, or 1.0, 4.0, 7.0, or 1.0, 4.0, 8.0, or 1.0, 4.0, 10.0. Moreover, the relative weights can be, 1.0, 5.0, 1.0, or 1.0, 5.0, 1.5, or 1.0, 5.0, 2.0, or 1.0, 5.0, 2.5, or 1.0, 5.0, 3.0, or 1.0, 5.0, 3.5, or 1.0, 5.0, 4.0, or 1.0, 5.0, 4.5, or 1.0, 5.0, 5.0, or 1.0, 5.0, 6.0, or 1.0, 5.0, 7.0, or 1.0, 5.0, 8.0, or 1.0, 5.0, 10.0, and so on.

The above values can be “exact” values, or they can be “about” values, or each of the stated values can encompass a range of plus or minus 5 percent of that value, or each of the stated values can encompass a range of plus or minus 10 percent of that value, or each of the stated values can encompass a range of plus or minus 20 percent of that value.

FORMULATIONS THAT INCLUDE FOUR DRUGS. In yet another “consisting” embodiment, formulation of the present disclosure consists of any combination of four of these drugs: (1) Eugenol, (2) Capsaicin, (3) β-caryophyllene (BCAP), (4) Cannabidiol (CBD), (5) Cannabidiolic acid (CBDA), (6) Diclofenac. In this “consisting” embodiment, the only drugs in the formulation (and the only drugs in a dermal patch that includes the formulation), are four of eugenol, capsaicin, β-caryophyllene (BCAP), cannabidiol (CBD), cannabidiolic acid (CBDA), or diclofenac. The formulation is optionally supplemented with one or more of: penetration enhancers, a tackifier, and one or more excipients.

SALTS. The present disclosure provides chemicals that include one of the following counterions. These counterions are magnesium, potassium, sodium, and zinc. In exclusionary embodiments, systems, dermal patches, formulations, dermal patches comprising a formulation, of the present disclosure can exclude any system, dermal patch, formulation, or dermal patch comprising a formulation that includes one or more of the above counterions.

CONCENTRATION EMBODIMENTS. In one or more of the above-disclosed formulations, any one particular drug can be present at a concentration of 0.015%, 0.020%, 0.025%, 0.050%, 0.075%, 0.10%, 0.125%, 0.150%, 0.175%, 0.20%, 0.30%, 0.40%, 0.50%, 1.0%, 2.0%, 4.0%, 6.0%, 8.0%, 10%, 12%, 14%, 16%, 18%, 20%, 22%, 24%, 26%, 28%, 30%, 32%, 34%, 36%, 38%, or 40%, by weight.

Also, in one or more of the above formulations, any one particular drug can be present at a concentration of about 0.015%, about 0.020%, about 0.025%, about 0.050%, about 0.075%, about 0.10%, about 0.125%, about 0.150%, about 0.175%, about 0.20%, about 0.30%, about 0.40%, about 0.50%, about 1.0%, about 2.0%, about 4.0%, about 6.0%, about 8.0%, about 10%, about 12%, about 14%, about 16%, about 18%, about 20%, about 22%, about 24%, about 26%, about 28%, about 30%, about 32%, about 34%, about 36%, about 38%, or about 40%, by weight.

ADDITIONAL CHEMICALS (chemicals other than anti-pain drugs). In embodiments, additional chemicals can include an emulsifier such as macrogol cetostearyl ether, a permeation enhancer, such as oleyl alcohol, a gelling agent such as carbomer, a solvent such as one or more of water, isopropyl alcohol, and propylene glycol, a fragrant chemical such as a terpene or a combination of two or more terpenes, a preservative such as butylated hydroxytoluene (BHT), and an emollient such as liquid paraffin or cocoyl caprylocaprate.

ADDITIONAL CHEMICALS (additional chemicals that are anti-pain drugs).

Dermal patches of the present disclosure can contain a formulation that comprises one or more of, pregabalin, duloxetine, gabapentin, resiniferatoxin (RTX), rutaecarpine, capsazepine, ketoprofen, etofenamate, or flufenamic acid. In other embodiments, the formulation can include a non-steroidal anti-inflammatory drug (NSAID) that is not mentioned above.

Exclusionary embodiments. In exclusionary embodiments, systems, dermal patches, formulations, and related methods, can exclude any system, can exclude any dermal patch, can exclude any formulations, and can exclude any formulation that comprises one or more of the above anti-pain drugs.

Capsazepine is a TRPV1 antagonist, and it is a potential alternative to capsaicin. Ibuprofen is a cyclooxygenase (COX) inhibitor. Ibuprofen is a non-opioid analgesic and NSAID. Diclofenac inhibits prostaglandin synthesis by inhibiting COX-1 and COX-2. Diclofenac may also inhibit the thromboxane-prostanoid receptor, and it may also inhibit lipoxygenase enzymes, and it may also activate the nitric oxide/(C)GMP antinociceptive pathway

“ABOUT” EMBODIMENTS. Without implying any limitation, the term “about” can mean a range that comprises the stated concentration where the upper limit of the range is the sum of the stated concentration plus five percent that stated concentration, and where the lower limit of the range is the stated concentration minus five percent of the stated concentration.

Without implying any limitation, the term “about” can mean a range that comprises the stated concentration where the upper limit of the range is the sum of the stated concentration plus ten percent that stated concentration, and where the lower limit of the range is the stated concentration minus ten percent of the stated concentration.

“RANGE” EMBODIMENTS. In one or more of the above formulations, any one particular drug can be present in a concentration range, such as, 0.01-0.04%, 0.01-0.08%, or 0.01-0.10%, 0.02-0.04%, 0.02-0.08%, 0.02-1.0%, or 0.02-1.2%, 0.04-0.06%, 0.04-0.08%, 0.04-0.10%, 0.04-0.12%, or 0.04-0.14%, 0.06-0.08%, 0.06-0.10%, 0.06-0.12%, 0.06-0.14%, 0.06-0.16%, or 0.06-0.18%, 0.08-0.10%, 0.08-0.14%, 0.08-0.18%, 0.08-0.20%, or 0.08-0.22%, 0.1-0.12%, 0.1-0.14%, 0.1-0.16%, 0.1-0.18%, 0.1-0.20%, or 0.1-0.22%, 0.2-0.22%, 0.2-0.24%, 0.2-0.26%, 0.2-0.28%, 0.2-0.30%, or 0.2-0.32%.

EXCLUSIONARY EMBODIMENTS. In exclusionary embodiments, a formulation of the present disclosure can exclude any formulation, can exclude any dermal patch that comprises a formulation, where the formulation includes only one drug and where this one drug occurs at any of the above concentrations, or where this one drug occurs within any of the above concentration ranges.

In exclusionary embodiments, a formulation of the present disclosure can exclude any formulation, can exclude any dermal patch that comprises a formulation, where the formulation includes two or more drugs and where at least one of these two or more drugs occurs at any of the above concentrations, or where this one of these two or more drugs occurs within any of the above concentration ranges.

In exclusionary embodiments, a dermal patch of the present disclosure that comprises a formulation of the present disclosure can exclude any formulation, and can exclude any dermal patch that comprises a formulation, where the formulation includes only one drug and where this only one drug occurs at any of the above concentrations, or where this only one drug occurs within any of the above concentration ranges.

In exclusionary embodiments, a dermal patch of the present disclosure that comprises a formulation of the present disclosure can exclude any formulation, and can exclude any dermal patch that comprises a formulation, where the formulation includes two or more drugs and where one of these two or more drugs occurs at any of the above concentrations, or where this one of these two or more drugs occurs within any of the above concentration ranges.

In exclusionary embodiments relating to enhancers, tackifiers, and excipients, a dermal patch of the present disclosure that comprises a formulation of the present disclosure can exclude any formulation, and can exclude any dermal patch that comprises a formulation, where the formulation includes one or more penetration enhancers, a tackifier, and one or more excipients.

In exclusionary embodiments, systems, dermal patches, formulations, gels, and methods of the present disclosure can exclude any system, dermal patch, formulation, gel, and method that is used for (or comprises) oral administration of one or more anti-pain drugs, that is used for (or comprises) intravenous administration of one or more anti-pain drugs, that is used for (or comprises) subdermal injection of one or more anti-pain drugs, that is used for sublingual administration of one or more anti-pain drugs, or that is used for mucosal administration (e.g., oral or rectal) administration of one or more anti-pain drugs.

Manufacturing Monolithic-Style Dermal Patches

Manufacturing Method for Monolithic Patches. Without implying any limitation, in methods of manufacturing embodiments, monolithic patches can be made as follows. Active ingredients, such as one or more anti-pain drugs, can be combined with permeation enhancer only, combined with carrier only, or combined with both permeation enhancer and carrier. Carrier can comprise, for example, one or more of oleic acid and dodecyl methyl sulfoxide. What also can be added and combined include one or more permeation enhancers, one or more antioxidants, one or more preservatives, and one or more terpenes. Then, a polymer such as a silicone polymer is mixed in. Finally, the mixture is spread into one or more sheets and dried in an oven until solvents have evaporated. After drying, a foam backing layer is applied, and then the product is cut into shapes (e.g., squares, rectangles, ovals, round-edged squares or round-edged rectangles, circles) suitable for applying to the skin of a person.

Adhesive mix is dispensed on the release liner by means of “knife-over-roll” coating method and dried in the oven at drying time from 1 min to 3 min or until all residual solvents are below acceptable maximums, for example, 100 ppm, 10 ppm, 1 ppm, or other acceptable maximums. Dried adhesive film is laminated to the backing film and edges are slit for further die cutting of the patches. The laminate is placed on the die cutting machine and proper size patches are cut and later packaged into pouches and boxes.

Monolithic devices may also include one or more penetration enhancers, including azone, oleic acid, dihydromyricetin, limonene, dimethylsulfoxide (DMSO), 1,2-propylene glycol, 2-(2-Ethoxyethoxy)ethanol (Transcutol®), isopentenyl pyrophosphate (IPP), or isopropyl myristate. The amount of penetration enhancer preferably ranges from zero (0) to about ten (10) percent by weight of the matrix.

The skin contact area of the device is preferably at least about 10 square centimeters, more preferably at least about 20 square centimeters, and still more preferably at least about 30 square centimeters. At the same time, the skin contact area of the device is preferably no more than about 70 square centimeters, preferably no more than about 60 square centimeters, and still more preferably no more than about 50 square centimeters.

Knife-Over-Roll Coating. Knife coating is a process by which a thin liquid coating is formed on a continuous web by the application of an excess of coating liquid which is subsequently metered by a rigid knife held in close proximity to a rigidly supported web. The thickness of the coating depends primarily on the clearance, or gap, between the knife and the web, and upon the geometry of the gap (bevel angle, length). Roll coating is a process by which a thin liquid film is formed on a continuous web by use of two or more rotating rolls, such that the fluid flow in a small gap between a pair of rotating rolls is the primary factor controlling the thickness and uniformity of the coated film. The thickness of the coating depends primarily on the gap between adjacent rolls and their relative speeds. Two basic types of roll coaters are distinguished by the relative direction of roll surface motion in the gap: in forward roll coating the roll surfaces move in the same direction and in reverse roll coating they move in opposite directions. In terms of the flow fields, knife coating is a subset of forward roll coating where one surface is stationary. See, Coyle, D. J (1997) Knife and Roll Coating in Liquid Film Coating (ed. S. F. Kistler and P. M. Schweizer). Chapman & Hall, London; W. Rehnby, M. Gustafsson, M. Skrifvars (June 2008) Conference Paper, Coating of Textile Fabrics with Conductive Polymers for Smart Textile Applications, pages 100-103.

Suitable examples of such amine-compatible silicone adhesives include the BIO-PSA 7-4301 and 7-4302 skin adhesives supplied by Dow Corning. BIO-PSA 7-4301 is a high tack, amine-compatible silicone adhesive in heptane available with a solids content of 60 percent to 70 percent and corresponding viscosities at 20 degrees C. of 450 mPa-s and 1600 mPa-s. BIO-PSA 7-4302 is a high tack, amine-compatible silicone adhesive in ethyl acetate with a solids content of 60 percent by weight and a viscosity of 1200 mPa-s at 20 degrees C. The skin adhesive is coated to a thickness per unit area on the membrane that, when dry, is preferably from about 10 to about 80 g/square meters, more preferably from about 40-70 g/square meter, and still more preferably from about 45-60 g/square meter.

Suitable examples of such polyisobutylene adhesives include custom formulated polyisobutylene adhesives made up of a mixture of low, medium, and high molecular weight OPPANOL® polyisobutylenes, available from BASF, either with or without a tackifier in the form of a fully or partially hydrogenated hydrocarbon resin, such as Escorez™, available by ExxonMobil Chemical, or equivalent. In this embodiment, “low molecular weight polyisobutylenes” are defined as polyisobutylenes with an average molecular weight under about 100,000 grams per mole (viscosity average). In this embodiment, “medium molecular weight polyisobutylenes” are defined as polyisobutylenes with an average molecular weight between about 100,000 and about 350,000 grams per mole (viscosity average). In this embodiment, “high molecular weight polyisobutylenes” are defined as polyisobutylenes with an average molecular weight over about 350,000 grams per mole (viscosity average). Suitable examples of such polyisobutylene adhesives may also include the DURO-TAK 87-6908 skin adhesive provided by Henkel. DURO-TAK 87-6908 is a low tack, medium peel adhesion, low shear resistance polyisobutylene adhesive in n-heptane, available with a solids content of about 38% and a corresponding viscosity at 20 degrees centigrade of about 6000 cP. The skin adhesive is coated to a thickness per unit area on the membrane that, when dry, is preferably from about 10 to about 60 g/square meters, more preferably from about 35-55 g/square meter, and still more preferably from about 40-50 g/square meter.

Suitable examples of such polyacrylate adhesives include the DURO-TAK 87-2074, 87-2194, and 87-2852 skin adhesives supplied by Henkel. DURO-TAK 87-2074 is a crosslinked, medium tack, medium peel adhesion, high shear resistance polyacrylate adhesive in ethyl acetate with —COOH/—OH functional groups that does not contain vinyl acetate, available with a solids content of about 29.5% and corresponding viscosity at 20 degrees Centigrade of about 1500 cPs. DURO-TAK 87-2194 is a crosslinked, low tack, medium peel adhesion, high shear resistance polyacrylate adhesive in ethyl acetate with —COOH functional groups that contains vinyl acetate, available with a solids content of about 45% and corresponding viscosity at 20 degrees Centigrade of about 3000 cP. DURO-TAK 87-2852 is a crosslinked, medium tack, medium peel adhesion, high shear resistance polyacrylate adhesive in ethyl acetate with —COOH functional groups that does not contain vinyl acetate, available with a solids content of about 33.5% and corresponding viscosity at 20 degrees Centigrade of about 2500 cP. The skin adhesive is coated to a thickness per unit area on the membrane that, when dry, is preferably from about 10 to about 60 g/square meters, more preferably from about 35-55 g/square meter, and still more preferably from about 40-50 g/square meter.

TIME TO PAIN RELIEF. In embodiments, systems, dermal patches, formulations, gels, and methods of the present disclosure provide a “time to pain relief” of under 120 minutes, under 100 min., under 80 min., under 60 min., under 50 min., under 40 min., under 30 min., under 25 min., under 20 min, under 15 min., or under 10 min. The parameter, “time to pain relief” can be defined, for example, as 80 percent or less than a defined maximum amount of pain, as 70 percent or less, as 60 percent or less, as 50 percent or less, as 40 percent or less, as 30 percent or less, as 25 percent or less, as 20 percent or less, or as 15 percent or less than a defined maximal amount of pain. Alternatively, “time to pain relief” can be defined as “the time to significant pain relief.”

### EXAMPLES

Patch comprising a flexible tan polyethylene, polyolefin, or polyurethane foam backing layer on a clear polyester release layer. The following concerns patches of the present disclosure, where the patch comprises the anti-pain drugs eugenol, capsaicin, and β-caryophyllene, or where the patch comprises the anti-pain drugs eugenol, capsaicin, and cannabidiol (CBD), or where the patch comprises the anti-pain drugs eugenol, capsaicin, and cannabidiolic acid (CBDA), or where the patch comprises the anti-pain drugs eugenol, capsaicin, and diclofenac. The foam backing is the part of the patch that occludes the actives from the external environment. It is the part of the patch that is placed on the skin. The tan foam backing is the actual “patch” that will be placed on the skin, and the release liner is the clear plastic sheet surrounding it that is discarded from which the patch is peeled off just before usage. (The description of a dermal patch having comprised “a flexible tan polyethylene, polyolefin, or polyurethane foam backing layer on a clear polyester release liner” refers only to only a monolithic-style patch.)

Drugs that can be omitted from the claims, or drugs that can be excluded from the claims by way of a negative claim limitation. Drugs that are omittable or and drugs that are excludable include, opioids, muscle relaxants, and corticosteroids, as well as anti-anxiety, anti-depressant, and anticonvulsant drugs with anti-pain effects and uses.

Adhesives, antioxidants, tackifiers, and permeation enhancers. Formulations, compositions, and dermal patches comprising said formulations or compositions, may comprise polyisobutylene adhesive (PIB), ascorbyl palmitate as an antioxidant for the CBD, an antioxidant from the list of: butylated hydroxytoluene (BHT), butylated hydroxyanisole (BHA), Tocopherol, ascorbyl palmitate, propyl gallate, ascorbic acid, or citric acid to avoid oxidation of any excipients, a tackifier in the form of a resin, such as the Escorez® family by Exxon Mobil®, the HF and CH family by TeckRez, and the Arkon® family by Arakawa®, as well as a permeation enhancer such as azone, oleic acid, dihydromyricetin, limonene, dimethylsulfoxide (DMSO), 1,2-propylene glycol, 2-(2-Ethoxyethoxy)ethanol (Transcutol®), isopentenyl pyrophosphate (IPP), or isopropyl myristate. The chemical structure of transcutol has been described as diethylene glycol monoethyl ether (see, Osborne and Musakhanian (2018) Advances in Topical Delivery of Drugs. 19:3512-3533; Lamas, Villaggi, Nocito (2006) Int. J. Pharm. 307:239-243); Mura, Faucci, Bramanti (2000) European J. Pharmaceutical Sciences. 9:365-372.

Pain scores for measuring menstrual pain, and for measuring health related quality of life before and after treatment for menstrual pain. Efficacy of systems, dermal patches, formulations, gels, and methods of the present disclosure can be assessed by the following scales and scoring systems:

The change from baseline of Visual Analogue Scale (VAS)-interpreted pain during menstruation. The VAS is a psychometric response scale designed to measure a characteristic (in this case, pain) that is distributed over a continuum of values and is not directly measurable (see, Younger, McCue, Mackey (2009) Pain outcomes: a brief review of instruments and techniques. Curr Pain Headache Rep. Vol. 13:39-43). The VAS is comprised of a 10 cm line that ranges from “no pain” on the left (0 cm) to “worst pain” on the right (10 cm). Subjects are asked to record pain at any point along the line, and the measurement from 0 cm to the participant's mark, recorded in cm, is interpreted as their pain.

The change from baseline of Cox Menstrual Symptom Scale (CMSS)-interpreted menstruation symptoms, including cramp and abdominal pain frequency and severity. The CMSS includes 18 symptoms, each evaluated for frequency and severity over 5 levels. On the frequency scale, a score of 0 denotes that the symptom “did not occur,” a 1 denotes that it “lasted less than 3 hours;” a 2 that it “lasted 3-7 hours;” a 3 that it “lasted an entire day;” and a 4 that it “lasted several days.” On the severity scale, a score of 0 denotes that the symptom was “not noticeable;” a 1 that it was “slightly bothersome;” a 2 that it was “moderately bothersome;” a 3 that it was “severely bothersome;” and a 4 that it was “very severely bothersome.” The CMSS also includes an additional question: “How much additional time did you spend in bed because of menstrual problems over the duration of your last period? Give estimated total number of hours as: [fill this out] hours.”

The change from baseline of Quality of Life Scale (QOLS)-interpreted quality of life. The Flanagan QOLS seeks to assess Quality of Life (QOL) in patients with chronic illness. QOL measurement is especially meaningful to determine the impact and efficacy of treatment when a cure is not possible (see, Younger, McCue, Mackey (2009) Pain outcomes: a brief review of instruments and techniques., Curr Pain Headache Rep. Vol. 13:39-43). The Flanagan QOLS is usually considered to be one of the only QOL measurements that quantifies true QOL rather than causal indicators of QOL. The QOLS includes 16 categories that are distributed over five conceptual domains: (1) Physical and material well-being; (2) Relations with other people; (3) Social, community, and civic activities; (4) Personal development and fulfillment; and (5) Recreation. Each of the 16 categories within the five conceptual domains (see above) are measured on a seven point scale: Terrible (one point), Unhappy (two points), Mostly dissatisfied (three points), Mixed (four points), Mostly satisfied (five points), Pleased (six points), and Delighted (seven points) (see, Bahmani M et al. (2015) Effect of iranian herbal medicines in dysmenorrhea phytotherapy., J Chem Pharm. 519-526). The categories are defined as follows:

PHYSICAL AND MATERIAL WELL-BEING (categories one and two). Material comforts—things like a desirable home, good food, possessions, conveniences, and increasing income, and security for the future. Health and personal safety—to be physically fit and vigorous, to be free from anxiety and distress, and to avoid bodily harm;

RELATIONS WITH OTHER PEOPLE (categories three through six). Relationships with your parents, brothers, sisters, and other relatives—things like communicating, visiting, understanding, doing things, and helping and being helped by them. Having and raising children—this involves being a parent and helping, teaching, and caring for your children. Close relationship with a husband or wife or partner—An intimate relationship. Close friends—sharing activities, interests, and views; being accepted, visiting, giving and receiving help, love trust, support, guidance.

SOCIAL, COMMUNITY, AND CIVIC ACTIVITIES (categories seven and eight). Helping and encouraging others—this includes adults or children other than relatives or close friends. These can be your own efforts or efforts as a member of a religious group, a club, or a volunteer group. Participation in activities relating to local and national government and public affairs.

PERSONAL DEVELOPMENT AND FULFILLMENT (categories nine through thirteen). Learning—attending school, improving your understanding, or getting additional knowledge. Understanding yourself and knowing your assets and limitations, knowing what life is all about, and making decisions on major life activities. For some people, this includes religious or spiritual experiences. For others, it is an attitude towards life or a philosophy. Work in a job at home that is interesting, rewarding, worthwhile. Expressing yourself in a creative manner in music, art, writing, photography, practical activities, or leisure-time activities. Independence, the ability to do for yourself.

RECREATION (categories fourteen through sixteen). Socializing—meeting other people, doing things with them, and giving or attending parties. Reading, listening to music, or observing sporting events or entertainment. Participation in active recreation—such as sports, traveling and sightseeing, playing games, singing, dancing, playing an instrument, acting, and other such activities.

The subject is asked to fill out their answers with the following instructions: “Please read each item and circle the number that best describes how satisfied you are at this time. Please answer each item even if you do not currently participate in an activity or have a relationship. You can be satisfied or dissatisfied with not doing the activity or having a relationship.”

Visual Analogue Scale (VAS) (see, Kelly (2001)); see, De Sanctis et al. (2016) Rivista Italiana di Medicina dell Adolescenza. Vol. 14 (11 pages).

Verbal Multidimensional Scoring system (VMS) (see, De Sanctis et al. (2016) Rivista Italiana di Medicina dell Adolescenza. Vol. 14 (11 pages).

Numeric Rating Scale (NRS), 7-point NRS, 11-point NRS (see, Breivik, Allen, Stubhaug (2008) Brit. J. Anaesthesia. Vol. 101:17-24; see, Wade, Crawford, Brown (2019) J. Int. Medical Research. 4454-4468)).

Pittsburgh Sleep Quality Index (see, Iacovides, Avidon, Baker (2009) Sleep. Vol. 32:1019-1026).

Penn Daily Symptom Rating Form (see, Iacovides, Avidon, Baker (2009) Sleep. Vol. 32:1019-1026, which cites Freeman, Rickels (1996) Psychiatry Res. 65: 97-106).

Polysomnographic (PSG) Sleep Variables (these include, TIB, TST, and SOL) (see, Iacovides, Avidon, Baker (2009) Sleep. Vol. 32:1019-1026).

Brief Pain Inventory (BPI) (see, Daut, Cleeland, Flannery (1983) Development of the Wisconsin Brief Pain Questionnaire. Pain. 17:197-210; see, Breivik, Allen, Stubhaug (2008) Brit. J. Anaesthesia. Vol. 101:17-24).

Profile of Mood States (POMS) Questionnaire (see, Iacovides, Avidon, Baker (2009) Sleep. Vol. 32:1019-1026, which cites McNair, Lorr, Droppleman (1992) EdITS manual for the profile of mood states. Educational and Industrial Testing Service, San Diego, CA.

McGill Pain Questionnaire (MPQ) (see, Breivik, Allen, Stubhaug (2008) Brit. J. Anaesthesia. Vol. 101:17-24).

Massachusetts General Hospital Pain Center's Pain Assessment Form. (see, Breivik, Allen, Stubhaug (2008) Brit. J. Anaesthesia. Vol. 101:17-24; see, LeBel (2006) Assessment of Pain in Ballantyne (2006) The Massachusetts General Hospital Handbook of Pain Assessment, pp. 58-75).

SF-36. The SF-36 is another scoring tool for assessing pain, and consequences of pain (mental or physical consequences). SF-36 is a short form with 36 questions, intended to be filled out by a patient. SF-36 can be used for assessing Health Related Quality of Life (HRQoL). According to a book about clinical trials intended for testing efficacy and safety of new drugs, “SF-36 questions fall into eight domains: physical function, physical role limitations, vitality, general health perceptions, pain, social function, emotional role limitations, and mental health. The domain of physical function has 10 items (10 questions) that capture, for example, the ability to carry groceries, walk, climb stairs, and dress oneself. The domain of pain has two items, which are concerned with the amount of pain experienced in the previous 4 weeks, and the extent to which pain interferes with normal work activities. The domain of mental health has five items, which concern anxiety and depression. The domain scores are then used to calculate summary scores. The SF-36 tool has two summary scores, physical summary score and mental summary score” (see, pages 451-482 of Tom Brody (2016) Clinical Trials, 2nd ed., Elsevier, San Diego, CA).

Cannabinoids

Formulations can include one or more cannabinoids. Major cannabinoids from Cannabis sativa are cannabigerolic acid (CBGA), delta-9-tetrahydrocannabinolic acid (delta-9-THCA), cannabidiolic acid (CBDA), cannabichromenic acid (CBCA), cannabigerovarinic acid (CBGVA), tetrahydrocannabivarinic acid (THCVA), cannabidivarinic acid (CBDVA), cannabichromevarinic acid (CBCVA). These major cannabinoids can be decarboxylated to form neutral plant cannabinoids, such as cannabigerol (CBG), delta-9-tetrahydrocannabinol (delta-9-THC), cannabidiol (CBD), cannabichromene (CBC), and their propyl analogues: cannabigerovarin (CBGV), tetrahydrocannabivarin (THCV), cannabidivarin (CBDV), and cannabichromevarin (CBCV). Formulations can further include cannabinol (CBN), the nonenzymatic oxidation byproduct of tetrahydrocannabinol (THC), as well as delta-8-tetrahydrocannabinol (delta-8-THC), delta-10-tetrahydrocannabinol (delta-10-THC), and other cannabinoids.

One of more of the following cannabinoids can be included in the compositions of the present disclosure. Cannabinoids and related compounds further include, for example, cannabichromene; cannabitriol; cannabicyclolol; cannabielsoin, cannabinodiol; delta-8-tetrahydrocannabinol; cannabichromanone; cannabicoumaronone; cannabicitran; 10-oxo-delta6a10a-tetrahydrocannabinol; cannabiglendol; delta-7-isotetrahydrocannabinol; CBLVA; CBV; CBEVA-B; CBCVA; delta-9-THCVA; CBDVA; CBGVA; divarinolic acid; quercetin; kaemferol; dihydrokaempferol; dihydroquercetin; cannflavin B; isovitexin; apigenin; naringenin; eriodictyol; luteolin; orientin; cytisoside; vitexin; canniprene; 3,4′-dihydroxy-5-methoxy bibenzyl; dihydroresveratrol; 3,4′-dihydroxy-5,3′-dimethoxy-5′-isoprenyl; cannabistilbene 1; cannabistilbene 11a; cannabistilbene 11b; cannithrene 1; cannithrene 2; cannabispirone; isocannabispirone; cannabispirenon-A; cannabispirenone-B; cannabispiradienone; alphacannabispiranol; beta-cannabispiranol; acetyl-cannabispirol; 7-hydroxy-5-methoxyindan-1-spirocyclohexane; 5-hydroxy-7-methoxyindan-1-spiro cyclohexane; myristic acid, palmitic acid, oleic acid, stearic acid, linoleic acid, linolenic acid, arachidic acid, eicosenoic acid, behenic acid, lignoceric acid, 5,7-dihydroxyindan-1-cyclohexane; cannabispiradienone; 3,4′-dihydroxy-5-methoxybibenzyl; canniprene; cannabispirone; cannithrene I; cannithrene 2; alphacannabispiranol; acetyl-cannabispirol; vomifoliol; dihydrovomifoliol; beta-ionone; dihydroactinidiolide; palustrine; palustridine; plus-cannabisativine; anhydrocannabisativine; dihydroperiphylline; cannabisin-A; cannabisin-B; cannabisin-C; cannabisin-D; grossamide; cannabisin-E; cannabisin-F; cannabisin-G; and so on (see, e.g., Flores-Sanchez and Verpoorte (2008) Secondary metabolism in cannabis. Phytochem. Rev. 7:615-639).

In exclusionary embodiments, systems, devices, formulations, compositions, and methods of the present disclosure can exclude any system, device, formulation, composition, or method that comprises CBD, CBC, CBG, delta-9-THC, CBN, or any chemical in the above list.

### Matrix Embodiments

An excipient useful for granulating agents and sprays is the polyvinylpyrrolidone copolymer having a given ratio, or range of ratios, of polyvinylpyrrolidone/vinyl acetate (PVP/VA). The present disclosure provides PVP/VA (or combinations of any two polymers), at a ratio of 10/90, 20/80, 30/70, 40/60, 50/50, 60/40, 70/30, 80/20, 90/10, as well as a combination of any two polymer at a ratio of about 10/90, about 20/80, about 30/70, about 40/60, about 50/50, about 60/40, about 70/30, about 80/20, about 90/10. Also, the present disclosure can exclude PVP/VA compositions (or it can exclude a combination of any two polymers) with a ratio of, 10/90, 20/80, 30/70, 40/60, 50/50, 60/40, 70/30, 80/20, 90/10, or about 10/90, about 20/80, about 30/70, about 40/60, about 50/50, about 60/40, about 70/30, about 80/20, about 90/10, and the like. The PVP/VA copolymer has the ability to distribute homogeneously around an active ingredient during formation of an aqueous liquid phase (see, US2016/0058866 of Sekura). Polymers and copolymers are available from Sigma-Aldrich, St. Louis, MO, Nippon Shokubai Co., Ltd., Osaka, Japan, BASF Corp., Florham Park, N J, and Ashland, Schaffhausen, Switzerland.

In methods of manufacturing embodiments, a monolithic patch can be made as follows. One or more drugs that relieve pain, such as diclofenac, capsaicin, cannabidiol (CBD), β-caryophyllene, eugenol, or cannabidiolic acid (CBDa) can be combined with permeation enhancer only, combined with carrier only, or combined with both permeation enhancer and carrier. Carrier can comprise, for example, one or more of oleic acid and dodecyl methyl sulfoxide. Optionally, one or more pure terpenes, or an essential oil, or a combination of an essential oil and one or more pure terpenes, is mixed with the above combination. Then, a polymer such as a silicone polymer is mixed in. Finally, the mixture is spread into one or more sheets and dried in an oven. After drying, a foam backing layer is applied, and then the product is cut into shapes (e.g., squares, rectangles, ovals, round-edged squares or round-edged rectangles, circles) suitable for applying to the skin of a person.

As stated above, the description of a dermal patch having comprised “a flexible tan polyethylene, polyolefin, or polyurethane foam backing layer on a clear polyester release liner,” refers only to only a monolithic-style patch.

Tackifiers

The present disclosure provides compositions, patches, and methods that encompass one or more of Escorez 1000 Series-aliphatic resins; Escorez 2000 Series-aromatic modified aliphatic resins; Escorez 5300 Series-water white hydrogenated cycloaliphatic resins; Escorez 5400 Series-light color hydrogenated cycloaliphatic resins; Escorez 5600 Series-light color hydrogenated aromatic modified cycloaliphatic resins; Escorene® Ultra ethylene vinyl acetate (EVA) copolymers; ExxonMobil® ethylene n-butyl acrylate (EnBA) copolymers; Optema® EMA (ethyl methyl acrylate) resins (ExxonMobil, Inc.). We also use the term “Tackifier” to mean any low molecular-weight oligomer, not of the resin type, that increases the tack of the system. One example includes, but is not limited to, a class of very low molecular-weight polyisobutylene compounds, low molecular weight polybutene compounds, or other natural oils or resins not of the aliphatic type that when compounded with the adhesive, soften it and therefore increase the tack.

Escorez® 5400 is a hydrocarbon polymer additive available from ExxonMobil Chemical Company. It has a softening point of 103° C., a weight average molecular weight of about 400 g/mole, and a dicyclopentadiene/cyclopentadiene/methylcyclopentadiene content of 40-80 wt % (see, WO2013/176712 of Block).

Escorez® 5415 is a hydrocarbon polymer additive available from ExxonMobil Chemical Company. It has a softening point of 118° C., a weight average molecular weight of about 430 g/mole, and a dicyclopentadiene/cyclopentadiene/methylcyclopentadiene content of 40-80 wt % (see, WO2013/176712 of Block).

Escorez® 5340 is a hydrocarbon polymer additive available from ExxonMobil Chemical Company. It has a softening point of 140° C., a weight average molecular weight of about 460 g/mole, and a dicyclopentadiene/cyclopentadiene/methylcyclopentadiene content of 40-80 wt % (see, WO2013/176712 of Block).

Escorez® 5600 is a hydrocarbon polymer additive available from ExxonMobil Chemical Company. It has a softening point of 103° C., a weight average molecular weight of about 520 g/mole, and a dicyclopentadiene/cyclopentadiene/methylcyclopentadiene content of 40-80 wt % (see, WO2013/176712 of Block).

Escorez® 5615 is a hydrocarbon polymer additive available from ExxonMobil Chemical Company. It has a softening point of 118° C., a weight average molecular weight of about 500 g/mole, and a dicyclopentadiene/cyclopentadiene/methylcyclopentadiene content of 40-80 wt % (see, WO2013/176712 of Block).

The present disclosure also provides compositions, patches, and methods that encompass one or more of Teckrez HF-100 series fully hydrogenated C9 water-white hydrocarbon resins and Teckrez CH-1100 series fully hydrogenated hydrocarbon resins (Teckrez, Inc).

Teckrez HF-100 is a hydrocarbon polymer additive available from Teckrez, Inc. It has a softening point of 100° C., a weight average molecular weight of about 625 g/mole, and a melt viscosity at 125° C. of about 6000 cP.

Teckrez CH-1100 is a hydrocarbon polymer additive available from Teckrez, Inc. It has a softening point of 100° C., a weight average molecular weight of about 800 g/mole, and a melt viscosity at 125° C. of about 800 cP.

The present disclosure also provides compositions, patches, and methods that encompass the Arkon™ P-100 series of hydrogenated water-white hydrocarbon resin (ARAKAWA CHEMICAL INDUSTRIES, LTD.)

ARKON™ P-100 is a hydrocarbon polymer additive available from ARAKAWA CHEMICAL INDUSTRIES, LTD. It has a softening point of 100° C., a weight average molecular weight of about 850 g/mole, and a melt viscosity at 150° C. of about 2900 cP.

Hydrogels

Hydrogels are 3-dimensional, cross-linked networks of water-soluble polymers. The porous structure of hydrogels can be altered by changing the density of cross-linking. The degree of cross-linking can alter the rate of loading a drug, and it can alter the rate of drug release. The present disclosure can encompass a hydrogel that consists of one of the following polymers or alternatively, that comprises one or more of the following polymers (e.g., as a block polymer). The polymers include, poly(ethylene oxide) (PEO), poly(propylene oxide) (PPO), poly(lactide-co-glycolic acid) (PLGA), poly(N-isopropylacrylamide) (PNIPAM), poly(propylene fumarate) (PPF), poly(caprolactone) (PCL), poly(urethane) (PU), and poly(organophosphazene) (POP). An example of a block polymer is PEO-PPO-PEO. In exclusionary embodiments, the present disclosure can exclude a hydrogel that includes PEO, PPO, PLGA, PNIPAM, PPF, PCL, PU or POP. The present disclosure also encompasses hydrogels that contain a cyclodextrin, where the cyclodextrin is cross-linked to hydrogel (see, Hoare et al (2008) Hydrogels in drug delivery: Progress and challenges. Polymer. 49:1993-2007). Hydrogels of the present disclosure can be ethylene vinylacetate, alginic acid, gums, polyvinylalcohol hydrogel; silicone hydrogel; polyvinylalcohol/dextran hydrogel; alginate hydrogel; alginate-pyrrole hydrogel; gelatin/chitosan hydrogel; polyacrylic acid hydrogel; photo crosslinked polyacrylic acid hydrogel; amidated pectin hydrogel; pectin hydrogel; gelatin hydrogel; polyethylene glycol (PEG) hydrogel; carboxymethylcellulose/gelatin hydrogel; chitosan hydrogel, as well as mixtures thereof, or copolymers thereof, and the like. Hydrogel with crosslinks are available (Lee et al (2003) Eur. J. Pharm. Biopharm. 56:407-412).

Printing active ingredients and excipients on dried hydrogels.

Dried hydrogel can take the form of a “xerogel” or of a film. Xerogel can be made by freeze drying a hydrogel. Film can be made by evaporative drying or casting from organic solutions. Spotting device can be used to apply microdrops in predetermined locations of dried hydrogel or on a film (see, e.g., U.S. Pat. No. 6,642,054 of Schermer). Where dried hydrogel or film takes the form of a layer, microdrops can be applied to one side only or to both sides. Where more than one type of drug is to be applied and where at least two of the drugs are incompatible with each other, or where a drug an an excipient are to be applied, and where these are incompatible with each other, these can be applied at different locations on the dried hydrogel or on the film. Drop size of microdrops can be, e.g., 0.05 nanoliters (nL)-10,000 nL, 0.5 nL-200 nL, 10 nL-100 nL, and so on. Drug, active ingredient, and/or excipient is not incorporated into the dried hydrogel, but is instead printed on its surface or surfaces. Printing on dried hydrogel avoids problems arising from incompatibility of drug, active ingredient, and/or excipient with the hydrogel itself. See, US2008/0095848 of Stabenau, which is incorporated by reference in its entirety.

Matrices (Chemical Composition and Methods for Manufacturing).

A matrix, carrier, or binder, can include, e.g., hydrogel, polyethylene oxide, polyvinylpyrrolidone, hydroxypropyl cellulose, ethyl cellulose, methylcellulose, alkyl cellulose, veegums clays, alginates, PVP, alginic acid, carboxymethylcellulose calcium, microcrystalline cellulose, polacrilin potassium, sodium alginate, corn starch, potato starch, pregelatinized starch, corn starch, modified starch, caranuba wax, montmorillonite clays such as bentonite, gums, shellac, agar, locust bean gum, gum karaya, pectin, tragacanth, and the like. In exclusionary embodiments, what can be excluded is one or more of the above polymers, clays, waxes, hydrogels, starches, and gums. A polyol can be used, for example, as a carrier. Polyols include propylene glycol and glycerol and the preferred (poly) alkoxy derivatives include poly alkoxy alcohols, in particular 2-(2-ethoxyethoxy) ethanol (Transcutol®).

The matrix can be manufactured by melt-granulation, melt-extrusion, using particulates, granules, bilayers, plasticizers, and the like (see, US2016/0151502 of Wright). Patch can be made with silicone adhesives disposed on a substrate, copolymers, block polymers, tackifying resins, hot melt coating processes (see, US2014/0349108 of Fung). Patch can be made with backings, release liner, pressure sensitive adhesives, silicone gel adhesives (see, US2014/0287642 of Kumar). Dermal patches can be made with excipient, disintegrant, swelling agent, films, binders, and the like (US2014/0079740 of Salama). Each of these patent documents is incorporated herein by reference in its entirety. Dermal patches, and methods of manufacture are detailed (Crowley et al (2007) Drug Development Industrial Pharmacy. 33:909-926; Repka et al (2007) Drug Development Industrial Pharmacy. 33:1043-1057).

Apertures and Pores

The present disclosure can encompass films, sheets, layers, membranes, and the like, including those with a plurality of apertures or pores. In some aspects, the apertures or pores have an average diameter of 20 nm, 40 nm, 50 nm, 100 nm, 200 nm, 300 nm, 400 nm, 500 nm, 600 nm, 800 nm, 0.001 mm, 0.002, 0.005 mm, 0.010 mm, 0.015 mm, 0.020 mm, 0.025 mm, 0.030 mm, 0.040 mm, 0.050 mm, 0.075 mm, 0.10 mm, 0.20 mm, 0.30 mm, 0.40 mm, 0.50 mm, and the like. Also, the pores can have a diameter range where the range is bracketed by any two of these values. In other aspects, the apertures or pores have a diameter in the range of 20-40 nm, 40-60 nm, 60-80 nm, 50-100 nm, 100-200 nm, 200-400 nm, 400-600 nm, 600-800 nm, 800-1,000 nm, 0.001-0.002 mm, 0.001-0.005 mm, 0.005-0.010 mm, 0.010-0.020 mm, 0.020-0.040 mm, 0.025-0.050 mm, 0.050-0.075 mm, 0.075-0.10 mm, 0.10-0.20 mm, 0.20 mm-0.40 mm, 0.25-0.50 mm, 0.50-0.75 mm, 0.50-1.00 mm, 0.1-0.2 mm, and so on. In exclusionary embodiments, the present disclosure can exclude films, sheets, layers, and the like, that have apertures or pores having any of the above average values, or that are describable by any of the above ranges.

Porous membranes can take the form of hydrophilic porous membranes and hydrophobic porous membranes, without implying any limitation. Hydrophobic membranes, such as hydrophobic polyethylene (PE) membranes, can be made more hydrophilic by alcohol or surfactants (see, WO2010/072233 of Calis). Pores in membranes of the present disclosure can have an average diameter of about 5 micrometers, about 10, about 15, about 20, about 25, about 30, about 40, about 50, about 60, about 70, about 80, about 90, about 100, about 110, about 120, about 130, about 140, about 150, about 160, about 170, about 180, about 190, or about 200 micrometers, and the like. Also, pores in the membranes can have an average diameter somewhere in the range 5-20 micrometers, 20-40 micrometers, 40-60 micrometers, 60-80 micrometers, 80-100 micrometers, 100-120 micrometers, 120-140 micrometers, 140-160 micrometers, 160-180 micrometers, 180-200 micrometers, and so on. In exclusionary embodiments, the present disclosure can exclude any membrane that is characterized by one of the above “about” values or that is characterizable by one of the above ranges.

For any given film, sheet, or layer, and the like, the area of a plurality of apertures or the area of a plurality of pores can occupy about 1%, about 2%, about 4%, about 6%, about 8%, about 10%, about 15%, about 20%, about 25%, about 30%, about 35%, about 40%, about 45%, about 50%, about 55%, about 60%, about 65%, about 70%, about 75%, about 80%, about 85%, about 90%, and the like of the surface area. In exclusionary embodiments, the present disclosure can exclude any film, sheet, or layer, where the area does not occupy one or more of the given percentage values, or where the area does not occupy a range between any two of the above given percentage values. The above parameters also can apply to a film, sheet, or layer, with perforations, where the value of the area for the perforation is measured flush with a surface of the film, sheet, or layer.

Solubilizers and Surfactants

Solubilizers such as detergents, surfactants, organic solvents, and chaotropic agents, are available for the present disclosure. These can be one or more of, polyethylene glycol (PEG), propylene glycol, dibutyl subacetate, glycerol, diethyl phthalate (phthalate esters), triacetin, citrate esters-triethyl citrate, acetyl triethyl citrate, tributyl citrate, acetyl tributyl citrate, benzyl benzoate, sorbitol, xylitol, bis(2-ethylhexyl) adipate, mineral oil, polyhydric alcohols such as glycerin and sorbitol, glycerol esters such as glycerol, triacetate; fatty acid triglycerides, polyoxyethylene sorbitan, fatty acid esters such as TWEENS, polyoxyethylene monoalkyl ethers such as BRIJ series and MYRJ series, sucrose monoesters, lanolin esters, lanolin ethers. These are available from Sigma-Aldrich, St. Louis, MO. In exclusionary embodiments, what can be excluded is any composition, formulation, dermal patch, and methods that comprise one or more of these solubilizers or surfactants.

The present disclosure can encompass compositions, formulations, devices, and methods, that comprise one or more surfactants, such as, sorbitan trioleate, sorbitan mono-oleate, sorbitan monolaurate, polyoxyethylene (20) sorbitan monolaurate, polyoxyethylene (20) sorbitan monooleate, oleyl polyoxyethylene (2) ether, stearyl polyoxyethylene (2) ether, lauryl polyoxyethylene (4) ether, block copolymers of oxyethylene and oxypropylene, diethylene glycol dioleate, tetrahydrofurfuryl oleate, ethyl oleate, isopropyl myristate, isopropyl palmitate, glyceryl monooleate, glyceryl monostearate, glyceryl monoricinoleate, cetyl alcohol, stearyl alcohol, cetyl pyridinium chloride, olive oil, glyceryl monolaurate, corn oil, cotton seed oil, and sunflower seed oil. In exclusionary embodiments, the present disclosure can exclude one or more of the above chemicals, and can also exclude a composition, formulations, device, and method that comprises any of the above chemicals.

Buffers and pH Values

The present disclosure can include formulations that contain a buffer with a pKa, as measured at room temperature, such as boric acid (pKa 9.2), CHES (pKa 9.5), bicine (pKa 8.3), HEPES (pKa 7.5), MES (pKa 6.1), MOPS (pKa 7.2), PIPES (pKa 6.8), Tris (pKa 8.1), imidazole (pKa 6.9), glycine (pKa 2.3), acetate (pKa 4.7), citrate (pKa 6.4), phosphate (pKa 7.21, 2.16, 12.32), malate (pKa 5.13), cacodylate (pKa 6.27), and the like. Also, the present disclosure can exclude formulations that include one or more of the above buffers, and can exclude a device that comprises one of these formulations. Without regard to any buffer, the present disclosure provides a formulation, or provides a component of a formulation, that has a pH value, as measurable at room temperature, of about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, and the like. In exclusionary embodiments, the present disclosure can exclude a formulation, or can exclude a component of a formulation, that has a pH value, as measurable at room temperature, of about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, and the like. The pH of component can be measured as a pure component, that is, prior to combining with other components to generate formulation.

Methods for Manufacturing Dermal Patches.

Administration of low water-solubility pharmaceuticals can be enhanced by formulating pharmaceutical in combination with a surfactant, or as a complex with hydrophilic cyclodextrins, or by using a nanosuspension (particle diameter in the nanomolar range, such as 50 nm to 150 nm) (see, Rao et al (2011) Int. J. Nanomedicine. 6:1245-1251). Nanoparticles can be made by milling, homogenization, or ultrasonication.

This describes solvent casting and direct milling methods of manufacture. Dermal patch can consist of two laminates, with an aqueous solution of an adhesive polymer being cast on an impermeable backing sheet. One type of adhesive film can comprise an alcoholic solution of hydroxypropyl cellulose and organic acids. This adhesive film stays in place for at least 12 hours, even in the presence of fluids. Adhesive patches can be made by solvent casting or by direct milling. In solvent casting, all excipients and the drug are dispersed in an organic solvent and coated on a sheet of release liner. After solvent evaporates, a thin layer of protective material is laminated on the sheet of coated release liner to form a laminate. The laminate is then cut into patches (Koyi and Khan (2015) Buccal patches: A review. Int. J. Pharmaceutical Sciences Res. 4:83-89).

In direct milling, patches are created without using solvents. Drugs and excipients are mixed by direct milling or by kneading, usually without any liquids present. After milling, the material is rolled on a release liner. A backing layer is then applied. Direct milling avoids the problem of residual solvents (Koyi and Khan (2015) Buccal patches: A review. Int. J. Pharmaceutical Sciences Res. 4:83-89).

This concerns the solvent casting method and hot melt extrusion method. Without implying any limitation, film can be made by solvent casting method and by hot melt extrusion method. Solvent casting involves dissolving water-soluble polymers to form a viscous solution. Excipients are dissolved into solvent to give a clear viscous solution. Then, both solutions are mixed (solution of water-soluble polymers; excipient solution) and then cast as a film, and then allowed to dry. This concerns hot melt extrusion. The drug or combination of drugs is in a dry state, and it is filled in a hopper, mixed, heated, and then extruded in a molten state. The molten mass that is formed is used to cast a film (Madhavi et al (2013) Buccal film drug delivery system—an innovative and emerging technology. J. Mol. Pharm. Org. Processing Res. Vol. 1, Issue 3 (6 pages)).

Without implying any limitation, mucoadhesive patches can be made by dissolving polymers in a solvent to produce a viscous solution. The polymers can be hydroxypropylmethyl cellulose (HPMC) E5LV and Carbopol® 940P. Polyethylene glycol 1000 can be included as a plasticizer. The solvent can be ethanol:chloroform (50:50). After creating the viscous solution, drugs can be dispersed in it. Then, the solution can be poured into molds for casting and dried for 24 hours. After drying, patches can be cut, for example, at 2 cm×2 cm. Each of the patches can contain, for example, 2 mg drug, 20 mg HPMC, 0.4 mg Carbopol, and 17 mg PEG100 (wt/vol) (see, Priya et al (2011) J. Pharm. Res. 3:56-65).

Suppliers of Chemical Reagents and Lab Equipment

Diclofenac, diclofenac potassium, diclofenac sodium, diclofenac diethylamine, capsaicin, eugenol, methyl-eugenol, o-eugenol, cannabidiolic acid, cannabidiol, delta-9-tetrahydrocannabinol (THC), polyisobutylene (PIB), dihydromyricetin, isopropyl myristate, various terpenes including limonene, are available from Sigma Aldrich, St. Louis, MO. Various molecular weights of polyisobutylene are available from BASF, Ludwigshafen, Germany. A composition that is “labeled” is detectable, either directly or indirectly, by spectroscopic, photochemical, biochemical, immunochemical, isotopic, or chemical methods. For example, useful labels include P-32, P-33, S-35, C-14, H-3 (tritium), 1-125, stable isotopes, epitope tags fluorescent dyes, electron-dense reagents, substrates, or enzymes, e.g., as used in enzyme-linked immunoassays, and fluorettes (see, e.g., Rozinov and Nolan (1998) Chem. Biol. 5:713-728).

Laboratory equipment is available from Thermo Fisher Scientific in Hayward, CA, VWR in Batavia, IL, and VWR in Radnor, PA, and Becton Dickonson in San Jose, CA. Vacuum mixers are available, and these include SpeedMixer from Synergy Devices, Ltd, Buckinghamshire, UK, Compact Vacuum Mixer from MTI Corporation, Richmond, CA, and dental laboratory mixtures made by Nuova, by Silfradent, by Renfert, by Mestra, and by Hagerwerken.

Franz Diffusion Cell for Assessing Rate of Drug Release

Franz diffusion cell is used to measure drug release kinetics from monolithic patches and from reservoir patches. Franz diffusion cells are described (see, Cavallari et al (2013) Eur. J. Pharm. Biopharm. 83:405-414; Franz (1968) On the diffusion of tritiated water through skin. J. Invest. Dermatol. 50:260; Balazs, Sipos, Danciu (2015) Biomedical Optics Express. 7:67-78; Simon et al (2016) Int. J. Pharmaceutics. 512:234-241; Jung et al (2016) Int. J. Cosmet. Sci. 38:646-650; Technical Brief 2009, Vol. 10, Development and Validation In Vitro Release Testing Methods for Semisolid Formulations, Particle Sciences, Bethlehem, PA). Franz diffusion cells and equipment for transdermal diffusion testing are available (Permegear, Hellertown, PA, and Teledyne Hanson Research, Chatsworth, LA).

The inventor used a Franz cell for assessing release kinetics, as described below. For testing release kinetics from transdermal monolithic patches, where a semi-solid adhesive matrix is used, the Franz cell has the following components, from top to bottom: (1) Stopper used to seal top of donor compartment; (2) Donor compartment; (3) Patch situated at very bottom of donor compartment, with adhesive side of patch attached to human cadaver skin; (4) Human cadaver skin located immediately under the patch; (5) Receiving compartment, located immediately below the skin. Receiving compartment is filled with ethanol/water solution; (6) Magnetic stirrer located at bottom of receiving compartment. The above descriptions refer to the inventor's lab work prior to initiating work on the menstrual pain patch.

For testing release kinetics from transdermal reservoir patches, the Franz cell has the following components, from top to bottom: (1) Stopper used to seal top of donor compartment; (2) Donor compartment; (3) Cream or gel in the donor compartment; (4) Microporous membrane (Solupor® from Lydall Performance Materials, Inc., Rochester, NH); (5) Human cadaver skin located under the microporous membrane; (6) Receiving compartment is filled with phosphate buffered saline at pH 6; (7) Magnetic stirrer at bottom of receiving compartment.

Receiving Solutions for the Franz Cell Used to Test Patches of the Present Disclosure.

This concerns receiving solutions that were used for testing the monolithic patches and dermal patches of the present disclosure. The receiving solution can be a saline solution for drugs that are soluble in water or alcohol-water solutions for drugs that are not well soluble in saline.

According to Bartosova and Bajgar (2012) Current Medicinal Chemistry. 19:4671-4677, dermal absorption involves these steps: (1) Penetration. Entry of a substance into a particular layer of the skin, such as the layer that is “stratum corneum;” (2) Permeation. This is penetration through one layer into another slayer, where the layers differ both structurally and functionally from each other; (3) Absorption. Uptake of substance into the lymphatics or into the bloodstream. The stratum corneum is lipophilic while, in contrast, the epidermis and dermis are hydrophilic. Thus, lipophilic molecules may pass at a greater rate through stratum corneum while, in contrast, hydrophilic molecules may pass at a greater rate through epidermis and dermis. Rate of transfer can be expressed by Frick's law: Jₛₛ=(Kₚ)(Cₒ). Jₛₛ is steady state flux per unit area. Kₚ is the permeability coefficient for a given solute in a given vehicle (centimeters per hour). Cₒ is the concentration of solute in the donor compartment. Kₚ predicts the penetration rate of a chemical at a given concentration from the same vehicle. Kₚ is independent of concentration and time.

According to Bartosova and Bajgar, supra, guidance for in vitro skin absorption tests is available from OECD (2004) OECD Guideline for the Testing of Chemicals. Skin Absorption: in vitro method. Pages 1-8). Diffusion dells are commonly used to measure in vitro skin absorption, and these can be of the static type or flow-through type. The Franz diffusion cell has the following structures, in order from top to bottom: Donor compartment (containing test substance, such as a drug); Membrane (supporting membrane where skin is positioned); Receptor compartment with sampling port that allows access to receptor compartment; Surrounding lower half of receptor compartment is water jacket for maintaining temperature; At bottom of interior of receptor compartment, and in contact with fluid in receptor compartment, is magnetic stirrer. Optionally, the researcher can include dermal absorption tests with standards, such as benzoic acid, caffeine, and testosterone.

Also, according to Bartosova and Bajgar, supra, dose concentrations up to 10 mg/cm2 or up to 10 microliters/cm2 are used. The skin sample is equilibrated with receptor fluid for 10-30 minutes before applying dose to skin. Barrier integrity of skin is checked by methods that determine transepidermal water loss or transcutaneous electrical resistance. Kinetic parameters that can be determined include flux (J), permeability coefficient (Kₚ), and diffusion coefficient (D). When the testing period comes to its scheduled end, for example, after three hours or after 24 hours, some of the test substance may still be located inside the skin, that is, in the membranes and cytosol of skin cells. Test substance inside the skin may optionally be included in the value for total substance that is absorbed.

Source of Human Skin

The inventor received dermatomed human cadaver skin from a tissue bank such as Science Care in Phoenix, AZ. The donor human skin was dermatomed in Science Care to thickness of about 250 micrometers consisting of stratum corneum and part of epidermal layer and shipped to the inventor on dry ice.

After receiving the donor human skin, the inventor prepared the skin for testing in a Franz cell by thawing the skin to room temperature, washing in distilled water and cutting in round pieces to fit the diameter of the Franz cell opening. Before placing the patch on the human skin, the inventor dried the skin with a paper tissue. The skin with the attached patch was placed between the Upper Donor Chamber and the Lower Receiving Chamber and clamped tightly. After that, the receiving solution was filled into the Receiving Chamber making sure no bubbles are trapped beneath the skin. The receptor solution was kept at 32° C., and stirred at about 150 rpm. The test lasts typically 24 hrs and aliquots of 150 L were withdrawn from the receiving chamber after different periods of time and analyzed via HPLC. Withdrawn amounts were replaced with an equal amount of receptor solution at 32° C. Display of the drug concentration in the receiving solution versus time was presented by graphs showing the kinetics of the transdermal passage of the drug from the patch through the skin into the receiving solution. The above descriptions refer to the inventor's lab work prior to initiating work on the menstrual pain patch.

Measuring Thickness of Films and of Patches.

Film thickness can be measured using a puncture test and texture analyzer, such as Instron® 3366-2716015, Germany (see, Priya et al (2011) J. Pharm. Res. 3:56-65).

Patch thickness can be measured with a screw gauge, where thickness can be measured at various different spots on the patch. To measure surface pH, patches can be allowed to swell for 2 hours on the surface of an agar plate (2% w/v), and the pH then measured with pH paper. Swelling can be measured by taking the weight each hour for six hours, after placing patch on an agar plate (see, Verma et al (2014) Effect of novel mucoadhesive patches of carvedilol on isoprenaline-induced tachycardia. J. Adv. Pharm. Technol. Res. 5:96-103). Residence time measured time that patch adheres to a mucosal membrane, where patch is glued to a substrate, with repeated up-and-down movement of the substrate until the patch detaches (see, Ismail et al (2003) Design and characteristics of mucoadhesive buccal patches containing cetyl pyridinium chloride. Acta Pharm. 53:199-212.

Dimple-Style Reservoir Vs. Balloon-Style Reservoir for Reservoir Patch Device.

In a balloon embodiment, the present disclosure can include a reservoir that is conformed like a sealed bag (or like a continual bag) or like a sealed balloon. In this embodiment, the reservoir is made of a material that is separate from the backing and separate from the permeable layer. In this embodiment, the reservoir may or may not be attached to a backing or permeable layer by way of an adhesive or heat seal.

In a dimple embodiment, the reservoir has on a distal side a backing that has a dimple (or outpouching) where the dimple is conformed to hold drug, and where the reservoir has on the proximal side a permeable layer. In other words, what prevents drugs from spilling out of the outpouching is this permeable layer.

The backing and permeable layer are attached to each other, to prevent leaking of the drug. Attachment can be via an adhesive or heat-sealing. The present disclosure can exclude devices where this attachment is by adhesive, and can exclude devices where this attachment is by heat-sealing.

The present disclosure can exclude devices with balloon reservoirs. In other embodiments, the present disclosure can exclude devices with a dimple reservoir.

In the dimple embodiment, the permeable layer can comprise a plurality of slits, a plurality of tiny holes, or by being made of a porous layer. The present disclosure can exclude devices with dimple reservoirs.

Dimple reservoir devices can include (or exclude) a layer that resides in-between the drug and permeable layer. Also, dimple reservoir devices can include (or exclude) a layer that resides on the side of the permeable layer facing the skin, where this layer is in substantial contact with the permeable layer. This layer that is on the side of the permeable layer of skin can be distal to the adhesive layer and peelable backing layer.

The permeable layer can comprise permeable polypropylene film (US2006/0024520; US20016/115585), permeable polyethylene film (U.S. Pat. No. 4,793,003; WO2006/070672); permeable polyurethane film (U.S. Pat. No. 9,566,423).

Shapes of Reservoirs that Alter Delivery Rate Over the Course of Time.

A reservoir of the present disclosure can be manufactured in a predetermined shape, so that rate of release of an active agent to the skin or to a mucosal surface varies over the course of hours, during the time frame when patch device is worn by a patient. For example, a reservoir can be conical, where the wide surface (base of cone) is situated at the distal portion of patch device and where the point of the cone is situated at the proximal portion of patch device. Proximal means the side of patch device closest to the skin, distal means the side of patch device farthest away from the skin. With a cone reservoir, the rate of drug transfer from patch to skin or mucosal surface gradually decreases over time. The reservoir can be hemispherical, resembling a gum drop, with base of gum drop closest to the proximal side of the patch device, and rounded surface of gum drop closest to the distal side of patch device. A hemispherical reservoir gives an initial rapid rate of drug release followed by a rapid decrease in rate of drug release. The reservoir can also have edges that are perpendicular to the skin-facing portion of the patch, that is, perpendicular to the peelable release of the dermal patch (in the event that the patch has a peelable release). See, U.S. Pat. No. 6,207,181 of Herrman, which is incorporated herein by reference in its entirety. The present disclosure can exclude a device with conical reservoir, with hemispherical reservoir, and/or hemispherical reservoir.

The reservoir device of the present disclosure can have only one conical reservoir, only two conical reservoirs, only three conical reservoirs, at least one conical reservoir, at least two conical reservoirs, at least three conical reservoirs. The reservoir device can have only one, only two, only three, at least one, at least two, at least three hemispherical reservoirs. The reservoir device can have only one, only two, only three, at least one, at least two, at least three perpendicular sided reservoirs. Moreover, the reservoir device can have only conical reservoir(s), only hemispherical reservoir(s), only parallel sided reservoir(s), a combination of only conical reservoir(s) and hemispherical reservoir(s), a combination of only conical reservoir(s) and parallel side reservoir(s), a combination of only hemispherical and parallel side reservoir(s), or a combination of all three of conical, parallel side, and hemispherical reservoirs. The present disclosure also provides reservoirs of an ambiguous shape, such as that resembling a wrinkled, partially filled balloon, either alone or in combination with a conical, hemispherical, and/or parallel shaped reservoir. The present disclosure also provides reservoirs having the shape of a hot dog, either alone or in combination with a conical, hemispherical, and/or parallel shaped reservoir.

Emulsions and Self-Emulsifying Agents

The present disclosure provides emulsions, emulsifying agents, self-emulsifying agents, creams, and lotions. The following provides examples of self-emulsifying agents. Self emulsifying drug delivery systems (SEDDS) and self-nano-emulsifying drug delivery systems (SNEDDS) have been reviewed (see, Cheriakov et al (2015) Expert Opin. Drug Deliv. 12:1121-1133). Self-emulsifying agents include glycerol monostearate, glycerol monooleate, and Cremophor RH40®. Cremophor RH40® is polyoxyl 40 hydrogenated castor oil. Cremophor EL® is polyoxyl 35 castor oil. These chemicals can be obtained from BASF Aktiengesellschaft, Ludwigshafen, Germany. In one aspect, the present disclosure can include formulations that comprise a self-emulsifying agent. In another aspect, the present disclosure can exclude formulations, and can exclude devices that comprise a self-emulsifying agent.

Solubilizer SL-11 is a self-emulsifying agent that provides a nanoemulsion suitable for containing a hydrophobic drug (NOF America Corp., Irvine, CA). Emulsion with particle size under 50 nanometers can be made by these steps: (1) Dissolve drug in a suitable solvent, such as ethanol; (2) Add the drug solution prepared in (1) to Solubilizer SL-11, thoroughly mix to completely dissolve the contents; (3) The drug/SL-11 solution with solvent is made; (4) Evaporate the solvent at 50 degrees for about 1 hour to remove the solvent, or remove the solvent under a nitrogen stream; (5) Concentrated solution of SL-11 and the drug is made; (6) Soft capsules can be prepared by using the concentrated solution in (5) (NOF America Corp., Irvine, CA).

The following provides another non-limiting example. According to Shah et al (1994) Int. J. Pharmaceutics. 106:15-23, self-emulsifying agents can be made with polyglycolyzed glycerides (PGG) with varying fatty acid and polyethylene glycol (PEG) chain lengths, where these produce the self-emulsification of oil in water. The quality of the resulting emulsions depends on the oil and emulsifier pair selected and on the concentration of PGG as the emulsifier. One suitable oil is an oil with medium-chain triglycerides (caprylic acid and capric acid; Neobee M5®). Another suitable oil is peanut oil. With formation of the emulsion, parameters that can be measured include droplet size distribution, droplet polarity, the release rate of the drug and the oil/water partition coefficient of the drug. PGG was found to be a workable emulsifier for use in self-emulsifying drug delivery systems (SEDDS) (Shah et al (1994) Int. J. Pharmaceutics. 106:15-23).

Yet another non-limiting example of a self-emulsifying agent is provided by Chambin et al (2004) Int. J. Pharmaceutics. 278:79-89. This describes a self-emulsifying system using Gelucire® 44/14, an excipient from the lauroyl macrogolglycerides family. The laboratory method involves producing a fine oil-in-water emulsion when introduced into an aqueous phase under gentle agitation as SEDDS. The advantage is improved solubility and bioavailability of poorly water-soluble drugs. Gelucire® 44/14 was ground into a powder by cryogenic grinding to produce solid oral dosage forms and resulting in formulations made of Gelucire® 44/14 and ketoprofen (90/10). Cryogenic grinding produced Gelucire® 44/14 in a powder form, where this process did not change its physical properties, emulsification capacities and dissolution performances of the formulation tested.

Devani (2004) J. Pharmacy Pharmacology. 56:307-316, provide the following example, using the drugs danazol and mefenamic acid. In self-emulsifying drug delivery systems (SEDDS), drugs are dispersed in an oil-surfactant mix that emulsifies on contact with water. Self-emulsifying systems can be based on the Labrafil family of polyglycolide oils, using Tween 80 and Tween 20 as surfactants. The more hydrophilic oil-surfactant mixes showed a greater ease of emulsification and a lower particle size. A linear relationship was observed between the hydrophile-lipophile balance (HLB) of the mix and the solubility of both danazol and mefenamic acid, with more hydrophilic mixes showing greater drug solubility values.

This provides another non-limiting example. Zupancic et al (2016) Eur. J. Pharm. Biopharm. 109:113-121 described emulsifying properties of SEDDS composed of long chain lipids (LC-SEDDS), medium chain lipids (MC-SEDDS), short chain lipids (SC-SEDDS) and no lipids (NL-SEDDS). The drug, enoxaparin, was incorporated via hydrophobic ion pairing in the chosen SEDDS. The average droplet size of chosen LC-SEDDS, MC-SEDDS and NL-SEDDS ranged between 30 and 40 nm. MC-SEEDS containing 30% Captex 8000, 30% Capmul MCM, 30% Cremophor EL and 10% propylene glycol and NL-SEDDS containing 31.5% Labrafil 1944, 22.5% Capmul PG-8, 9% propylene glycol, 27% Cremophor EL and 10% DMSO exhibited 2-fold higher mucus diffusion than LC-SEDDS. Both MC-SEDDS and NL-SEDDS showed sustained in vitro enoxaparin release. Orally administered MC-SEDDS and NL-SEDDS yielded an absolute enoxaparin bioavailability of 2.02% and 2.25%, respectively.

Further regarding emulsions, emulsifying agents can be characterized by Hydrophilic Lipophilic Balance (HLB). The HLB system is numbered 1 to 20. HLB values of 3 to 6 are lipophilic and these form water-in-oil emulsions (see, Vadlamudi, Hyndavi, and Tejeswari (2014) Current Drug Discovery Technologies. 11:169-180). HLB values of 8 to 18 are hydrophilic and these form oil-in-water emulsions (see, Grimberg, Nagel, and Aitken (1995) Environ. Sci. Technol. 29:1480-1487).

Permeation Enhancers

The present disclosure provides permeation enhancers, for example, for use with a dermal patch. Suitable permeation enhancers include, 2-(2-Ethoxyethoxy)ethanol (Transcutol®), 23-lauryl ether, Aprotinin, Azone (1-dodecyl-hexahydro-2H-azepin-2-one). Azone has also been described as being, “1-dodecyl azepan-2-one.”

Benzalkonium chloride, Cetylpyridinium chloride, Cetyltrimethylammonium bromide, Cyclodextrin, Dextran sulfate, Lauric acid, Lauric acid/propylene glycol, Lysophosphatidylcholine, Menthol, Methoxysalicylate, Methyl oleate, Oleic acid, Phosphatidylcholine, Polyoxyethylene, Polysorbate 80, Sodium EDTA, Sodium glycocholate, Sodium glycodeoxycholate, Sodium lauryl sulfate, Sodium salicylate, Sodium taurocholate, Sodium taurodeoxycholate, Sulfoxides, and Alkyl glycosides (see, Shojaei et al (June 2001) Systemic drug delivery via the buccal mucosal route. Pharmaceutical Technology. Pages 70-81). Other enhancers of the present disclosure are 1-octanol, 2-ethylhexanol, 1-nonanol, 1-decanol, and so on.

Permeation enhancers of the present disclosure can be a biphasic composition having a lipid phase and a water phase. Lipid phase can be prepared by mixing isopropyl palmitate and lecithin. Water phase can be a mixture of water and a surfactant. Surfactants can be Pluronic®, Pemulen®, Noveon®, or Carbopol®. Pemulen polymeric emulsifiers are high molecular weight, copolymers of acrylic acid and C10-C30 alkyl acrylate crosslinked with allyl pentaerythritol (Lubrizol, Inc. product sheet). Carbopol homopolymers are acrylic acid crosslinked with allyl sucrose or allyl pentaerythritol. Carbopol copolymers are acrylic acid and C10-C30 alkyl acrylate crosslinked with allyl pentaerythritol (Lubrizol, Inc. product sheet). Noveon® Polycarbophil, USP is a high molecular weight acrylic acid polymer crosslinked with divinyl glycol (Lubrizol, Inc. product sheet). Pluronic® polymers are block copolymers based on ethylene oxide and propylene oxide. They can function as antifoaming agents, wetting agents, dispersants, thickeners, and emulsifiers (BASF, Inc. product sheet). The present disclosure can exclude any formulation, composition, device, method, and such, that comprise one or more the molecules found in Pluronic®, Pemulen®, Noveon®, and Carbopol®.

PLOGel is “Pluronic Lecithin Organogel” (Pharmedica Enterprise, Selangor, Malaysia). PLOGel takes the form of an aqueous phase (240 mL poloxamer 407, potassium sorbate, water) and organic phase (60 mL lecithin, isopropyl palmitate, sorbic acid). The present disclosure can exclude any formulation, composition, device, method, and such, that comprise one or more of PLOGel, poloxamer 407, potassium sorbate, isopropyl palmitate, sorbic acid, lecithin, and the like.

In exclusionary embodiments, the present disclosure can exclude any formulation, composition, device, method, and such, that encompasses one of the above polymers, polymer compounds, and crosslinked polymer compositions.

In other exclusionary embodiments, the present disclosure can exclude compositions, formulations, dermal patches, layers, and the like, as well as methods, that comprise sulfoxides such as DMSO, Azones and Azone analogs such as laurocapram, transkarbams, 6-aminohexane acid esters, and can also exclude pyrrolidones such as 2-pyrrolidone, alcohols such as ethanol or decanol, glycols such as propylene glycol, surfactants, or vesicular carriers such as liposomes (see, Bartosova and Bajgar (2012) Curr. Med. Chem. 19:4671-4677).

Bioadhesive Materials

Bioadhesive polymer of the present disclosure, when swollen, creates a flexible network through which the drug can diffuse. The bioadhesive material serves as a matrix for retaining pharmaceutical agents, until patch is applied to the skin or to a mucosal surface of the consumer. Bioadhesive materials include, hydroxypropyl cellulose, carbopol, poly(vinyl pyrrolidone), sodium carboxymethyl cellulose, hydroxyethyl cellulose, polycarbophil, pectin, chitosan, xanthan gum, locust bean gum, hydroxypropyl methylcellulose, poly(vinyl alcohol), poly(isoprene), poly(isobutylene) (see, Shojaei et al (June 2001) Systemic drug delivery via the buccal mucosal route. Pharmaceutical Technology. Pages 70-81).

Dermal Patches with a Plurality of Adhesive Layers.

The present disclosure provides dermal patches, laminated sheets, and related methods that comprise a plurality of adhesive layers. In one embodiment, a monolithic patch has these layers, from most distal to most proximal: (1) Backing; (2) Adhesive; (3) Carrier layer containing active agent; (4) Contact adhesive, and (5) Protective liner. In an exclusionary embodiment, the present disclosure can exclude this embodiment.

In another embodiment that is characterized by having a “rate controlling layer,” the monolithic patch has these layers, from distal to proximal: (1) Backing; (2) Adhesive; (3) Carrier layer containing active agent; (4) Adhesive layer; (5) Rate controlling polymer layer; (6) Adhesive layer; and (7) Protective liner. In an exclusionary embodiment, the present disclosure can exclude this embodiment.

The following concerns an embodiment where there is a “carrier layer” and where carrier layer is surrounded by and in contact with, on distal surface and on lateral surfaces, an adhesive layer, and where carrier layer is surrounded by and in contact with, on proximal surface, an “active ingredient permeable skin contact adhesive layer.” More generally, the present disclosure encompasses a “hat embodiment” taking the form of a dermal patch or other medical device where a first layer has a distal surface, proximal surface, and lateral surfaces. In this “hat embodiment” the distal surface, proximal surface, and lateral surfaces, are all surrounded by and in contact with a “hat layer.” The hat layer can be an adhesive layer or it can be an impermeable backing layer. The term “hat embodiment” and “hat layer” refer to the fact that the “hat layer” covers the first layer, in the same way that a man's hat covers the top of his head as well as his ears, forehead, and back of his head. The present disclosure provides a device with these layers, from distal to proximal: (1) Backing; (2) Adhesive; (3) Carrier layer; (4) Active ingredient permeable skin contact adhesive; and (5) Protective liner. In this embodiment, the “hat” can cover the lateral sides of the carrier layer and also cover the lateral sides of the “active ingredient permeable skin contact adhesive layer.” In an exclusionary embodiment, the present disclosure can exclude the above “hat” embodiment.

In another “hat” embodiment, the present disclosure provides, from distal to proximal: (1) Backing; (2) Adhesive layer; (3) Carrier layer; (4) Active ingredient permeable layer; (5) Rate-controlling polymer layer; and (6) Active ingredient permeable skin contact layer. The “hat” takes the form of a backing plus an adhesive layer, and the hat covers the lateral sides of all four of these layers: carrier layer, active ingredient permeable layer, rate-controlling polymer layer, and active ingredient permeable skin contact layer. In an exclusionary embodiment, the present disclosure can exclude the above “hat” embodiment.

In other exclusionary embodiments, the present disclosure can exclude devices where: (1) Carrier layer is in direct and substantial contact with backing layer; (2) Carrier layer is in direct and substantial contact with an adhesive layer; (3) Adhesive layer is in direct and substantial contact with rate-controlling polymer layer; (4) An adhesive layer is in direct and substantial contact with protective liner; (5) Where the device comprises a “hat” configuration of layers; (6) Carrier layer is in direct and substantial contact with active ingredient permeable layer, (7) Active ingredient permeable skin contact layer is in direct and substantial contact with protective layer; (8) Active ingredient permeable skin contact layer is in direct and substantial contact with release liner or protective liner; (9) Where at least part of the device has a “hat” configuration and where only one layer is covered (surrounded on proximal face and on lateral faces) by the hat; (10) Where at least part of device has “hat” configuration and where only two layers are covered (surrounded on proximal face and on lateral faces) by the hat; (11) Where at least part of device has “hat” configuration and where only three layers are covered (surrounded on proximal face and on lateral faces) by the hat; (12) Where at least part of device has “hat” configuration and where only four layers are covered (surrounded on proximal face and on lateral faces) by the hat. The exclusionary embodiments of the present disclosure encompass any combination of the above exclusions. The above may apply to reservoir patches where, optionally, “reservoir” takes the place of “carrier layer.” Also, the above may apply to monolith patches.

Manufacturing Processes and Equipment

Sealing two strips together at the edges, and coordinating transverse sealing to create pouches and filling of the pouches.

What is provided is a method to feed two strips into a machine with rollers to move the strips at the same speed, and to cause the two strips to move downwards, where the first face of the first strip is caused to contact the first face of the second strip. The first face is caused to contact the first face of the second strip, in preparation for heating the edges of the two strips, thus sealing the two strips together, and in preparation for transverse heating, with heating at intervals of distance and time, thus creating a plurality of pockets in the sandwich of the two strips. When the two strips are moved downwards, the first strip and second strip are situated to form a thicker sandwich that moves downwards. Heaters resembling wheels or rollers, clamp down on the edges of the 2-strip sandwich, causing the 2-strip sandwich to form a long, closed tube. While the 2-strip sandwich moves downwards, what simultaneously occurs is simultaneous heating/sealing of a pair of transverse clamps. The transverse clamps create separate pouches in the long 2-strip sandwich. When the heated bars clamp down, what is created is a top seal of a previously-filled pouch, and the bottom seal of a pouch that has yet to be filled. Simultaneously occurring with heating/sealing at the edges by the heated wheels, and simultaneously occurring with heating by the transverse bars, is filling of each pouch as it is created, where filling is by a long tube that reaches down into the long sandwich to fill each pouch as it is created. See, U.S. Pat. No. 6,871,477 of Tucker, which is incorporated by reference in its entirety. The first strip can comprise an adhesive layer and permeable membrane, the second strip can be an impermeable backing, and the gel can comprise a cannabinoid in gel form.

Unrolling Three Different Layers from Rolls, Stripping Off Release Liners from Two of the Layers, Aligning the Three Layers Together to Form a Complex, and Rolling the Complex on to a Roll.

The present disclosure provides machinery that can unroll a plurality of rolls, optionally with stripping off a release-layer from one or more of the rolls, and taking up the stripped-off release-layer on an empty rotating drum or roll. For example, three different rolls can contain three different laminates, the first laminate comprising: (1) Protective backing; (2) Combined zone of transport enhancement and zone of containment; and (3) Release layer. The second laminate can comprise: (1) Adhesive layer; (2) Zone of transport control; and (3) Release liner. And the third laminate can comprise: (1) Support film; (2) Adhesive; and (3) Removable liner that is not removed during the above-mentioned method. Machinery can include three rolls on three rotating mechanisms of first, second, and third laminate, respectively. Machinery can include take-up rolls for taking up release liners. Machinery can include a pair of rollers situated on opposite sides of a moving sandwich of first laminate and second laminate for use in bringing the two laminates together. Also, machinery can include a pair of rollers situated on opposite sides of the nascent 3-part sandwich, where the 3-part sandwich takes the form of the combined (in contact with each other) first and second laminate and the entering third laminate. The entering third laminate is simultaneously unrolled from its roll and then combined with the complex of the first and second laminate. The final product is then moved, by way of pairs of rollers situated on opposite sides of the moving final product, and also moved by way of individual rollers, e.g., rollers called over roller, under roll, and over idler roller. The above-disclosed machinery can also include a device for sealing laminates together, a corona discharge for enhancing the sealing of the laminates together, a device for depositing a drug or adhesive or other composition on one or more of the laminates as the laminate is unrolled from its roll, and cutting devices for separating the sandwich of three laminates into patches. See, U.S. Pat. No. 5,370,924, which is incorporated herein by reference in its entirety.

A layered device may be assembled and then sealed by vacuum forming or by heat sealing without vacuum. In exclusionary embodiment, the present disclosure can exclude machinery, methods, and patches, made using one or more of vacuum forming, heat sealing, corona discharge, one or more crimp rolls, or cooperating nip.

Providing a Platen with Bar-Like Regions Separated by Channels, and Using the Platen to Stamp a Laminate, and to Provide Pressure on Regions that Need to be Collapsed, while Refraining from Providing Pressure on Regions that Contain Drug and Matrix.

The present disclosure provides machinery, such as a plate with bar-like regions separated by channels, and where the bar-like regions are optionally heated. The platen can be used to selectively compress parts of a laminate, where the laminate (the “workpiece”) comprises an upper layer that is a cellular region and a lower layer that is a skin adhesive. The platen selectively compresses the distal sides (the right edge and left edge), resulting in collapse of the distal sides of the laminate. Optionally, only the part of the laminate destined to be collapsed is provided with the adhesive. The cellular region can be reticulated or it can be non-reticulated. The cellular region can be made of foamed thermoplastic resin. Cell size can be about 0.05, about 0.1, about 0.2, about 0.4, about 0.6, about 0.80, about 1.0, about 1.2, about 1.4, about 1.6, about 1.8, about 2.0, about 2.5, about 3.0, or about 4.0 millimeters. Collapsed regions are such that drugs cannot easily pass through collapsed regions. In embodiments, non-collapsed central area (the area that resided under the channel during platen-manufacturing process) can contain a distally-situated layer of drug-releasing matrix (which contains drug) in contact with a proximally-situated layer of a medium through which drug can diffuse. The layer of medium through which drug can diffuse can be, e.g., gel, cream, or ointment. “Distal” means away from the skin when patch is attached to skin, and “proximal” means on the side of patch that is closest to skin, when patch is attached to skin. The compressed lateral parts of the patch may be called “straps.” See, U.S. Pat. No. 5,505,958 of Bello, which is incorporated herein by reference in its entirety.

In exclusionary embodiments, the present disclosure can exclude patch devices with non-compressed cellular region, patch devices with compressed cellular region, layered structures with a distally-situated drug matrix and a proximally-situated gel, cream, ointment, or other medium through which drug can diffuse on its way to skin. Also, the present disclosure can exclude any composition, laminate, layered structure, and patch that was made via heating of a layered structure or via heating of a laminate.

Placing Drugs Between Two Webs, Sealing Two Webs Together, Crimping the Sealed Webs to Form Pockets, and Cutting the Sealed Web.

The present disclosure provides machinery and methods for using, as starting material, two different webs, each on a roller, where each web comprises one or more of a film, adhesive layers, impermeable layers, porous layers, and the like. The finished product takes the form of the two webs that are sealed together, and where an active ingredient, such as a composition comprising one or more of eugenol, capsaicin, cannabidiol (CBD), cannabidiolic acid (CBDA), diclofenac, and β-caryophyllene (BCAP), and one or more of a permeation enhancer, preservative, and antioxidant (such as ascorbyl palmitate), is contained therein. In the method, a first supply roll provides one web and a second supply roll provides a second web. Machinery at various “stations” modify one of the webs or modify both of the webs, as the webs move along a conveyor belt. One station, which is optional, is a corona discharge station. The corona discharge modifies the surface chemistry of one or both of the webs, prior to marriage of the two webs together by operation of two crimp rolls. The corona discharge modifies the surface chemistry to improve adhesive properties of the first web and/or of the second web. Corona discharge is preferred where dissimilar materials (one material of first web, and other material of second web) are to be adhesively joined. Dissimilar materials can be, e.g., polyester polymer and ethylene acrylic acid polymer.

Another station is the deposit station, which deposits active substance on one of the webs, as the web moves towards the crimp roll. A deposit station can include a reservoir that contains drug and tube leading from reservoir to location on the web surface where drug is to be deposited. The deposit station preferably occurs after the corona station. Also, the deposit station and corona station preferably act on the same web, though optionally deposit station can operate on the first web and corona station can operate on second web. The two webs are securely fastened together in a station taking the form of a first crimp roll and a second crimp roll. These rolls resemble gears, in that first crimp roll has projections and second crimp roll has depressions, which act meashingly in the manner of a “tongue-and-groove” to compress the two webs together and, at the same time, to stamp the joined webs into a pocket-like shape. The regions of the first crimp roll and second crimp roll that mesh together are called a “cooperating nip.”

Finally, after the webs pass through the corona station, drug deposit station, and crimp rolls, the joined webs are cut by rotary die cutter, to create flexible packages or flexible patches suitable for marketing. Motors can drive rollers. Also, motors can drive crimp rolls. See, U.S. Pat. No. 4,782,647 of Williams which is incorporated herein by reference.

Separating Cut Patches from a Strip of Non-Cut Patches, and Transferring Cut Patches to a Carrier.

This describes only one step in the procedure for making adhesive patches, e.g., monolithic devices and reservoir devices. The procedure involves a cutter, transfer devices resembling wedges, and rollers. The rollers function to move a first web and a second web, in the manner of a conveyor belt. The first web takes the form of an auxiliary layer film on top, and then just under it, a drug-containing adhesive layer that sits on top of a carrier film. The first web, which has these three layers, is then later on supplemented by a process layer, where the result is a web consisting of four layers (process layer on top, then auxiliary layer film, then drug-containing adhesive layer, and on the bottom, carrier film). An earlier-occurring cutting process has cut the auxiliary layer film and the drug-containing adhesive layer into blocks. The first web is moved in one direction, e.g., to the left, and then with the help of the transfer devices resembling wedges, the squares are separated from the carrier film (the carrier film is then moved away to the right) and also separated from the combination of auxiliary layer film and process layer (which is moved upwards), where the squares end up residing on a carrier film. At this point the blocks are separated from each other, and any scrap that had been created with the cutting process is then discarded. This refers to the situation where cutting creates discrete blocks and creates scraps in between the blocks. The supporting film supports the blocks and moves away to the left. See, U.S. Pat. No. 6,059,913 of Asmussen, which is incorporated herein in its entirety.

Cutting Laminate to Create a Fully Cut-Out Region and, within it, a Partially Cut (Scored) Region.

Machinery, methods, and workpiece of the present disclosure comprises sheet of laminate, where shapes of the sheet (rectangles, ovals, circles) are cut fully through the laminate, and where the edges of the cut-out laminate is called, “periphery” (outer cut). Where the cut-out laminate is circular, the periphery is the same as the circumferential region. In addition to being cut at the periphery, the sheet is simultaneously cut during the cutting operation in a region within the periphery (inner cut). The inner cut has a smaller diameter than the outer cut. Also, the inner cut is to a shorter depth than the outer cut. In the case of a 3-layer laminate (release layer; pressure-sensitive adhesive, backing), the outer cut slices through all three layers, but the inner cut slices only partially through the top layer (the release layer). This partial cutting is more properly called, “scoring” rather than “cutting.” The goal of this 2-distance cutting method is to score the release layer to facilitate easy removal of the liner by the user, and at the same time, to avoid leaking of adhesive from the patch during storage of the patch. Machinery for the 2-distance cutting method can take the form of a roller covered with cutting stampers (similar to cookie-cutters). Each cookie cutter stamps all the way through the laminate. Within each cookie cutter resides a second (smaller diameter) cookie cutter which is sized so that it only cuts partially through the top layer of the laminate (thus only scoring the top layer). In an alternative machinery, the first roller bears an array of only the larger diameter (and longer cutting distance) cookie cutters, while the second roller bears an array of the smaller diameter (and scoring distance) cookie cutters. In operation, the two rollers operate simultaneously, and the cookie cutters on the first roller are aligned exactly with the cookie cutters on the second roller and, in operation the cutting (cutting through all layers) occurs simultaneously with scoring, for each patch. See, U.S. Pat. No. 5,656,285 of Sablotsky, which is incorporated by reference in its entirety. In addition to the one cutting roller (or to the two cutting rollers), the machinery can have a pressure roller and a support roller, for use in driving the sheet of laminate. In exclusionary embodiments, the present disclosure can exclude an adhesive dermal patch that has a scored region, such as a scored release layer.

Efficient Separation of Punched Patches from Scrap Web.

During manufacture of adhesive patches, patches are stamped out from, or cut from, a sheet consisting of various layers. These layers may include backing, matrix containing a drug, skin adhesive, and release layer. During cutting, some of the punched patches that have not yet been separated from the scrap web may cling to the scrap web as the scrap web is pulled away from the sheet. Where this clinging is maintained as the scrap web is pulled away, the result will be undesirable discarding of the clinging punched patches along with the scrap web. This type of undesired clinging can be increased by flow of adhesive out of the edges of the punched patch, followed by flow of the adhesive to contact the scrap web. Efficient separation of punched patches can be accomplished by way of a probe or probes that contact the punched patch and shove the punched patch onto a horizontally moving conveyor belt as the scrap web is drawn upwards for eventual discard. The probe can take the form of a rotating roller where the roller is covered with blocks having the same shape and exactly the same dimensions (or dimensioned to be about 5% smaller, about 10% smaller, about 15% smaller, about 20 smaller, and the like, in area, as compared to the punched patch). The blocks can have a shape, as viewed from “above,” that is square, rectangular, oval, round, etc., and to have a shape corresponding to the punched patch. Thus, as the roller rotates, each block presses down on a corresponding punched patch (as the punched patch continues to move on the conveyor belt) while the scrap web is simultaneously detached and drawn upwards by the rotation of the roller. An alternative to using a roller covered with block probes, is a roller covered with flexible bristles. As the roller rotates, the bristles press springfully down on the punched patches, the bristles remaining bent, causing the punched patches to separate from the scrap web. At the same time, the bristles pressing on the scrap web are greatly bent at first, but as the scrap web is pulled upwards, the bristles spring out to their full (un-bent) length. See, US2017/0136648 of Grader, which is incorporated herein by reference in its entirety. In an exclusionary embodiment, the present disclosure can exclude manufacturing machinery and methods, comprising a roller with blocks or a roller with bristles, for use in preventing punched patches from adhering to the scrap web.

Making Powders and Gels.

The present disclosure provides a method for making powders, comprising combining and mixing an active ingredient such as eugenol, capsaicin, cannabidiol (CBD), cannabidiolic acid (CBDA), diclofenac, or β-caryophyllene (BCAP) with one or more of ethanol, PEG, isopropyl myristate, carbopol, triethanol, permeation enhancer, acetone, and water, and then drying using heat, then milling to form a powder, and then optionally adding a lower alcohol such as ethanol, propanol, or butanol, to form a gel.

Preventing Formation of Drug Crystals.

If undesired crystals form in any solution, such as solution for adhesive, solution for adhesive/drug mixture, or solution for drug matrix for placing in a reservoir, heating steps are available to prevent such crystals. The procedure for casting a film can involve use of a casting solution, a drying oven, a laminator, and an annealing oven. “Annealing” refers to heating followed by cooling to room temperature. What can get annealed is a liquid dispersion or an article formed from liquid dispersion. Undesired crystals can occur after die-cutting has occurred. Undesired crystals can occur even after removing water with desiccants. A method that avoids formation of drug crystals can involve casting the reservoir solution to form a drug reservoir film. Drug reservoir film can be on a backing of aluminized polyethylene terephthalate (Scotchpak®). After casting, the composition on the backing is put in an oven to remove solvent, e.g., chloroform, where the temperature is greater than melting point of the drug's crystals. This is followed by packaging, and further heating (see, e.g., U.S. Pat. No. 7,169,409 of Dohner, which is incorporated by reference in its entirety). First heating can be at, e.g., about 50, about 55, about 60, about 65, about 70, about 75, about 80, about 85, about 90, about 95 degrees C. Second heating can be at, e.g., about 50, about 55, about 60, about 65, about 70, about 75, about 80, about 85, about 90, about 95 degrees C. First heating step and second heating step can each be selected from one of 10 min, 15 min, 20 min, 30 min 40 min, 60 min, 4 hours, 8 hours, 12 hours, 16 hours, 20 hours, or 24 hours.

### Exclusionary Embodiments

The present disclosure can exclude a composition, formulation, dermal patch, methods of use, methods of manufacture, that comprise one or more of the following: eugenol, capsaicin, cannabidiol (CBD), cannabidiolic acid (CBDA), diclofenac, β-caryophyllene (BCAP), 2-arachidonoylglycerol, curcumin, glyceryl monooleate, glyceryl monostearate, lecithin, acacia gum, xylitol, carboxymethylcellulose, a self-emulsifying agents, glycerol monostearate, glycerol monooleate, Cremophor RH40®, Cremophor EL®, hydroxypropyl cellulose, carbopol, poly(vinyl pyrrolidone), sodium carboxymethyl cellulose, hydroxyethyl cellulose, polycarbophil, pectin, chitosan, xanthan gum, locust bean gum, hydroxypropyl methylcellulose, poly(vinyl alcohol), poly(isoprene), poly(isobutylene). The present disclosure can also exclude one or more of, 23-lauryl ether, Aprotinin, Azone, Benzalkonium chloride, Cetylpyridinium chloride, Cetyltrimethylammonium bromide, Cyclodextrin, Dextran sulfate, Lauric acid, Lauric acid/propylene glycol, Lysophosphatidylcholine, Menthol, Methoxysalicylate, Methyl oleate, Oleic acid, Phosphatidylcholine, Polyoxyethylene, Polysorbate 80, Sodium EDTA, Sodium glycocholate, Sodium glycodeoxycholate, Sodium lauryl sulfate, Sodium salicylate, Sodium taurocholate, Sodium taurodeoxycholate, Sulfoxides, and Alkyl glycosides. What can also be excluded is a formulation, composition, device, or method, that comprises pregelatinized starch, gelatinized starch, gelatinized corn starch, glycogelatin, alpha-tocopherol, hemp oil, THC, CBD, gum acacia, sorbitol, xylitol, soy lecithin, a complex of two different gels (one with net negative charge and the other with net positive charge), and a composition that comprise a solvent with a cosolvent.

What can be excluded is pharmaceutical compositions with 1-10% enhancer. What can be excluded is pharmaceutical compositions with 0.5-5% neutralizer, or with any amount of neutralizer What can be excluded is compositions with greater than 0%-5% by weight isopropyl myristate, or with any amount thereof. What can be excluded is pharmaceutical compositions with 0%-10% by weight carbopol, or with any amount of carbopol. What can be excluded is pharmaceutical compositions with about 10% ethanol, about 15%, about 20%, about 24%, about 30%, about 35%, about 40%, about 45%, about 50%, about 55%, about 60%, about 65%, about 70%, about 75%, about 80%, about 85%, about 90% ethanol.

For delivery of cannabinoids, for example, a system of solvent/cosolvent can be ethanol (solvent)/propylene glycol (cosolvent). Solvents can be anhydrous alcohol, ethanol, propanol, or isopropanol. Cosolvent can be propylene glycol or PEG. Ratio of solvent/cosolvent (by weight) can be about 5/95, about 10/90, about 15/85, about 20/80, about 25/75, about 30/70, about 35/65, about 40/60, about 45/55, about 50/50, about 55/45, about 60/40, about 65/35, about 70/30, about 75/25, about 80/20, about 85/15, about 90/10, about 95/5, and the like. In exclusionary embodiments, the present disclosure can exclude solvent/cosolvent compositions where the ratio is, 5/95, about 10/90, about 15/85, about 20/80, about 25/75, about 30/70, about 35/65, about 40/60, about 45/55, about 50/50, about 55/45, about 60/40, about 65/35, about 70/30, about 75/25, about 80/20, about 85/15, about 90/10, about 95/5, and the like.

Further Exclusionary Embodiments Relating to Ethanol, PEG, Phospholipids, Monoglycerides, Diglycerides, and Triglycerides.

What can be excluded is a formulation with an ethanol content, by weight, of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%. Also, what can be excluded is a formulation with an ethanol content, by weight, that encompasses about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%. Moreover, what can be excluded is a formulation with an ethanol content, by weight, that encompasses (range that equals or range that includes) a range that is 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, or 95-100%. What can also be excluded is a device that encompasses one or more of the above formulations.

The present disclosure can provide a formulation that comprises ethanol and propylene glycol (or glycerol monostearate, or glycerol monooleate, or monoglyceride, or diglyceride, or triglyceride, or PEG, or phospholipid, or surfactant), and where the ratio (weight/weight basis) is about 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, or 95/5. In exclusionary embodiments, what can also be excluded is a formulation that comprises ethanol and propylene glycol (or glycerol monostearate, or glycerol monooleate, or monoglyceride, or diglyceride, or triglyceride, or PEG, or phospholipid, or surfactant), and where the ratio (weight/weight basis) is about 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, or 95/5.

In embodiments, the present disclosure can exclude a reservoir-type device where backing does not directly contact reservoir; or where reservoir does not directly contact a hydrophilic porous membrane; or where hydrophilic porous membrane does not directly contact a release liner; or where reservoir does not contain all of: (1) a liquid carrier, (2) a gelling agent, and (3) one or more of eugenol, capsaicin, cannabidiol (CBD), cannabidiolic acid (CBDA), diclofenac, and β-caryophyllene (BCAP). Also, what can be excluded is a reservoir-type device that does not comprise all of the above.

In embodiments, what can be excluded is an adhesive polymer, or a device comprising an adhesive polymer, where the adhesive polymer reacts with amines. Also what can be excluded, is an adhesive polymer, or a device comprising an adhesive polymer, where the adhesive polymer has any free hydroxyl groups.

What can be excluded is an adhesive polymer, or a device comprising an adhesive polymer, where the adhesive polymer has over 1 free hydroxyl groups per 100 atoms of the adhesive polymer.

What can be excluded is an adhesive polymer, or a device comprising an adhesive polymer, where the adhesive polymer has over 5 free hydroxyl groups per 100 atoms of the adhesive polymer.

Also, what can be excluded is an adhesive polymer, or a device comprising an adhesive polymer, where the adhesive polymer has over 10 free hydroxyl groups per 100 atoms of the adhesive polymer.

Moreover, what can be excluded is an adhesive polymer, or a device comprising an adhesive polymer, where the adhesive polymer has over 20 free hydroxyl groups per 100 atoms of the adhesive polymer, and so on.

In embodiments, what can be excluded is a monolith-type device where a backing is not in direct contact with a matrix of skin adhesive; where matrix of skin adhesive is not in direct contact with a releasable liner; where matrix does not comprise one or more of eugenol, capsaicin, cannabidiol (CBD), cannabidiolic acid (CBDA), diclofenac, and β-caryophyllene (BCAP); or all of the above.

What can also be excluded is a preparation, or a device comprising a preparation, where the preparation has over 1% gelling agent, over 2%, over 3%, over 4%, over 5%, over 6%, over 7%, over 8%, over 9%, over 10%, over 12%, over 14%, or over 16%, of gelling agent. Also, what can be excluded is a preparation, or a device comprising a preparation, where the preparation has under 1% gelling agent, under 2%, under 3%, under 4%, under 5%, under 6%, under 7%, under 8%, under 9%, under 10%, under 12%, under 14%, or under 16%, of gelling agent.

What can also be excluded is a preparation, or a device comprising a preparation, where the preparation has over 1% penetration enhancer, over 2%, over 3%, over 4%, over 5%, over 6%, over 7%, over 8%, over 9%, over 10%, over 12%, over 14%, or over 16%, of penetration enhancer. Also, what can be excluded is a preparation, or a device comprising a preparation, where the preparation has under 1% penetration enhancer, under 2%, under 3%, under 4%, under 5%, under 6%, under 7%, under 8%, under 9%, under 10%, under 12%, under 14%, or under 16%, of penetration enhancer.

In other embodiments, what can be excluded is a preparation, a composition, a device comprising a preparation, a device comprising a composition, where said preparation or composition has a eugenol, capsaicin, cannabidiol (CBD), cannabidiolic acid (CBDA), diclofenac, or β-caryophyllene (BCAP) (or combined weight of eugenol, capsaicin, cannabidiol (CBD), cannabidiolic acid (CBDA), diclofenac, and β-caryophyllene (BCAP)) content by weight of under 1%, under 2%, under 3%, under 4%, under 5%, under 6%, under 8%, under 10%, under 12%, under 14%, under 16%, under 18%, under 20%, under 25%, under 30%, under 35%, under 40%, under 45%, under 50%, under 55%, under 60%, under 65%, under 70%, under 75%, and so on. Also, what can be excluded is a preparation, a composition, a device comprising a preparation, a device comprising a composition, where said preparation or composition has a eugenol, capsaicin, cannabidiol (CBD), cannabidiolic acid (CBDA), diclofenac, or β-caryophyllene (BCAP) (or combined weight of eugenol, capsaicin, cannabidiol (CBD), cannabidiolic acid (CBDA), diclofenac, and β-caryophyllene (BCAP)) content by weight that is greater than 5%, greater than 6%, greater than 7%, greater than 8%, greater than 10%, greater than 12%, greater than 14%, greater than 16%, greater than 18%, greater than 20%, greater than 25%, greater than 30%, greater than 35%, greater than 40%, greater than 45%, greater than 50%, greater than 55%, greater than 60%, greater than 65%, greater than 70%, and so on. In embodiments, what can be excluded is a preparation, a composition, a device comprising a preparation, or a device comprising a composition, where the percent by weight is defined by one or more of the above “under” or “greater than” parameters. “Composition” can refer to, for example, matrix of a skin adhesive, or to fluid in hydrophilic porous membrane, and so on. Alternatively, the present disclosure can comprise one or more of the above compositions, as set forth by “under” parameters or “greater than” parameters.

Moreover, in embodiments what can be excluded is any device that does not include an occlusive system polymer film, that does not include a polyethylene occlusive polymer film, that does not include a PET occlusive polymer film, that does not include an occlusive polymer film made of both polyethylene and PET. Also, what can be excluded is a device that has an overlay patch, and a device that does not comprise an overlay patch.

In embodiments, polar organic liquid can comprise, or can exclude, one or more of methanol, ethanol, propanol, isopropanol, butanol, pentanol, acetic acid, propionic acid, butyric acid, valeric acid, caproic acid, caprylic acid, capric acid, lauric acid, myristic acid palmitic acid, stearic acid, palmitoleic acid, oleic acid, linoleic acid, linolenic acid, linear alkanes of 5, 6, 7, 8, 9, 10, 11, 12, 13, 14, 15, 16, 17, 18, 19, 20, 21, 22, 23, 24, or more carbons, branched chain alkanes with a backbone of 5, 6, 7, 8, 9, 10, 11, 12, 13, 14, 15, 16, 17, 18, 19, 20, 21, 22, 23, 24, or more carbons, linear alkenes (olefins) of 5, 6, 7, 8, 9, 10, 11, 12, 13, 14, 15, 16, 17, 18, 19, 20, 21, 22, 23, 24, or more carbons, branched chain alkenes (olefins) with a backbone of 5, 6, 7, 8, 9, 10, 11, 12, 13, 14, 15, 16, 17, 18, 19, 20, 21, 22, 23, 24, or more carbons, and so on. Alternatively, the present disclosure can comprise one or more of the above polar organic liquids.

The present disclosure can exclude a composition, device, method, that comprises an essential oil, a plant oil, a vegetable oil, or a fish oil. Also, the present disclosure can exclude a composition, device, method, that comprises one or more terpenes. What can be excluded is a composition, device, method, that comprises one or more of peppermint oil, orange oil, lemon oil, cannabis oil, hemp oil, and so on. Also, what can be excluded is any composition, device, or method, that comprises one or more of alpha-bisabolol, bomeol, alpha-caryophyllene, betacaryophyllene, elemene (alpha, beta, gamma, or delta), limonene, camphene, camphor, delta-3-carene, caryophyllene oxide, alpha-cedreen, citral, eucalyptol, beta-eudesmol, eudesm-7(11)-en4-ol, farnesene, fenchol, alpha-guaiene, geraniol, guaiol, germacrene B, guaia-1(10)-11-diene, humulene, alpha-humulene, isobomeol, linalool, menthol, myrcene, alpha-myrcene, betamyrcene, nerol, cis-ocimene, trans-ocimene, alpha-phellandrene, alpha-pinene, beta-pinene, pulegone, sabinene, alpha-terpinene, alpha-terpineol, terpinolene, terpineol, thymol, trans-2-pinanol, selina-3,7(11)-diene, or valencene.

In device embodiments, a device of the present disclosure is substantially free of one or more of tetrahydrocannabinol (THC), tetrahydrocannabinolic acid (THC-a), and cannabichromene (CBC). Also, a device of the present disclosure is substantially free of each and every one of tetrahydrocannabinol (THC), tetrahydrocannabinolic acid (THC-a), and cannabichromene (CBC). In composition embodiments, a composition of the present disclosure is substantially free of one or more of tetrahydrocannabinol (THC), tetrahydrocannabinolic acid (THC-a), and cannabichromene (CBC). Also, a composition of the present disclosure is substantially free of each and every one of tetrahydrocannabinol (THC), tetrahydrocannabinolic acid (THC-a), and cannabichromene (CBC).

In one aspect, the term “substantially free” can mean that the quantity of one or more of THC, THC-a, and CBC, occurs at a molar quantity that is under 20%, under 15%, under 10%, under 5^{%}, under 4%, under 2%, under 1%, under 0.5%, under 0.1%, under 0.05%, or under 0.01%, that of CBD. In another aspect, the term “substantially free” can mean that the quantity of each and every one of THC, THC-a, CBN, and CBC, occurs at a molar quantity that is under 20%, under 15%, under 10%, under 5%, under 4%, under 2%, under 1%, under 0.5%, under 0.1%, under 0.05%, or under 0.01%, that of eugenol, capsaicin, cannabidiol (CBD), cannabidiolic acid (CBDA), diclofenac, or β-caryophyllene (BCAP).

The following methods of measurement take into account the physical nature of a composition and the physical nature of the container or matrix that comprises a composition. In measuring a composition that is “substantially free,” what can be measured is all compounds that are comprised by the composition, where the composition takes the form of an oil, a paste, a slurry, an adhesive, a powder, a solution, and the like, or that takes the form of a matrix, a reservoir, and impregnated fabric, a flask, a conduit, that holds, contains, absorbs, adsorbs, and the like, the oil, a paste, a slurry, an adhesive, a powder, a solution, and the like.

In embodiments, the present disclosure can exclude a reservoir-type device where backing does not directly contact reservoir; or where reservoir does not directly contact a hydrophilic porous membrane; or where hydrophilic porous membrane does not directly contact a release liner; or where reservoir does not contain all of: (1) a liquid carrier, (2) a gelling agent, and (3) one or more of eugenol, capsaicin, cannabidiol (CBD), cannabidiolic acid (CBDA), diclofenac, or β-caryophyllene (BCAP). Also, what can be excluded is a reservoir-type device that does not comprise all of the above.

In embodiments, what can be excluded is an adhesive polymer, or a device comprising an adhesive polymer, where the adhesive polymer reacts with amines. Also what can be excluded, is an adhesive polymer, or a device comprising an adhesive polymer, where the adhesive polymer has any free hydroxyl groups, where the adhesive polymer has over 1 free hydroxyl groups per 100 atoms of the adhesive polymer, where the adhesive polymer has over 5 free hydroxyl groups per 100 atoms of the adhesive polymer, where the adhesive polymer has over 10 free hydroxyl groups per 100 atoms of the adhesive polymer, where the adhesive polymer has over 20 free hydroxyl groups per 100 atoms of the adhesive polymer, and so on. For this exclusionary embodiment, the skilled artisan understands that any polymer consists of a large number of atoms, for example, about five thousand atoms.

In embodiments, what can be excluded is a monolith-type device where a backing is not in direct contact with a matrix of skin adhesive; where matrix of skin adhesive is not in direct contact with a releasable liner; where matrix does not comprise one or more of eugenol, capsaicin, cannabidiol (CBD), cannabidiolic acid (CBDA), diclofenac, or β-caryophyllene (BCAP); or all of the above.

What can also be excluded is a preparation, or a device comprising a preparation, where the preparation has over 1% gelling agent, over 2%, over 3%, over 4%, over 5%, over 6%, over 7%, over 8%, over 9%, over 10%, over 12%, over 14%, or over 16%, of gelling agent. Also, what can be excluded is a preparation, or a device comprising a preparation, where the preparation has under 1% gelling agent, under 2%, under 3%, under 4%, under 5%, under 6%, under 7%, under 8%, under 9%, under 10%, under 12%, under 14%, or under 16%, of gelling agent.

What can also be excluded is a preparation, or a device comprising a preparation, where the preparation has over 1% penetration enhancer, over 2%, over 3%, over 4%, over 5%, over 6%, over 7%, over 8%, over 9%, over 10%, over 12%, over 14%, or over 16%, of penetration enhancer. Also, what can be excluded is a preparation, or a device comprising a preparation, where the preparation has under 1% penetration enhancer, under 2%, under 3%, under 4%, under 5%, under 6%, under 7%, under 8%, under 9%, under 10%, under 12%, under 14%, or under 16%, of penetration enhancer.

In other embodiments, what can be excluded is a preparation, a composition, a device comprising a preparation, a device comprising a composition, where said preparation or composition has a eugenol, capsaicin, cannabidiol (CBD), cannabidiolic acid (CBDA), diclofenac, or β-caryophyllene (BCAP) (or combined weight of eugenol, capsaicin, cannabidiol (CBD), cannabidiolic acid (CBDA), diclofenac, and β-caryophyllene (BCAP)) content by weight of under 1%, under 2%, under 3%, under 4%, under 5%, under 6%, under 8%, under 10%, under 12%, under 14%, under 16%, under 18%, under 20%, under 25%, under 30%, under 35%, under 40%, under 45%, under 50%, under 55%, under 60%, under 65%, under 70%, under 75%, and so on. Also, what can be excluded is a preparation, a composition, a device comprising a preparation, a device comprising a composition, where said preparation or composition has a eugenol, capsaicin, cannabidiol (CBD), cannabidiolic acid (CBDA), diclofenac, or β-caryophyllene (BCAP) (or combined weight of eugenol, capsaicin, cannabidiol (CBD), cannabidiolic acid (CBDA), diclofenac, and β-caryophyllene (BCAP)) content by weight that is greater than 5%, greater than 6%, greater than 7%, greater than 8%, greater than 10%, greater than 12%, greater than 14%, greater than 16%, greater than 18%, greater than 20%, greater than 25%, greater than 30%, greater than 35%, greater than 40%, greater than 45%, greater than 50%, greater than 55%, greater than 60%, greater than 65%, greater than 70%, and so on. In embodiments, what can be excluded is a preparation, a composition, a device comprising a preparation, or a device comprising a composition, where the percent by weight is defined by one or more of the above “under” or “greater than” parameters. “Composition” can refer to, for example, matrix of a skin adhesive, or to fluid in hydrophilic porous membrane, and so on. Alternatively, the present disclosure can comprise one or more of the above compositions, as set forth by “under” parameters or “greater than” parameters.

Moreover, in embodiments what can be excluded is any device that does not include an occlusive system polymer film, that does not include a polyethylene occlusive polymer film, that does not include a PET occlusive polymer film, that does not include an occlusive polymer film made of both polyethylene and PET. Also, what can be excluded is a device that has an overlay patch, and a device that does not comprise an overlay patch.

In embodiments, polar organic liquid can comprise, or can exclude, one or more of methanol, ethanol, propanol, isopropanol, butanol, pentanol, acetic acid, propionic acid, butyric acid, valeric acid, caproic acid, caprylic acid, capric acid, lauric acid, myristic acid palmitic acid, stearic acid, palmitoleic acid, oleic acid, linoleic acid, linolenic acid, linear alkanes of 5, 6, 7, 8, 9, 10, 11, 12, 13, 14, 15, 16, 17, 18, 19, 20, 21, 22, 23, 24, or more carbons, branched chain alkanes with a backbone of 5, 6, 7, 8, 9, 10, 11, 12, 13, 14, 15, 16, 17, 18, 19, 20, 21, 22, 23, 24, or more carbons, linear alkenes (olefins) of 5, 6, 7, 8, 9, 10, 11, 12, 13, 14, 15, 16, 17, 18, 19, 20, 21, 22, 23, 24, or more carbons, branched chain alkenes (olefins) with a backbone of 5, 6, 7, 8, 9, 10, 11, 12, 13, 14, 15, 16, 17, 18, 19, 20, 21, 22, 23, 24, or more carbons, and so on. Alternatively, the present disclosure can comprise one or more of the above polar organic liquids.

### EXAMPLES

Monolithic Patch Embodiments, as Described in US2017/0071870 of Weimann.

The following writing is from US2017/0071870 (Ser. No. 15/265,823) of Weimann, which is incorporated herein by reference in its entirety.

Transdermal monolithic anti-pain drug patch formulation: Adhesive polymer: 60-95, CBD: 5-20 Penetration enhancer: 0-20, Adhesive polymer: Acrylate from Henkel, Silicone from Dow Corning. PIB from BASF CBD: pure crystalline powder Penetration enhancer: Oleic acid, isopropyl palmitate (IPP), dimethylsulfoxide (DMSO), 1,2-propylene glycol (1,2-PG), isopropyl myristate (IPM). In this example, the dry adhesive matrix is 30-50 micrometers thick. The area of the patch can be square or oval. The best size of the patch is 20 cm² by 40 cm².

In a monolithic design, a release liner is coated with a mixture of CBD and a PIB or amine-compatible silicone skin adhesive laminated to the backing material. How the Monolithic CBD Patch Works: Step 1. CBD is dissolved in ethyl alcohol or 1,2 PG and mixed into the adhesive solution and penetration enhancer is added if needed. Step 2. Adhesive mix is dispensed on the release liner by means of “knife-over-roll” coating method and dried in the oven at drying time from 1 min to 3 min or until all residual solvents are below 50 ppm. Step 3. Dried adhesive film is laminated to the backing film by means of nipping and edges are slit for further die cutting of the patches. Step 4. The laminate is placed on the die cutting machine and proper size patches are cut and later packaged in the pouches and boxes.

### Monolithic Patch Embodiments

In the present disclosure, transdermal monolithic anti menstrual cramping drug patch formulations can take the form of: Adhesive polymer: 60-95%, one or more of eugenol, capsaicin, cannabidiol (CBD), cannabidiolic acid (CBDA), diclofenac, or β-caryophyllene (BCAP): 5-20 Penetration enhancer: 0-20, Adhesive polymer: Acrylate from Henkel, Silicone from Dow Corning, PIB from BASF, Penetration enhancer: Oleic acid, isopropyl palmitate (IPP), dimethylsulfoxide (DMSO), 1,2-propylene glycol (1,2-PG), isopropyl myristate (IPM), 2-(2-Ethoxyethoxy)ethanol (Transcutol®). In this example, the dry adhesive matrix is 25-75 micrometers thick. The area of the patch can be square, rectangular, or oval. The best size of the patch is about 50 cm².

How Anti-Pain Drugs are Delivered from Patch Formulation to the Body Through the Skin.

The present disclosure encompasses dermal patches, systems, devices, formulations, and compositions, wherein the formulation or composition contains the indicated anti-pain drugs. Said formulation or composition can contain one, two, three, four, or more different anti pain drugs, as listed below.

(A) With eugenol, capsaicin, cannabidiol (CBD), cannabidiolic acid (CBDA), diclofenac, or β-caryophyllene (BCAP), or

Where the mixture includes a cannabinoid, the cannabinoid is CBD: (B) With a mixture of exactly two of eugenol, capsaicin, CBD, diclofenac, and β-caryophyllene (BCAP).

Where the mixture includes a cannabinoid, the cannabinoid is CBDa: (B) With a mixture of exactly two of eugenol, capsaicin, CBDA, diclofenac, and β-caryophyllene (BCAP).

Mixtures of three drugs. (C) Where the mixture includes a cannabinoid, the cannabinoid is CBD: With a mixture exactly three of eugenol, capsaicin, CBD, diclofenac, and β-caryophyllene (BCAP).

Mixtures of three drugs. (C) Where the mixture includes a cannabinoid, the cannabinoid is CBDA: With a mixture exactly three of eugenol, capsaicin, CBDA, diclofenac, and β-caryophyllene (BCAP).

Mixtures of four drugs. (D) Where the mixture includes a cannabinoid, the cannabinoid is CBD: With a mixture exactly four of eugenol, capsaicin, CBD, diclofenac, and β-caryophyllene (BCAP).

Mixtures of four drugs. (D) Where the mixture includes a cannabinoid, the cannabinoid is CBDA: With a mixture exactly four of eugenol, capsaicin, CBDA, diclofenac, and β-caryophyllene (BCAP).

Mixtures of five drugs. (E) Where the mixture includes a cannabinoid, the cannabinoid is CBD: With a mixture exactly five of eugenol, capsaicin, CBD, diclofenac, and β-caryophyllene (BCAP).

Mixtures of five drugs. (E) Where the mixture includes a cannabinoid, the cannabinoid is CBDA: With a mixture exactly five of eugenol, capsaicin, CBDA, diclofenac, and β-caryophyllene (BCAP).

Mixtures of six drugs. (E) Where the mixture contains exactly six of eugenol, capsaicin, CBD, CBDA, diclofenac, and β-caryophyllene (BCAP).

In embodiments, any of the above mixtures can be formulated so that both CBD and CBDa are present in the mixture (here, the formulation is made without regard to the statement that the formulation contains two anti-pain drugs, three anti-pain drugs, four anti-pain drugs, five anti-pain drugs, or six anti-pain drugs.

In embodiments, any of the above mixtures can be formulated so that neither CBD nor CBDa is present in the mixture (here, the formulation is made without regard to the statement that the formulation contains two anti-pain drugs, three anti-pain drugs, four anti-pain drugs, five anti-pain drugs, or six anti-pain drugs.

Skin Adhesives

In a first monolithic-style device, the skin adhesive is preferably a polyisobutylene adhesive having a weight-average molecular weight (Mw) from about 100,000 to 400,000 Daltons, preferably from about 150,000 to 350,000 Daltons, and more preferably from about 200,000 to 300,000 Daltons. These molecular weight ranges are not meant to be limiting, and other embodiments of these inventions may deviate from these stated Mw ranges because of the blending required to match adhesion, tack, and drug diffusion.

In a second monolithic-style device, a skin adhesive is mixed with one or more anti pain drugs, to define a monolithic mixture of adhesive and said anti-pain drugs. The skin adhesive is coated on a backing that is preferably occlusive. The skin adhesive is preferably an amine-compatible silicone adhesive.

In a third monolithic-style device, a skin adhesive is mixed with one or more anti pain drugs, to define a monolithic mixture of adhesive and said anti-pain drugs. The skin adhesive is coated on a backing that is preferably occlusive. The skin adhesive is preferably a polyacrylate adhesive.

Manufacturing Method for Monolithic Patch.

Anti-pain drugs are dissolved in ethyl alcohol, methyl alcohol, 1,2-propylene glycol (1,2 PG), or other organic solvent and mixed into the adhesive solution and penetration enhancer is added if needed. Adhesive mix is dispensed on the release liner by means of “knife-over-roll” coating method and dried in the oven at drying time from 1 min to 3 min or until all residual solvents are below 50 ppm. Dried adhesive film is laminated to the backing film by means of nipping and edges are slit for further die cutting of the patches. The laminate is placed on the die cutting machine and proper size patches are cut and later packaged in the pouches and boxes.

The skin adhesive comprising matrix 44 preferably comprises at least one of an acrylate pressure sensitive adhesive, a polyisobutylene pressure sensitive adhesive, and an amine-compatible silicone pressure sensitive adhesive. Suitable acrylate adhesives include DuroTak 87-2074. Suitable polyisobutylene adhesives include those having a weight-average molecular weight (Mw) from about 100,000 to 400,000 Daltons, preferably from about 150,000 to 350,000 Daltons, and more preferably from about 200,000 to 300,000 Daltons. These molecular weight ranges are not meant to be limiting, and other embodiments of these inventions may deviate from these stated Mw ranges because of the blending required to match adhesion, tack, and drug diffusion.

Matrix 44 preferably comprises a polyisobutylene adhesive having a viscosity-average molecular weight as described above and an adhesion/viscosity modifier. The adhesion/viscosity modifier is preferably a mineral oil or silicone fluid present in an amount ranging from about one (1) to about ten (10) percent by weight of matrix 44, more preferably from about two (2) to about six (6) percent by weight of matrix 44, and still more preferably from about three (3) to about four (4) percent by weight of the matrix 44. Mineral oils that are suitable for use as the adhesion/viscosity modifier have a molecular weight ranging from 100 to about 10,000 Daltons, more preferably from about 200 to about 6000 Daltons, and even more preferably from about 350 Daltons to about 4500 Daltons. Silicone fluids that are suitable for use as the adhesion/viscosity modifier preferably comprise —OH end-capped polydimethylsiloxanes having a kinematic viscosity at 20.degree. C. ranging from about 100 cSt to about 1000 cSt. Commercially available silicone fluids that may be used as the adhesion/viscosity modifier include the Dow Corning Q7-9120 fluids, which are available in kinematic viscosities (at 20.degree. C.) of 20, 100, 350, 1000, and 12,500 cSt. In preferred examples of silicone adhesion/viscosity modifier, the Q7-9120 100 cSt or 1000 cSt (or mixtures thereof) are used.

Preferred polyisobutylene adhesives are not supplied with mineral oil. In certain preferred examples, the polyisobutylene component of matrix 44 is a Vistanex LM polyisobutylene adhesive. In other preferred examples, the polyisobutylene component of matrix 44 is an Oppanol B or N grade polyisobutylene adhesive supplied by BASF. In other preferred examples, the polyisobutylene component of matrix 44 is a mixture of Oppanol B or N grade polyisobutylene adhesives supplied by BASF.

In another example, the adhesive component of matrix 44 may comprise a blend of acrylic adhesive and polyisobutylene adhesive, and preferably, a blend of an acrylic adhesive and a polyisobutylene adhesive having the weight-average molecular weight (Mw) described above (from about 100,000 to 400,000 Daltons, preferably from about 150,000 to 350,000 Daltons, and more preferably from about 200,000 to 300,000 Daltons.). When acrylic adhesives are combined with such polyisobutylene adhesives, the amount of acrylic adhesive by weight of the total amount of adhesive in matrix 44 is preferably from about one percent to about 50 percent. In one example, the adhesive component of matrix 44 comprises 80 percent Oppanol PIB adhesive (or mixture of different Oppanol PIB adhesives) by weight of the total amount of adhesive in matrix 44 and twenty percent Durotak 87-2516 by weight of the total amount of adhesive in matrix 44.

Penetration Enhancers

Monolithic device may also include one or more penetration enhancers, including oleic acid, 2-(2-Ethoxyethoxy)ethanol (Transcutol®), isopropyl palmitate (IPP), DMSO, 1,2 propylene glycol, and isopropyl myristate (IPM). The amount of penetration enhancer preferably ranges from zero to about ten (10) percent by weight of the matrix. In an exclusionary embodiment, the present disclosure can exclude any patch or any formulation that has more than one type of penetration enhancer.

The skin contact area of the device is preferably at least about 10 cm², more preferably at least about 15 cm², and still more preferably at least about 25 cm². At the same time, the skin contact area of the device is preferably no more than about 200 cm², preferably no more than about 150 cm², and still more preferably no more than about 100 cm². At a given flux rate, the skin contact area may be selected to achieve the desired daily dose of anti pain drug (or the dose over whatever time period is of therapeutic interest).

Without implying any limitation on the present invention, knife coating is a process by which a thin liquid coating is formed on a continuous web by the application of an excess of coating liquid which is subsequently metered by a rigid knife held in close proximity to a rigidly supported web. The thickness of the coating depends primarily on the clearance, or gap, between the knife and the web, and upon the geometry of the gap (bevel angle, length). Roll coating is a process by which a thin liquid film is formed on a continuous web by use of two or more rotating rolls, such that the fluid flow in a small gap between a pair of rotating rolls is the primary factor controlling the thickness and uniformity of the coated film. The thickness of the coating depends primarily on the gap between adjacent rolls and their relative speeds. Two basic types of roll coaters are distinguished by the relative direction of roll surface motion in the gap: in forward roll coating the roll surfaces move in the same direction and in reverse roll coating they move in opposite directions. In terms of the flow fields, knife coating is a subset of forward roll coating where one surface is stationary. See, Coyle, D. J (1997) Knife and Roll Coating in Liquid Film Coating (ed. S. F. Kistler and P. M. Schweizer). Chapman & Hall, London; W. Rehnby, M. Gustafsson, M. Skrifvars (June 2008) Conference Paper, Coating of Textile Fabrics with Conductive Polymers for Smart Textile Applications, pages 100-103.

### Reservoir Patch Embodiments

Reservoir Patch Manufacturing Method.

The present inventor has used the following manufacturing method for reservoir patch, where the method used the indicated stages:
STAGE 1. Gel dispenser. Dispensing active gel solution on membrane. At this point, the stage of manufacture can be represented by an isolated blob of active gel.
STAGE 2. Heat seal press. Gel is covered with heat sealable film and heat seal around the gel. At this point, the stage of manufacture can be represented by a blob in the center, surrounded by a layer of film.
STAGE 3. Kiss cut press. Kiss cutting along the periphery of the heat seal ring. At this point, the stage of manufacture can be represented by a blob at the center, surrounded by a layer of film and where the interior side of the layer is intact and where the exterior layer is perforated. According to one source, “Kiss cutting is . . . a method for providing a converted adhesive tape solution. During the kiss-cutting process, the perimeter of each piece is stamped out by a sharp metal die or by a precision laser beam . . . the cut does not penetrate the piece's backing material (liner). Even though the die or laser makes a clean cut all the way through the usable portion of the material, it merely “kisses” the liner sheet. This allows single or multiple adhesive materials to remain on a liner sheet or roll until the end user is ready to remove them.” (CAN-DO® National Tape, Nashville, TN). According to another source, “Laser kiss cutting is used to cut the top layer of a material without cutting through an attached material. Sticker labels are a good example of laser kiss cutting in action. In this process, the outline of the label can be cut without cutting the release or backing material. Typically, CO2 lasers are used for kiss cutting applications. Laser kiss cutting can also be combined with perforating or “through cutting” on a single application.” (Preco Kansas, Lenexa, Kansas).
STAGE 4. Kiss cut press. Reservoir is covered with overlay film, and kiss cut along the overlay periphery. At this point in the manufacture, the article of manufacture obtained in STAGE 3 is surrounded by another layer, where this layer is the overlay film.
STAGE 5. Cut through press. Patch is cut through the overextended release liner, for easier peel. At this point, the article of manufacture resembles that obtained in STAGE 4, except that the article of manufacture is chopped into segments, where each segment is suitable for attaching to the skin of a human patient or a human consumer.

Additional Descriptions of Reservoir Patch.

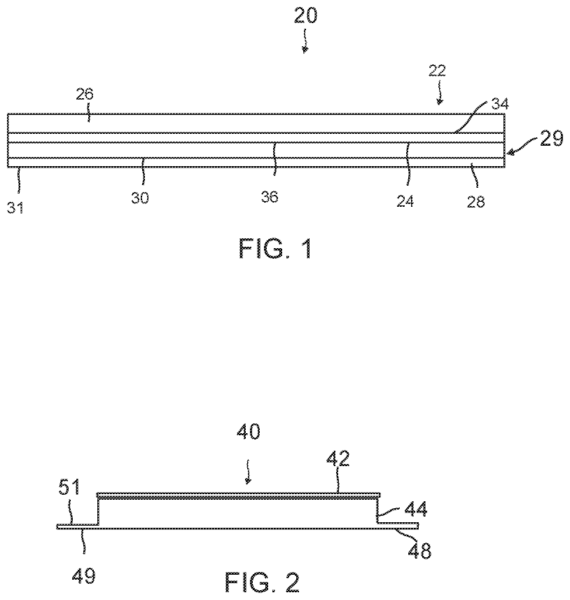

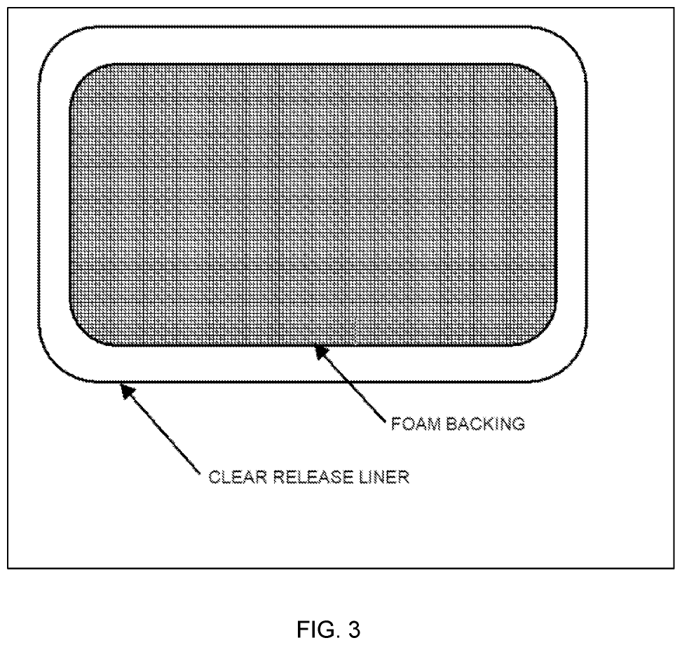

The following writing, and structure numbers, are from US2017/0071870 (Ser. No. 15/265,823) of Weimann, which is incorporated herein by reference in its entirety. Referring to FIG. 1 of US2017/0071870, a reservoir-style transdermal delivery device 20 for the transdermal delivery of CBD is depicted. Reservoir-style transdermal delivery device 20 comprises a backing 22 and a hydrophilic, porous membrane 24. The backing 22 and hydrophilic, porous membrane 24 are attached to one another so as to define a closed volume which acts as a reservoir 26. A preparation 27 (or formula or composition) that comprises one or more anti-pain drugs is deposited in the reservoir 26. First side 34 of the hydrophilic, porous membrane 24 is in contact with the preparation 27. A second side 36 of the hydrophilic, porous membrane 24 faces away from backing 22 and is coated with a skin adhesive 30. The skin adhesive 30 is preferably formulated to adhere the device 20 to the user's skin for a period of no less than about 24 hours while avoiding appreciable skin irritation to the user's skin. Preferred skin adhesives 30 include amine-compatible, silicone, pressure sensitive adhesives. In certain examples, an amine compatible silicone skin adhesive 30 is provided which comprises a trimethylsiloxy end-capped reaction product of a silanol end-blocked polydimethylsiloxane and a silicate resin. The skin adhesive is preferably provided as an organic solvent solution comprising from about 50 percent to about 70 percent by weight of solid adhesive in an organic solvent like heptane or ethyl acetate and having a viscosity at 20 degrees C. of from about 400 mPa-s to about 1300 mPa-s, preferably from about 450 mPa-s to about 1250 mPa-s, and more preferably from about 500 mPa-s to about 1200 mPa-s.

A first surface of a release liner is releasably adhered to skin adhesive, and a second surface of release liner faces away from skin adhesive. Suitable release liners include occlusive polymeric films, such as polyester, polypropylene, coated with a release coating that is releasably adherable to silicone, polyisobutylene, and silicone adhesives. Suitable release coatings on first surface of release liner include fluoropolymers and silicone polymers. Commercially-available, coated release liners that are suitable for use as release liner 28 include Scotchpak 1022, 9741, 9744, 9748, and 9755 supplied by 3M of Minneapolis, Minn., and FRA 314 and 315 supplied by Fox River Co. To use the reservoir transdermal device, the release liner is peeled away from skin adhesive, thereby exposing skin adhesive, and the device is applied so that the skin adhesive contacts the user's skin.

Suitable examples of such amine-compatible silicone adhesives include the BIO-PSA 7-4301 and 7-4302 skin adhesives supplied by Dow Corning. BIO-PSA 7-4301 is a high tack, amine-compatible silicone adhesive in heptane available with a solids content of 60 percent and 70 percent and corresponding viscosities at 20 degrees C. of 450 mPa-s and 1600 mPa-s. BIO-PSA 7-4302 is a high tack, amine-compatible silicone adhesive in ethyl acetate with a solids content of 60 percent by weight and a viscosity of 1200 mPa-s at 20 degrees C. The skin adhesive 30 is coated to a thickness per unit area on the membrane 24 that is preferably from about 20 to about 80 g/m², more preferably from about 40-70 g/m², and still more preferably from about 50-60 g/m².

Hydrophilic, porous membrane preferably has a mean flow pore size of no more than about 1 micron, preferably not more than about 0.8 microns, still more preferably no more than about 0.4 microns, and even more preferably no more than about 0.2 microns. At the same time, porous membrane 24 preferably has a mean flow pore size of no less than about 0.02 microns, more preferably no less than about 0.04 microns, still more preferably no less than about 0.06 microns, and even more preferably no less than about 0.08 microns. The mean flow pore size may be determined in accordance with the method set forth at page 17, line 22 to page 18, line 4 of published PCT Application WO2010072233, the entirety of which is hereby incorporated by reference.

In the same or other examples, hydrophilic porous membrane preferably has a porosity of at least about 60 percent, more preferably at least about 65 percent, and still more preferably at least about 70 percent. At the same time, hydrophilic porous membrane preferably has a porosity of no more than about 90 percent, more preferably no more than about 85 percent, and still more preferably no more than about 80 percent. Porosity values may be calculated as described at page 7, lines 24 to 27 of WO2010072233.

In the same or other examples, hydrophilic porous membrane preferably has a thickness of no more than about 50 microns, preferably no more than about 40 microns, and even more preferably no more than about 35 microns. At the same time, hydrophilic porous membrane 24 preferably has a thickness of no less than about 10 microns, more preferably no less than about 20 microns, and still more preferably no less than about 25 microns. Membrane thicknesses may be determined as described at page 18, lines 19-21 of WO2010072233.

In the same or other examples, hydrophilic porous membrane 24 preferably has an air permeability as determined by the Gurley Test Method (according to ISO 5636-5) that is preferably at least about 10 sec/50 ml, more preferably at least about 20 sec/50 ml, and still more preferably at least about 25 sec/50 ml. At the same time, hydrophilic porous membrane 24 preferably has an air permeability of no more than about 50 sec/50 ml, more preferably no more than about 40 sec/50 ml, and still more preferably no more than about 35 sec/50 ml.

In the same or other examples, hydrophilic porous membrane 24 preferably has a tensile strength in the machine direction as determined by ASTM D882-12 that is preferably at least about 10 MPa, more preferably at least about 15 MPa, and still more preferably at least about 20 MPa. In the same or other examples, the hydrophilic porous membrane 24 preferably has a percent elongation in the machine direction as determined by ASTM D882-12 that is preferably at least about 10 percent, more preferably at least about 15 percent, and still more preferably at least about 20 percent.

Hydrophilic porous membrane 24 preferably comprises at least one polymeric material. In one example, hydrophilic porous membrane 24 comprises a polyolefin polymer and a hydrophilic component that comprises a hydrophilic polymer and optionally, a surfactant. As used herein, the term “hydrophilic” when used to describe a porous membrane refers to a membrane that at 20 degrees C. provides a water flux for demineralized water through the membrane of at least 0.5 liters/(m2 hbar).

The content of the polyolefin polymer is preferably less than or equal to 98 percent by weight based on the total dry weight of the membrane, and the content of the hydrophilic component(s) is preferably at least 2 weight percent based on the total dry weight of the membrane. In certain preferred examples, the membrane is formed by combining the polyolefin polymer with the hydrophilic components(s) and optional additives with a solvent to form a blend in the form of a gel, a solution, or a homogeneous mixture, followed by extruding the blend. Suitable polyolefins (such as polyethylene), hydrophilic components, and additives are described in WO2010072233.

In another preferred embodiment, device comprises transdermal patch formulation comprising a reservoir in the shape of a “ravioli” constructed with microporous hydrophilic or hydrophobic membrane on one side and occlusive film on other side.

In embodiments, device comprises transdermal reservoir patch formulation as thixotropic alcohol or alcohol/water solution gelled with hydroxyalkyl cellulose containing CBD at high concentration ranging from 1% to 50% CBD Moreover, device comprises transdermal reservoir patch formulation comprising a reservoir containing thixotropic alcohol or alcohol/water solution gelled with hydroxyalkyl cellulose and containing one or more of eugenol, capsaicin, cannabidiol (CBD), cannabidiolic acid (CBDA), diclofenac, or β-caryophyllene (BCAP) at a high concentration, ranging from 1% to 50% and skin penetration enhancers in a concentration range of 0% to 10%.

What is also encompassed, is transdermal patch formulation comprising a reservoir in shape of “ravioli” constructed with microporous hydrophilic or hydrophobic membrane on one side and occlusive film on other side where the microporous membrane is coated with thin layer of silicone adhesive. In delivery embodiments, reservoir patch of 20 cm² is capable of systemically delivering one or more of eugenol, capsaicin, cannabidiol (CBD), cannabidiolic acid (CBDA), diclofenac, or β-caryophyllene (BCAP) at about 0.5 mg/day, about 1.0 mg/day, about 1.5 mg/day, about 2.0 mg/day, about 5.0 mg/day, about 10 mg/day, about 15 mg/day, about 20 mg/day, about 25 mg/day, about 30 mg/day, about 35 mg/day, about 40 mg/day, and the like.

In other delivery embodiments, reservoir patch of 20 cm² is capable of systemically delivering one or more of eugenol, capsaicin, cannabidiol (CBD), cannabidiolic acid (CBDA), diclofenac, or β-caryophyllene (BCAP) at least 0.5 mg/day, at least 1.0 mg/day, at least 1.5 mg/day, at least 2.0 mg/day, at least 5.0 mg/day, at least 10 mg/day, at least 15 mg/day, at least 20 mg/day, about 25 mg/day, about 30 mg/day, at least 35 mg/day, at least 40 mg/day, and so on.

Adhesives, Tackifiers, and Enhances, with Reference to Data in US2017/0071870 of Weimann.

Adding acrylic adhesive with non-functionality Durotak 87-900A and adhesive with only OH-functionality Durotak 87-2510 compared with PIB with enhancers azone and oleic acid and DMSO. Azone promotes hydrophilic and water-based drugs passage into the skin. It is used in cosmetics as a skin penetration enhancer.

PIB adhesive with tackifiers that improve adhesion to skin using acrylic pressure sensitive adhesive mixed in at 1-50%. Also use of cycloaliphatic hydrocarbon resins such as Escorez 5300® resins from ExxonMobil. The disclosure provides a graph showing peel strength from skin.

PIB adhesive with enhancers: at 3% of azone or oleic acid double the transdermal delivery from PIB. The disclosure provides a graph showing transdermal flux.

Regarding peel strength of adhesives, the inventor used judgment and estimation for preparing the patch with 26% of tackifying resin. Escorez 5400 has been used in many adhesives but not in PIB. The inventor was the first to use it, and the inventor's results demonstrated that it worked as a tackifier. The inventor estimated the peel strength of the formulation with tackifier and without tackifier, by peeling them both from the inventor's own arm skin. To obtain saturation concentration of CBD in the PIB adhesive with 26% of tackifying resin Escorez 5400 the inventor needed to add more CBD to reach turbidity. The concentration of the saturation was about 18% CBD in PIB adhesive. The transdermal flux of CBD however did not increase. It was the same as the flux from PIB saturated with CBD without tackifier.

The benefit of using tackifier would be to use it in formulations that may contain additional active ingredients together with cannabinoids that might adversely affect adhesion of the patch to skin like addition of liquid terpenes.

The inventor found that the Escorez 5400 tackifier increased solubility of CBD in PIB adhesive, where this permits greater concentrations of CBD to be used in manufacturing dermal patch. This is expected to result in thus greater flux of CBD to a patient, where the dermal patch is in contact with the skin of a patient.

In embodiments, the present disclosure provides a patch with about 26% tackifying resin, that is, about 26% tackifier, about 12%, about 14%, about 16%, about 18%, about 20%, about 22%, about 24%, about 26%, about 28%, about 30%, about 32%, or about 34% tackifier, and the like. Also, the present invention provides a patch with 12%, 14%, 16%, 18%, 20%, 22%, 24%, 26%, 28%, 30%, 32%, or 34% tackifier, and the like. In range embodiments, the present disclosure provides a patch with tackifier in the range of, 10-12% tackifier, 12-14%, 14-16%, 16-18%, 18-20%, 20-22%, 22-24%, 24-26%, 26-28%, 28-30%, 30-32%, or 32-34% tackifier, and the like, or a range consisting of the sum of any two of these consecutive ranges, or the sum of any three of these consecutive ranges, or the sum of any four of these consecutive ranges, and the like. In exclusionary embodiments, the present disclosure can exclude a patch with any of the above tackifier values, with “about” tackifier values, or with any of the above tackifier ranges.

### Example. Chemistry of Acrylic Adhesives and Chemistry of Tackifiers of the Present Disclosure

Duro-Tak® 87-2516 is an acrylic copolymer adhesive containing EHA, vinyl acetate, and hydroxyethylacrylate. EHA is 2-ethylhexyl acrylate (see, U.S. Pat. No. 5,783,208 of Venkateshwaran). Duro-Tak® 87-2516 is an acrylate-vinyl acetate copolymer with a hydroxyl group (see, Zhao, Park, Kim, Lee (2002) Drug Devel. Industrial Pharmacol. 28:1125-1131). Duro-Tak® 87-2516 has viscosity of 4350 cp at 41.5% solids (see, US2006/173,124 of Paul). Duro-Tak® 87-2516 is hydroxyfunctional and crosslinked (see, US2002/0058068 of Houze). Duro-Tak® 87-2516 is an acrylate-vinyl acetate self-curing pressure-sensitive adhesive in an organic solvent (see, US2006/0121102 of Chiang).

Duro-Tak 87-4287 is a copolymer with 2-ethylhexyl acrylate as the main repeating monomer unit. Duro-Tak 87-4287 is a copolymer with vinyl acetate and contains OH— functional groups as 2-hydroxyethyl acrylate is also part of the polymer composition (Wolff (2014) Pharm. Res. 31:2186-2202). [00352] Duro-Tak® 87-2287 is a polyacrylate adhesive. According to U.S. Pat. No. 5,693,335 of Xia, “Duro-Tak 87-2287 is a solution of polyacrylate adhesive available from National Starch and Chemical Co. Its monomer composition is: vinyl acetate, 28%; 2-ethylhexyl acrylate, 67%; hydroxyethyl acrylate, 4.9% glycidyl methacrylate, 0.1%. It contains no crosslinking agent. It is available as a 50% solids solution in ethyl acetate.” See also, U.S. Pat. No. 6,071,531 of Jona. According to U.S. Pat. No. 5,780,050 of Jain, Duro-Tak® 87-2287 is an acrylic adhesive free of acid functional groups. According to US2009/0258061 of Hwang, “Duro-Tak® 87-2287 is an adhesive is derived from a monomer composition of vinyl acetate, 28%; 2-ethylhexyl acrylate, 67%; hydroxyethyl acrylate, 4.9%; and glycidyl methacrylate, 0.1%, see U.S. Pat. No. 5,693,335.”

DuroTak® 87-900A is an acrylic pressure-sensitive adhesive that comprises 2-ethylhexyl acrylate, butyl acetate, t-octyl acrylamide, and methyl methacrylate. This list of chemicals was accepted, as a substitute for “DuroTak® 87-900A” by the patent examiner in file history of US2009/0297590 of Yamagi. According to a Product Selection Guide, DuroTak® 87-900A has no crosslinker, no vinyl acetate, 43% solids, viscosity of 1800 cP (see, DURO-TAK and GELVA Transdermal Pressure Sensitive Adhesives. Product Selection Guide (2013) Henkel Corp., Bridgewater, NJ (2 pages)). According to Wolff (2014) Pharm. Res. 31:2186-2202, Duro-Tak 87-900A is, “Duro-Tak 87-900A . . . have 2-ethylhexyl acrylate as the main repeating monomer unit . . . Duro-Tak 87-900A contains besides 2-ethylhexyl acrylate, butyl acrylate, methyl methacrylate and tertiary-octyl acrylamide units.” See also, para. 0031 of Yamagi US2009/0297590. Duro-Tak 87-900A contains 2-ethylhexyl acrylate as the main repeating monomer unit, and also contains butyl acrylate, methyl methacrylate and tertiary-octyl acrylamide units (Wolff (2014) Pharm. Res. 31:2186-2202).

Duro-Tak® 87-2510 has been described as, “copolymer: acrylate; functional group: OH; 40.5% solution of non-crosslinking acrylic copolymer, 4500 cps, solubility parameter 16.” (see, Kim, Gwak, Chun (2014) Arch. Pharm. Res. 27:763-768). [00355] Escorez® 5400 is described as, “dicyclopentadiene (DCPD) resin” (see, U.S. Pat. No. 9,296,930 of Hu); “hydrogenated polycyclopentadiene resin” (see, U.S. Pat. No. 9,039,862 of Lotz); a “hydrocarbon tackifying resin, having a molecular weight of about 400 grams/mole, a softening point of 103 degrees C., and a glass transition temperature of about 50 degrees C.” (see, U.S. Pat. No. 9,074,087 of Chen); a “cycloaliphatic hydrocarbon tackifying resin having a ring and ball softening point from about 100 degrees C. to about 106 degrees C.” (see, U.S. Pat. No. 9,803,113 of Tse). [00356] Escorez® 5400 has the following characteristics: softening point 218.1 degrees F., initial color: 0.6 YI; thermal color stability: 5 hours, 347 degrees F. (175 degrees C.) 6.4 YI, melt viscosity: 320 degrees F. (160 degrees C.) of 800 cP; molecular weight (number average; Mn) 400 g/mol; molecular weight (Mw) 670 g/mol; glass transition temperature (Tg): 126 degrees F. (Product Datasheet, ExxonMobil, Escorez® 5400 Tackifying Resin).

1. A pharmaceutical formulation that, when applied to the skin, is capable of reducing menstrual pain, wherein the composition comprises at least one of eugenol, capsaicin, cannabidiol (CBD), cannabidiolic acid (CBDA), diclofenac, and β-caryophyllene (BCAP), wherein said pharmaceutical formulation further may comprise of a penetration enhancer.

2. The pharmaceutical formulation of claim 1, wherein said penetration enhancer is one of azone, oleic acid, dihydromyricetin, limonene, dimethylsulfoxide (DMSO), 1,2-propylene glycol, or isopropyl myristate.

3. The pharmaceutical formulation of claim 1 that excludes one or more of opioids, muscle relaxants, acetaminophen, corticosteroids, antianxiety drugs, antidepressants, and anticonvulsant drugs.

4. The pharmaceutical formulation of claim 1, that comprises one of the following lists of chemical ingredients:
1) eugenol, capsaicin, and cannabidiol (CBD), but without CBDa,
2) eugenol, capsaicin, cannabidiol (CBD), and diclofenac, but without CBDa,
3) eugenol, capsaicin, cannabidiol (CBD), but without diclofenac and without CBDa,
4) eugenol, capsaicin, and diclofenac,
5) eugenol, capsaicin, without any diclofenac and without any cannabidiol (CBD),
6) eugenol, capsaicin, cannabidiol (CBD), cannabidiolic acid (CBDa),
7) eugenol, capsaicin, cannabidiol (CBD), cannabidiolic acid (CBDa), and diclofenac,
8) eugenol, capsaicin, cannabidiol (CBD), cannabidiolic acid (CBDa), but without diclofenac,
9) eugenol, capsaicin, without any diclofenac and without any cannabidiol (CBD),
10) eugenol, capsaicin, cannabidiolic acid (CBDa), but without CBD,
11) eugenol, capsaicin, cannabidiolic acid (CBDa), and diclofenac, but without CBD,
12) eugenol, capsaicin, cannabidiolic acid (CBDa), but without diclofenac, and without CBD,
13) eugenol, capsaicin, and diclofenac, but without CBD,
14) eugenol, capsaicin, and β-caryophyllene (BCAP),
15) eugenol, capsaicin, β-caryophyllene (BCAP), and diclofenac,
16) eugenol, capsaicin, β-caryophyllene (BCAP), but without diclofenac,
17) eugenol, capsaicin, without any diclofenac and without any β-caryophyllene (BCAP),
18) eugenol, capsaicin, β-caryophyllene (BCAP), cannabidiol (CBD),
19) eugenol, capsaicin, β-caryophyllene (BCAP), cannabidiolic acid (CBDa),
20) eugenol, capsaicin, β-caryophyllene (BCAP), cannabidiol (CBD), and diclofenac,
21) eugenol, capsaicin, β-caryophyllene (BCAP), cannabidiolic acid (CBDA), and diclofenac,
22) eugenol, capsaicin, β-caryophyllene (BCAP), cannabidiol (CBD), cannabidiolic acid (CBDA), and diclofenac,
23) eugenol, capsaicin, without any diclofenac and without any β-caryophyllene (BCAP),
24) eugenol, capsaicin, cannabidiolic acid (CBDa), but without β-caryophyllene (BCAP),
25) eugenol, capsaicin, cannabidiolic acid (CBDa), and diclofenac, but without (β-caryophyllene (BCAP),
26) eugenol, capsaicin, cannabidiolic acid (CBDa), but without diclofenac, and without (β-caryophyllene (BCAP),
27) eugenol, capsaicin, and diclofenac, but without β-caryophyllene (BCAP),
28) eugenol, capsaicin, without any diclofenac and without any β-caryophyllene (BCAP).
29) Any combination of one or more of eugenol, capsaicin, cannabidiol (CBD), cannabidiolic acid (CBDA), diclofenac, and β-caryophyllene (BCAP).

5. The pharmaceutical formulation of claim 1, that includes one or more of a polyisobutylene (PIB) adhesive, ascorbyl palmitate, a tackifier in the form of a resin, and one or more permeation enhancers.

6. A dermal patch comprising a pharmaceutical formulation capable of reducing menstrual pain when applied to the skin, wherein the composition comprises one or more of eugenol, capsaicin, cannabidiol (CBD), cannabidiolic acid (CBDA), diclofenac, and β-caryophyllene (BCAP).

7. The dermal patch of claim 6, wherein the menstrual pain results from primary dysmenorrhea or from secondary dysmenorrhea.

8. The dermal patch of claim 6, that is a reservoir-style dermal patch.

9. The dermal patch of claim 6, that is a monolithic-style dermal patch.

10. The dermal patch of claim 6, that comprises a flexible tan polyethylene foam backing layer on a clear polyethylene terephthalate (PET) release liner, wherein the foam backing is the part of the patch that occludes the active ingredients from the external environment, wherein the foam backing is the part of the patch that is placed on the skin, and wherein the release liner is the clear plastic sheet surrounding the patch that covers said foam backing, and wherein said release liner is removable by peeling it from the patch.

11. The dermal patch of claim 6, wherein the diclofenac is either diclofenac sodium or diclofenac epolamine.

12. The dermal patch of claim 6, wherein the diclofenac is either diclofenac sodium or diclofenac epolamine, and wherein the concentration of diclofenac sodium in the formulation is about 1.0 percent and wherein the concentration of diclofenac epolamine is about 1.3 percent.

13. The dermal patch of claim 6, wherein the concentration of capsaicin in the formulation is about 0.01%, about 0.025%, about 0.05%, about 0.01%, or about 0.015%.

14. The dermal patch of claim 6, wherein the total amount of eugenol is about 10 milligrams.

15. The dermal patch of claim 6, wherein the dermal patch is capable of reducing anxiety or depression that results from menstrual pain.

16. The dermal patch of claim 6, wherein the pharmaceutical formulation contains a total of about 35 milligrams of one or more cannabinoids or terpenes.

17. The dermal patch of claim 6, wherein the pharmaceutical formulation contains a total amount of about 10 milligrams of eugenol.

18. The dermal patch of claim 6, wherein the pharmaceutical formulation contains a total amount of about 35 milligrams of cannabinoids or terpenes.

19. The dermal patch of claim 16, wherein the terpene consists of β-caryophyllene (BCAP).

20. The dermal patch of claim 18, wherein the terpene consists of β-caryophyllene (BCAP).

21. The dermal patch of claim 6, wherein said pharmaceutical formulation excludes one or more or all of opioids, muscle relaxants, acetaminophen, corticosteroids, antianxiety drugs, antidepressants, and anticonvulsant drugs.

22. The dermal patch of claim 6, that includes one or more of a polyisobutylene (PIB) adhesive, ascorbyl palmitate, a tackifier in the form of a resin, and one or more permeation enhancers.

23. A pharmaceutical formulation that, when applied to the skin, is capable of reducing menstrual pain, wherein the composition comprises one of the following lists of chemical ingredients:
1) eugenol, capsaicin, and cannabidiol (CBD), but without CBDa,
2) eugenol, capsaicin, cannabidiol (CBD), and diclofenac, but without CBDa,
3) eugenol, capsaicin, cannabidiol (CBD), but without diclofenac and without CBDa,
4) eugenol, capsaicin, and diclofenac,
5) eugenol, capsaicin, without any diclofenac and without any cannabidiol (CBD),
6) eugenol, capsaicin, cannabidiol (CBD), cannabidiolic acid (CBDa),
7) eugenol, capsaicin, cannabidiol (CBD), cannabidiolic acid (CBDa), and diclofenac,
8) eugenol, capsaicin, cannabidiol (CBD), cannabidiolic acid (CBDa), but without diclofenac,
9) eugenol, capsaicin, without any diclofenac and without any cannabidiol (CBD),
10) eugenol, capsaicin, cannabidiolic acid (CBDa), but without CBD,
11) eugenol, capsaicin, cannabidiolic acid (CBDa), and diclofenac, but without CBD,
12) eugenol, capsaicin, cannabidiolic acid (CBDa), but without diclofenac, and without CBD,
13) eugenol, capsaicin, and diclofenac, but without CBD,
14) eugenol, capsaicin, and β-caryophyllene (BCAP),
15) eugenol, capsaicin, β-caryophyllene (BCAP), and diclofenac,
16) eugenol, capsaicin, β-caryophyllene (BCAP), but without diclofenac,
17) eugenol, capsaicin, without any diclofenac and without any β-caryophyllene (BCAP),
18) eugenol, capsaicin, β-caryophyllene (BCAP), cannabidiol (CBD),
19) eugenol, capsaicin, β-caryophyllene (BCAP), cannabidiolic acid (CBDa),
20) eugenol, capsaicin, β-caryophyllene (BCAP), cannabidiol (CBD), and diclofenac,
21) eugenol, capsaicin, β-caryophyllene (BCAP), cannabidiolic acid (CBDA), and diclofenac,
22) eugenol, capsaicin, β-caryophyllene (BCAP), cannabidiol (CBD), cannabidiolic acid (CBDA), and diclofenac,
23) eugenol, capsaicin, without any diclofenac and without any β-caryophyllene (BCAP),
24) eugenol, capsaicin, cannabidiolic acid (CBDa), but without β-caryophyllene (BCAP),
25) eugenol, capsaicin, cannabidiolic acid (CBDa), and diclofenac, but without β-caryophyllene (BCAP),
26) eugenol, capsaicin, cannabidiolic acid (CBDa), but without diclofenac, and without β-caryophyllene (BCAP),
27) eugenol, capsaicin, and diclofenac, but without β-caryophyllene (BCAP),
28) eugenol, capsaicin, without any diclofenac and without any β-caryophyllene (BCAP).
29) Any combination of one or more of eugenol, capsaicin, cannabidiol (CBD), cannabidiolic acid (CBDA), diclofenac, and β-caryophyllene (BCAP).

24. The pharmaceutical formulation of claim 23, wherein said capable of reducing menstrual pain is experienced by a patient who is treated with a dermal patch the delivers said pharmaceutical formulation, and wherein said reducing menstrual pain is as compared with said patient prior to attaching said dermal patch to said patient, and wherein the degree of menstrual pain prior to and after attaching said dermal patch to the skin of said patient is measured by a scoring tool.

25. The pharmaceutical formulation of 23, that excludes one or more or all of opioids, muscle relaxants, acetaminophen, corticosteroids, antianxiety drugs, antidepressants, and anticonvulsant drugs.

26. A dermal patch comprising one of the pharmaceutical formulations, as set forth in the following lists of chemical ingredients:
1) eugenol, capsaicin, and cannabidiol (CBD), but without CBDa,
2) eugenol, capsaicin, cannabidiol (CBD), and diclofenac, but without CBDa,
3) eugenol, capsaicin, cannabidiol (CBD), but without diclofenac and without CBDa,
4) eugenol, capsaicin, and diclofenac,
5) eugenol, capsaicin, without any diclofenac and without any cannabidiol (CBD),
6) eugenol, capsaicin, cannabidiol (CBD), cannabidiolic acid (CBDa),
7) eugenol, capsaicin, cannabidiol (CBD), cannabidiolic acid (CBDa), and diclofenac,
8) eugenol, capsaicin, cannabidiol (CBD), cannabidiolic acid (CBDa), but without diclofenac,
9) eugenol, capsaicin, without any diclofenac and without any cannabidiol (CBD),
10) eugenol, capsaicin, cannabidiolic acid (CBDa), but without CBD,
11) eugenol, capsaicin, cannabidiolic acid (CBDa), and diclofenac, but without CBD,
12) eugenol, capsaicin, cannabidiolic acid (CBDa), but without diclofenac, and without CBD,
13) eugenol, capsaicin, and diclofenac, but without CBD,
14) eugenol, capsaicin, and β-caryophyllene (BCAP),
15) eugenol, capsaicin, β-caryophyllene (BCAP), and diclofenac,
16) eugenol, capsaicin, β-caryophyllene (BCAP), but without diclofenac,
17) eugenol, capsaicin, without any diclofenac and without any β-caryophyllene (BCAP),
18) eugenol, capsaicin, β-caryophyllene (BCAP), cannabidiol (CBD),
19) eugenol, capsaicin, β-caryophyllene (BCAP), cannabidiolic acid (CBDa),
20) eugenol, capsaicin, β-caryophyllene (BCAP), cannabidiol (CBD), and diclofenac,
21) eugenol, capsaicin, β-caryophyllene (BCAP), cannabidiolic acid (CBDA), and diclofenac,
22) eugenol, capsaicin, β-caryophyllene (BCAP), cannabidiol (CBD), cannabidiolic acid (CBDA), and diclofenac,
23) eugenol, capsaicin, without any diclofenac and without any β-caryophyllene (BCAP),
24) eugenol, capsaicin, cannabidiolic acid (CBDa), but without β-caryophyllene (BCAP),
25) eugenol, capsaicin, cannabidiolic acid (CBDa), and diclofenac, but without β-caryophyllene (BCAP),
26) eugenol, capsaicin, cannabidiolic acid (CBDa), but without diclofenac, and without β-caryophyllene (BCAP),
27) eugenol, capsaicin, and diclofenac, but without β-caryophyllene (BCAP),
28) eugenol, capsaicin, without any diclofenac and without any β-caryophyllene (BCAP).
29) Any combination of one or more of eugenol, capsaicin, cannabidiol (CBD), cannabidiolic acid (CBDA), diclofenac, and B-caryophyllene (BCAP).
wherein said reducing menstrual pain is experienced by a patient treated with a dermal patch that delivers said pharmaceutical formulation, and wherein said reducing menstrual pain is with regard to the menstrual pain experienced by the same patient but prior to attaching said dermal patch to said patient, and wherein the degree of menstrual pain prior to and after attaching said dermal patch to the skin of said patient are both measured using the same scoring tool.

27. The dermal patch of claim 26, wherein said menstrual pain before and after treatment with said dermal patch is measured using the Visual Analogue Scale (VAS), Cox Menstrual Symptom Scale (CMSS)-interpreted menstruation symptoms score, Quality of Life Scale (QOLS)-interpreted quality of life score, the Flanagan Quality of Life (QOL) score, Verbal Multi-dimensional Scoring system (VMS) score, McGill Pain Questionnaire (MPQ) score, Profile of Mood States (POMS) Questionnaire, the Short Form-36 (SF-36) score, Profile of Mood States (POMS) Questionnaire score, or the Numeric Rating Scale (NRS) score.

28. The dermal patch of claim 26, that comprises a flexible tan polyethylene foam backing layer on a clear polyethylene terephthalate (PET) release liner, wherein the foam backing is the part of the patch that occludes the active ingredients from the external environment, wherein the foam backing is the part of the patch that is placed on the skin, and wherein the release liner is the clear plastic sheet surrounding the patch that covers said foam backing, and wherein said release liner is removable by peeling it from the patch.

29. The dermal patch of claim 26, wherein one or more or all of opioids, muscle relaxants, acetaminophen, corticosteroids, antianxiety drugs, antidepressants, and anticonvulsant drugs, are excluded from said pharmaceutical formulation.

30. The dermal patch of claim 26, that includes one or more of a polyisobutylene (PIB) adhesive, ascorbyl palmitate, a tackifier in the form of a resin, and one or more permeation enhancers.

31. The dermal patch of claim 30, wherein said one or more permeation enhancers comprises oleic acid, transcutol, isopentenyl pyrophosphate (IPP), dimethylsulfoxide (DMSO), propylene glycol (1,2 PG), or isopropyl myristate (IPM).

32. A method for manufacturing the dermal patch of claim 10, wherein the dermal patch is a monolithic-style patch, the method comprising:
Step (i). Mixing active pharmaceutical ingredients, wherein one or more of the active pharmaceutical ingredients are capable of reducing menstrual pain after transdermal administration, wherein said active pharmaceutical ingredients are dissolved in a solvent,
Step (ii). Mixing a pressure-sensitive adhesive dissolved in solvent with the above mixture of active pharmaceutical ingredients to create a homogenous mixture,
Step (ii). Degassing said mixture in a vacuum in order to remove residual air bubbles,
Step (iii). Using the solvent casting method, cast the mixture via knife-over-roll onto a silicone-treated polyethylene terephthalate (PET) release liner,
Step (iv). Evaporate the solvents in an oven, wherein said oven is kept about about 85 degrees centigrade for at least one minute, resulting in a dried coat, wherein said dried coat has a weight that is about 45 grams per square meter,
Step (v). Laminate the release liner to an occlusive backing, wherein the occlusive backing is made of polyethylene, polypropylene, or polyolefin foam,
Step (vi) Cut the assembled release liner and occlusive backing into rectangles that have rounded corners, wherein the rectangles have an area of about 50 square centimeters.

33. The method of claim 32, wherein said active pharmaceutical ingredients comprises any combination of one or more of eugenol, capsaicin, cannabidiol (CBD), cannabidiolic acid (CBDA), diclofenac, and β-caryophyllene (BCAP).

34. The method of claim 32, wherein the mixing step includes adding one or more permeation enhancers.

35. The method of claim 32, wherein said mixing comprising using an ultra-high shear dispersion blade.

36. The method of claim 32, wherein the solvent for dissolving said active pharmaceutical ingredients comprises ethanol or methanol.

37. The method of claim 32, wherein said adhesive is a tackifier.

38. The method of claim 32, wherein the mixing step includes adding one or more preservatives, wherein said one or more preservatives are optionally dissolved in ethanol, methanol, or heptane.

39. The method of claim 32, wherein the mixing step includes adding a tackifier adhesive dissolved in heptane.

40. A method for manufacturing the dermal patch of claim 26, wherein the dermal patch is a monolithic-style patch, the method comprising:
Step (i). Mixing active pharmaceutical ingredients, wherein one or more of the active pharmaceutical ingredients are capable of reducing menstrual pain after transdermal administration, wherein said active pharmaceutical ingredients are dissolved in a solvent,
Step (ii). Mixing a pressure-sensitive adhesive dissolved in solvent with the above mixture of active pharmaceutical ingredients to create a homogenous mixture,
Step (ii). Degassing said mixture in a vacuum in order to remove residual air bubbles,
Step (iii). Using the solvent casting method, cast the mixture via knife-over-roll onto a silicone-treated polyethylene terephthalate (PET) release liner,
Step (iv). Evaporate the solvents in an oven, wherein said oven is kept about about 85 degrees centigrade for at least one minute, resulting in a dried coat, wherein said dried coat has a weight that is about 45 grams per square meter,
Step (v). Laminate the release liner to an occlusive backing, wherein the occlusive backing is made of polyethylene, polypropylene, or polyolefin foam,
Step (vi) Cut the assembled release liner and occlusive backing into rectangles that have rounded corners, wherein the rectangles have an area of about 50 square centimeters.

41. The method of claim 40, wherein said active pharmaceutical ingredients comprises any combination of one or more of eugenol, capsaicin, cannabidiol (CBD), cannabidiolic acid (CBDA), diclofenac, and β-caryophyllene (BCAP).

42. The method of claim 40, wherein the mixing step includes adding one or more permeation enhancers.

43. The method of claim 40, wherein said mixing comprising using an ultra-high shear dispersion blade.

44. The method of claim 40, wherein the solvent for dissolving said active pharmaceutical ingredients comprises ethanol.

45. The method of claim 40, wherein said adhesive is a tackifier.

46. The method of claim 40, wherein the mixing step includes adding one or more preservatives, wherein said one or more preservatives are optionally dissolved in ethanol.

47. The method of claim 6, wherein the mixing step includes adding a tackifier adhesive dissolved in hexane.

48. A method for manufacturing the dermal patch of claim 6, wherein the dermal patch is a reservoir-style patch, wherein said reservoir contains a formulation, wherein said formulation includes one or more anti-pain drugs, the method comprising the steps of
Step (i) Making the reservoir by feeding two strips (first strip and second strip) into a machine with rollers, wherein the rollers move the strips at the same speed, wherein a first face of the first strip is caused to contact a first face of the second strip, in preparation for heating the edges of the two strips thus sealing them together,
Step (ii) The step of using heaters resembling wheels or rollers to clamp down on the edges of the two strips, thereby creating a sandwich taking the form of a long closed tube,
Step (iii) The step of using heaters resembling wheels or rollers to clamp down on the edges of the two strips, thereby creating a sandwich taking the form of a long closed tube,
Step (iv) The step of using transverse clamps to create separate pouches in the long closed tube, wherein the transverse clamps are heated and clamp down, thereby creating an unfilled pouch, Docket No. RBI-010500-PRO 84
Step (v) The step of filling the unfilled pouch using a long tube that reaches down into the long closed tube, wherein said long tube fills each pouch as it is created, wherein said machine has a deposit station, and wherein said step of filling is performed at the deposit station,
Step (vi) The step of using said heated transverse clamps to clamp down on a previously-filled pouch, thereby creating a top seal on said previously-filled pouch, thereby creating a filled reservoir,
Step (vii) The step of providing a backing, a permeable layer, and a filled reservoir, wherein the backing has edges and the permeable layer has edges,
Step (viii) The step of contacting the filled reservoir to the backing and to the permeable layer, wherein said contacting further comprises attaching by way of an adhesive seal or by way of a heat seal, wherein permeable layer resides on the proximal side (side closer to the skin) of the reservoir, and wherein the backing resides on the distal side (side farther from the skin) of the reservoir,
Step (ix) The step of attaching the edges of the backing to the edges of the permeable layer to each other to prevent leaking of said formulation out of the dermal patch,

49. The method of claim 48, wherein said one more anti-pain drugs include one or more of eugenol, capsaicin, cannabidiol (CBD), cannabidiolic acid (CBDA), diclofenac, and β-caryophyllene (BCAP).

50. The method of claim 48, wherein said formulation comprises a penetration enhancer.

51. The method of claim 48, wherein said formulation comprises a preservative.

52. The method of claim 48, wherein said formulation comprises an antioxidant such as ascorbyl palmitate, butylated hydroxytoluene (BHT), butylated hydroxyanisole (BHA), Tocopherol, ascorbyl palmitate, propyl gallate, ascorbic acid, or citric acid.

53. The method of claim 48, wherein the permeable layer comprises permeable polypropylene film, permeable polyolefin film, permeable polyethylene film, or permeable urethane film.

54. A method for manufacturing the dermal patch of claim 26, wherein the dermal patch is a reservoir-style patch, wherein said reservoir contains a formulation, wherein said formulation includes one or more anti-pain drugs, the method comprising the steps of:
Step (i) Making the reservoir by feeding two strips (first strip and second strip) into a machine with rollers, wherein the rollers move the strips at the same speed, wherein a first face of the first strip is caused to contact a first face of the second strip, in preparation for heating the edges of the two strips thus sealing them together,
Step (ii) The step of using heaters resembling wheels or rollers to clamp down on the edges of the two strips, thereby creating a sandwich taking the form of a long closed tube,
Step (iii) The step of using heaters resembling wheels or rollers to clamp down on the edges of the two strips, thereby creating a sandwich taking the form of a long closed tube,
Step (iv) The step of using transverse clamps to create separate pouches in the long closed tube, wherein the transverse clamps are heated and clamp down, thereby creating an unfilled pouch,
Step (v) The step of filling the unfilled pouch using a long tube that reaches down into the long closed tube, wherein said long tube fills each pouch as it is created, wherein said machine has a deposit station, and wherein said step of filling is performed at the deposit station,
Step (vi) The step of using said heated transverse clamps to clamp down on a previously-filled pouch, thereby creating a top seal on said previously-filled pouch, thereby creating a filled reservoir,
Step (vii) The step of providing a backing, a permeable layer, and a filled reservoir, wherein the backing has edges and the permeable layer has edges,
Step (viii) The step of contacting the filled reservoir to the backing and to the permeable layer, wherein said contacting further comprises attaching by way of an adhesive seal or by way of a heat seal, wherein permeable layer resides on the proximal side (side closer to the skin) of the reservoir, and wherein the backing resides on the distal side (side farther from the skin) of the reservoir,
Step (ix) The step of attaching the edges of the backing to the edges of the permeable layer to each other to prevent leaking of said formulation out of the dermal patch.

55. The method of claim 54, wherein said one more anti-pain drugs include one or more of eugenol, capsaicin, cannabidiol (CBD), cannabidiolic acid (CBDA), diclofenac, and β-caryophyllene (BCAP).

56. The method of claim 54, wherein said formulation comprises a penetration enhancer.

57. The method of claim 54, wherein said formulation comprises a preservative.

58. The method of claim 54, wherein said formulation comprises antioxidant such as ascorbyl palmitate, butylated hydroxytoluene (BHT), butylated hydroxyanisole (BHA), Tocopherol, ascorbyl palmitate, propyl gallate, ascorbic acid, or citric acid.

59. The method of claim 54, wherein the permeable layer comprises permeable polypropylene film, permeable polyolefin film, permeable polyethylene film, or permeable urethane film.
